(19) United States
(12) Patent Application Publication
(10) Pub. No.: US 2005/0191747 A1
(43) Pub. Date: Sep. 1, 2005

(54) Recombinant bovine immunodeficiency virus based gene transfer system

(76) Inventors: Tianci Luo, Clarksville, MD (US); Michael Kaleko, Rockville, MD (US); Douglas Golightly, Frederick, MD (US); Rene Molina, Frederick, MD (US); Mengtao Li, Lexington, KY (US); George Lambrou, Basel (CH)

Correspondence Address: BELL, BOYD, & LLOYD LLC, P. O. BOX 1135, CHICAGO, IL, 60690-1135, US

(21) Appl. No.: 10/910,293

(22) Filed: Aug. 4, 2004

Related U.S. Application Data
(63) Continuation of application No. PCT/US03/03307, filed on Feb. 4, 2003.
(60) Provisional application No. 60/353,177, filed on Feb. 4, 2002.
(60) Provisional application No. 60/433,956, filed on Dec. 18, 2002.

(51) Int. Cl. C12N 15/867; C12N 7/00

(52) U.S. Cl. 435/456; 435/325; 435/320.1; 435/235.1

(57) ABSTRACT

The present invention provides recombinant lentiviral vectors and gene transfer systems which produce said vectors, cell lines utilized in the production of said recombinant lentiviral vectors, and Bovine Immunodeficiency Virus DNA sequences utilized in the recombinant vectors and gene transfer systems.

This application claims the benefit of U.S. Provisional Application No.60/353,177, filed Feb. 4,2002, and U.S. Provisional Application No. 60/433,956, filed Dec. 18, 2002, both of which are incorporated herein by reference in their entireties.

### FIELD OF THE INVENTION

The present invention relates generally to the field of viral vectors and more specifically to novel recombinant lentiviral vectors, gene transfer systems which produce the vectors and cell lines for expression of the gene transfer systems and packaging and delivery of the recombinant vectors.

### BACKGROUND OF THE INVENTION

The publications and other materials used herein to illuminate the background of the invention, and in particular, cases to provide additional details respecting the practice of the invention are incorporated herein by reference, and for convenience, are referenced by author and date in the following text and respectively grouped in the appended List of References.

Lentiviruses contain the genes gag, pol, env and other genes with regulatory or structural function. Lentiviruses can infect both dividing and non-dividing cells, in contrast to oncoretroviruses, for example, which can only infect dividing cells. It is the ability to infect non-dividing cells which makes lentiviruses an especially useful system for in vivo and ex vivo gene therapy.

An important consideration in using lentiviruses for gene therapy is the availability of “safe” lentiviruses for use as vectors. Safe retroviral vectors are capable of delivering a gene of interest to a cell but have a reduced ability to generate replication competent viral particles during this process. One of the ways these vectors are made safe is to isolate essential viral genes on separate DNA constructs, wherein essential genes required for packaging of viral RNA, such as the gag, pol and env genes, are provided on different DNA constructs from the DNA sequences and genes required for delivery of the heterologous gene of interest to an infected cell nucleus and chromosomes. Packaging cell lines and vector producing cell lines have been developed to meet this need. Briefly, this methodology employs the use of two components, a lentiviral vector and a packaging cell line. The lentiviral vector contains long terminal repeats (LTRs) which are necessary for integration of the proviral DNA into a host chromosome, the heterologous nucleotide sequence to be transferred and a packaging sequence which enables packaging of the viral RNA into infectious but replication deficient vectors. Viral vectors utilized in gene delivery to eukaryotic cells generally are constructed so that many essential viral genes are deleted and replaced by a gene of interest. A replication deficient lentiviral vector will not reproduce by itself because the genes which encode structural and envelope proteins such as GAG, POL and ENV are not included within the vector genome. The genes which have been deleted from the vector are generally provided by one or more helper or packaging constructs in a packaging cell line. The packaging cell line contains genes encoding the essential GAG, POL, and ENV proteins, but these gene constructs do not contain a packaging signal (also referred to herein as a “packaging sequence”). Thus, a packaging cell line can only form empty virion particles by itself. In order to package a gene of interest for delivery to a target cell, however, the essential viral genes must be provided in trans so that the recombinant viral vector construct can be assembled into an infectious yet replication defective vector.

When a lentiviral vector construct having a packaging signal is introduced into a packaging cell line, the cell line will produce vector particles containing the lentiviral vector construct's genome, without the other essential lentiviral genes. By removing essential genes from the vector construct, the infectious virus particles or vectors produced are capable of delivering the heterologous gene of interest to infected cells without generating replication competent viruses. In this manner, only when the essential genes are provided in trans will a host cell produce recombinant replication deficient vectors containing the vector construct having the gene of interest (PCT Application No. PCT/US00/33725 (WO 01/44458)).

There are, however, several shortcomings with the current use of vector and packaging construct cell lines. One issue involves the generation of replication competent lentiviruses by the producer cells. Briefly, replication competent viruses can be produced in conventional producer cells when, for example, the construct containing the vector DNA and the construct containing the other essential viral genes recombine with each other, or when the vector DNA or the construct containing the other essential viral genes recombines with homologous cryptic endogenous retroviral elements in the producer cell. Furthermore, if a recombinant lentiviral vector encounters homologous sequences in a host cell following transfection with the vector constructs, the infected host cells could possibly allow recombination among the vector genome and endogenous viral sequences present in the host cell.

One recent approach to constructing safer packaging cell lines involves the use of complementary portions of helper virus elements, divided among two separate plasmids, one containing gag and pol, and the other containing env (see, Markowitz et al., J. Virol. 62:1120-1124; and Markowitz et al., Virology 167:600-606, 1988). One benefit of this double-plasmid system is that three recombination events are required to generate a replication competent genome. This approach minimizes the ability for co-packaging and subsequent transfer of the multiple components of the wildtype viral genome, as well as significantly decreasing the frequency of recombination due to the presence of distinct DNA components which comprise the recombinant lentiviral system in the packaging cell. Nevertheless, the double-component system suffers from the drawback of including portions of DNA homologous to the vector construct and thus retains the possibility of producing replication competent virus via homologous recombination between the constructs.

Gene transfer systems based on Human Immunodeficiency Virus (HIV) are by far the most developed lentivirus systems, with documented in vivo transduction of rat brain, retina and muscle and liver cells. However, HIV is the causative agent of AIDS. In addition, HIV-1 derived vectors have transduced human corneal tissue ex vivo and unstimulated hematopoietic stem cells transfected in vitro have developed into mature T and B cells in in vivo models of lymphocyte development (Douglas, et al., Hum Gene Ther. 12(4):401-413 (2001); Miyoshi, et al., Virol. 72:8150-8157 (1999)). HIV-based recombinant lentiviral vectors pose various safety concerns for gene therapy. For example, it has been postulated that if a replication defective HIV vector were to recombine with endogenous human lentivirus latently or transiently infecting a cell, there would be a chance of generating replication competent HIV. The chances of a non-human or especially a non-primate lentivirus such as BIV encountering homologous viral sequences in the same host cell is far less likely to occur.

To circumvent the safety concern associated with HIV-based lentiviral vectors, animal lentiviruses such as feline immunodeficiency virus (FIV), equine infectious anemia virus (EIAV), visna virus, have been used for generation of gene transfer vectors. However, it has been shown that the vectors derived from these animal lentiviruses do not perform as well as vectors derived from HIV (Price M A et al., 2002, Molecular Therapy; O'Rourke J P et al., 2002, Journal of Virology; Ikeda Y et al., 2002, Gene Therapy; Berkowitz R D et al., 2001, Virology).

Bovine immunodeficiency virus (BIV) is classified in the retroviral subfamily lentiviridae. Lentiviruses are exogenous non-oncogenic retroviruses and include inter alia, Equine Infectious Anemia Virus (EIAV), Simian Immunodeficiency Virus (SIV) visna and progressive pneumonia viruses of sheep, feline immunodeficiency virus (FIV) and human immnunodeficiency viruses (HIV-1 and HIV-2). Among the lentiviruses, there is a distinct order of homology between the families.

Bovine immunodeficiency virus shares no significant overall homology with HIV, SIV, FIV, EIAV, Visna virus (Garvey K J et al., 1990, Virology; Gonda M A et al., 1994, Virus Research). BIV is the most distant lentivirus from HIV and SIV in a phylogenetic analysis (Gonda M A et al., 1994, Virus Research). Unlike other lentiviruses, BIV has not been extensively studied, locations of some important BIV elements such as the packaging signal sequence and Rev response element (RRE) have not been identified. Therefore, it presents a significant challenge to derive an advanced gene transfer system from this virus. After extensive research, the BIV packaging signal sequence and RRE sequence were mapped in this invention and advanced BIV vectors were generated. The vectors that were derived from BIV performed as well as HIV-based vectors in titer, transduction efficiencies and duration of gene expression in vitro and in vivo.

BIV infects cows and the deleterious effects are unclear. Like HIV, BIV has accessory genes but most are distinct from those of HIV. BIV is phylogenetically distinct from HIV and does not readily infect T cells. It is predominantly found in monocytes and splenic macrophages in vivo. The G-protein of vesicular-stomatitis virus (VSV-G) efficiently forms pseudotyped virions with genome and matrix components of other viruses. Recombinant BIV viral constructs containing the envelope of VSV-G have successfully been introduced into human cells with efficiencies of transduction and expression of heterologous genes that approach those seen with HIV gene transfer systems (Berkowitz et al. J. Virol. 7(7):3371-3382 (2001)).

U.S. Pat. No. 6,277,633, issued to Olsen, describes a recombinant lentiviral vector expression system based on EIAV comprising a first vector that is a gag/pol expression vector, a second vector having cis-acting sequence elements required for reverse transcription of the vector genome, a packaging sequence, and additionally containing a multiple cloning site wherein a heterologous gene can be inserted. The system described in Olsen also utilizes a third vector which expresses a viral envelope protein. The first and third vectors are packaging signal-defective.

The present invention provides an optimized gene transfer system based on the BIV genome for transfer of heterologous genes to a wide range of eukaryotic cells. The system, vectors and packaging cell lines of the invention have been designed to minimize homology among various constructs of the system, thereby reducing the likelihood of generating a replication competent BIV vector.

### SUMMARY OF THE INVENTION

The invention comprises gene transfer systems in which BIV lentiviral packaging genes and cis-acting genes necessary for packaging of viral RNA and integration of proviral DNA into infected cell chromosomes are provided on distinct DNA constructs, wherein one or more constructs contain one or more BIV packaging genes which can complement additional DNA constructs to provide replication-defective infectious particles for delivering heterologous genes of interest to a target cell. The various components of the constructs can be provided on the same or different DNA molecules and can comprise three, four or five separate constructs.

In one embodiment, the invention comprises a recombinant lentiviral gene transfer system wherein the genes necessary for production of recombinant replication deficient vectors are provided on three distinct DNA constructs, the system comprising: a packaging construct comprising a BIV gag gene and a BIV pol gene; a viral surface protein gene construct comprising a viral surface protein gene; and a transfer vector construct comprising a DNA segment comprising a heterologous gene of interest and a minimal BIV packaging sequence for packaging of the heterologous gene of interest into replication deficient vectors. In a preferred embodiment, the invention provides a three construct system comprising:
(a) a packaging construct comprising a DNA segment comprising a first promoter operably linked to a BIV gag gene and a BIV pol gene;
(b) a viral surface protein gene construct comprising a DNA segment comprising a second promoter operably linked to a viral surface protein gene; and
(c) a transfer vector construct comprising a DNA segment comprising a third promoter operably linked sequentially to a first R region, a U5 region, a UTR (Untranslated region) region, a minimal BIV packaging sequence, an RRE sequence, a fourth promoter operably linked to a heterologous gene of interest, a 3′ polypurine tract region, a U3 region, a second R region and optionally a second U5 region;
a rev gene located on one of the packaging, viral surface protein gene, and transfer vector constructs; wherein all of the promoters may be the same or different.

In another embodiment, the invention comprises a recombinant lentiviral gene transfer system wherein the genes necessary for production of recombinant replication deficient vectors are provided on four distinct DNA expression constructs, the system comprising a packaging construct comprising a DNA segment comprising a BIV gag gene and a BIV pol gene; a rev construct comprising a DNA segment comprising a BIV rev gene; a viral surface protein gene construct comprising a DNA segment comprising a viral surface protein gene; and a transfer vector construct comprising a DNA segment comprising a heterologous gene of interest and a BIV packaging sequence for packaging of the heterologous gene of interest into replication deficient vectors.

In another preferred embodiment, the invention provides a four construct system comprising: recombinant lentiviral gene transfer system comprising:
(a) a packaging construct comprising a DNA segment comprising a first promoter operably linked to a BIV gag gene and a BIV pol gene;
(b) a viral surface protein gene construct comprising a DNA segment comprising a second promoter operably linked to a viral surface protein gene;
(c) a rev construct comprising DNA segment comprising a third promoter operably linked to a rev gene; and
(d) a transfer vector construct comprising a DNA segment comprising: a fourth promoter operably linked sequentially to a first R region, a U5 region, a UTR region, a minimal BIV packaging sequence, an RRE sequence, a fifth promoter operably linked to a heterologous gene of interest, a 3′ polypurine tract region, a U3 region, a second R region and optionally a second U5 region; wherein all of the promoters may be the same or different.

In yet a further embodiment, the invention comprises a recombinant lentiviral gene transfer system wherein the genes necessary for production of recombinant replication deficient vectors are provided on five distinct DNA expression constructs, the system comprising a first packaging construct comprising a BIV gag gene; a second packaging construct comprising a BIV pol gene; a rev construct comprising a rev gene; a viral surface protein gene construct comprising a viral surface protein gene; and a transfer vector construct comprising a DNA segment comprising a heterologous gene of interest and a BIV packaging sequence for packaging of the heterologous gene of interest into replication deficient vectors.

In another preferred embodiment, the invention provides a five construct system comprising:
(a) a first packaging construct comprising a DNA segment comprising a first promoter operably linked to a DNA segment comprising a BIV gag gene;
(b) a second packaging construct comprising a DNA segment comprising a second promoter operably linked to a DNA segment comprising a BIV pol gene;
(c) a viral surface protein gene construct comprising a DNA segment comprising a third promoter operably linked to a viral surface protein gene;
(d) a rev construct comprising a fourth promoter operably linked to a rev gene; and
(e) a transfer vector construct comprising a DNA segment comprising a fifth promoter operably linked sequentially to a first R region, a US region, a UTR region, a BIV packaging sequence, an RRE sequence, a sixth promoter operably linked to a heterologous gene of interest, a 3′ polypurine tract region, a U3 region, a second R region and optionally a second U5;
wherein all of the promoters may be the same or different.

In one embodiment, one or more of the promoters is a regulatable promoter. For example, in several exemplary embodiments, the regulatable promoter is selected from the group consisting of inducible and repressible, inducible, repressible and tissue-specific promoters. In another embodiment, the promoter operably linked to the heterologous gene is a constitutive promoter. In another embodiment, the promoter operably linked to the heterologous gene is a regulatable promoter. In another embodiment, the promoter operably linked to the heterologous gene is a tissue-specific promoter. In another embodiment, the promoter operably linked to the heterologous gene is inducible or repressible. In another embodiment, the promoter operably linked to at least one of the gag or pol genes are regulatable. In another embodiment, the promoters operably linked to at least one of the gag or pol genes are inducible or repressible. In another embodiment, the promoter operably linked to the viral surface protein gene is regulatable. In another embodiment, the promoter operably linked to the viral surface protein gene is inducible or repressible.

In a preferred embodiment, the transfer vector construct having a BIV packaging sequence and a heterologous gene of interest comprises a promoter operably linked to a first R region, a U5 region, a UTR region, a BIV packaging sequence, a BIV RRE, a promoter operably linked to the heterologous gene of interest, a U3 region and a second R region and, optionally, a second U5 region.

In one embodiment, the various gene transfer systems of the invention comprise recoded (codon optimized) nucleic acids sequences, wherein the recoded sequences encode wildtype BIV gene functions, yet have reduced sequence homology between the different DNA constructs of the system when compared to previous BIV gene transfer systems.

In yet another embodiment, the various gene transfer systems of the invention comprise recoded nucleic acid sequences, wherein the recoded sequences encode wildtype BIV gene functions while comprising recoded codon usage optimal for translation of protein in eukaryotic cells. In a particularly preferred embodiment, the eukaryotic cells are human cells.

The invention further provides a packaging cell and packaging cell lines utilizing the gene transfer systems of the present invention. The packaging cell and cell lines comprise the packaging construct or constructs and the viral surface protein gene construct.

The invention further provides a producer cell and producer cell lines utilizing the gene transfer systems of the present invention. The producer cell and cell lines comprise the packaging construct or constructs, the viral surface protein gene construct and the transfer vector construct.

The invention additionally provides methods for producing replication deficient recombinant lentiviral vectors utilizing the gene transfer systems, cells and cell lines of the present invention.

The invention also provides vectors generated by expression of the gene transfer systems of the invention in producer cells. The addition of a transfer vector construct to a packaging cell results in a producer cell. The producer cell produces virions or vectors that contain the vector RNA. Infection of a cell with the vector results in the transfer of the heterologous gene of interest to the cell and expression of the heterologous gene of interest in the cell.

In yet another embodiment, the invention provides methods for treating an animal by contacting cells of the animal with replication deficient recombinant lentiviral vectors of the invention. Contact of animal cells can occur in vivo or in vitro.

The invention further comprises recombinant lentiviral gene transfer systems with improved safety for use in humans wherein the BIV based vector RNA of the system cannot be packaged into infectious vectors when introduced into cells encoding and expressing the packaging genes of HIV such as by infection with wild-type HIV.

The invention further provides gene transfer vectors and methods of gene transfer and expression which can be used to alter the expression patterns of genes in the study of gene function in particular cell types.

In accordance with this invention, the genetic location on the BIV genome which contains the minimal nucleic acid sequence of the BIV packaging sequence necessary for efficient packaging of viral RNA into infectious vectors has been determined.

The genetic location on the BIV genome which contains the cPPT (Central Polypurine Tract) that facilitates entry of lentiviral preintegration complex containing proviral nucleic acid into the nuclear membrane of infected cells has also been determined. It has been shown that cPPT enhances nuclear import of lentiviral preintegration complex into non-dividing cells although cPPT is not absolutely necessary for a lentiviral vector to transduce non-dividing cells. The genetic location on the BIV genome which contains the ribosomal frame shifting site which enables cotranslation of the BIV gag and pol genes from a single mRNA transcript also has been determined.

In one embodiment, knowledge of the frame shift site between the gag and pol genes has been used to recode these genes. As a result, the recoded gag and pol are translated with greater efficiency in host cells, providing a recombinant lentiviral gene transfer system with increased viral titer. Also, the recoded gag and pol genes provide a recombinant lentiviral gene transfer system with a reduction in homology between the packaging construct and the vector construct. Furthermore, without being bound by theory, the recoded gag and pol gene sequence is believed to have an altered secondary structure in the mRNA expressed therefrom, thereby providing a recombinant lentiviral gene transfer system that does not require an RRE sequence to functionally express gag and pol in vector producing cells. Elimination of the RRE from the gag/pol constructs eliminates all homology with the RRE sequence in the vector construct, thereby further diminishing the likelihood of generating a replication competent recombinant lentivirus. This aspect of the invention provides an additional measure of safety.

The RNA coding sequence for the pol gene of BIV also has been determined. In one embodiment, the invention provides the nucleic acid sequence consisting of wild-type pol as shown in SEQ ID NO:50. In another embodiment, the invention provides a recoded pol sequence as shown in SEQ ID NO: 52. In another embodiment, the invention provides an isolated nucleic acid encoding the polypeptide sequence of the BIV pol gene product as shown in SEQ ID NO:51. In yet another embodiment, the invention provides the synthetic nucleic acid shown in SEQ ID NO: 53, which is a synthetic sequence which adds an ATG codon to the wildtype BIV pol gene sequence. In yet another embodiment, the invention provides the synthetic nucleic acid shown in SEQ ID NO: 54, which is a synthetic sequence which adds an ATG codon to the recoded BIV pol gene sequence.

In another embodiment the gag/pol constructs of the invention contain a mutation as described in PCT Application PCT/EP02/02807 (WO 02/072851) wherein the protease encoding sequence includes a mutation corresponding to a T26S substitution in the encoded lentiviral protease.

The genetic location on the BIV genome for the rev gene has also been determined. Utilizing knowledge of the location of rev, DNA was synthesized encoding the rev gene in a single open reading frame. Accordingly, recombinant lentiviral gene transfer systems have been constructed in which the rev gene is provided on a separate expression construct from gag and pol. The rev gene can be expressed as a spliced message contained in two exons or alternatively as a single message encoded in one exon and open reading frame.

The genetic location on the BIV genome for the BIV RRE also has been determined. A 312 nucleotide sequence located in the BIV env region contains the BIV RRE gene sequence, as shown in SEQ ID NO:40.

The invention further comprises culturing producer cells in a culture medium comprising inhibitors of histone deacetylase in the cell culture medium, thereby generating recombinant replication deficient vectors with a higher titer and greater infectivity of host cells when compared to viral vectors cultured without histone deacetylase inhibitors.

### BRIEF DESCRIPTION OF THE FIGURES

FIG. 1 is a schematic representation of the BIV three construct gene transfer system.

FIG. 2 is a schematic representation of the BIV four construct gene transfer system.

FIG. 3 shows flow cytometry analysis of eGFP expression in cells transduced with BIV vectors containing different amounts of the gag sequence. Panel A) Mock infected, B) BIV vector from pBSV4MGppt, C) BIV vector from pBIVminivec, D) BIV vector from pBV28 containing 28 bps of gag sequence, E) BIV vector from pBV54 containing 54 bps of gag sequence,F) BIV vector from pBV101 containing 101 bps of gag sequence.

FIG. 4 shows a functional comparison of transduction efficiency by BIV vector containing either BIV or HIV cPPT.

FIG. 5 shows the BIV Pol translational ribosomal frameshifting site.

FIG. 6 shows a schematic representation of the recoded BIV gag/pol expression construct.

FIG. 7 shows the results of adding a histone deacetylase inhibitor during viral vector production on the production of Bovine Immunodeficiency Virus based lentiviral vectors.

FIG. 8 shows the results of adding a histone deacetylase inhibitor during viral vector production on the transduction efficiency of Bovine Immunodeficiency Virus based lentiviral vectors.

### BRIEF DESCRIPTION OF THE SEQUENCE LISTING

The Sequence Listing associated with the instant disclosure is hereby incorporated by reference into the instant disclosure. The following is a description of the sequences contained in the Sequence Listing:

SEQ ID NO:1 Bovine Immunodeficiency Virus.

SEQ ID NO:2-9 Oligonucleotides.

SEQ ID NO:10 Rev gene.

SEQ ID NO:11-38 Oligonucleotides.

SEQ ID NO:39 BIV packaging signal.

SEQ ID NO:40 312 bp of BIV env sequence contains BIV RRE sequence.

SEQ ID NO:41-48 Oligonucleotides.

SEQ ID NO:49 DNA sequence of recoded BIV gag/pol.

SEQ ID NO:50 BIV pol DNA sequence.

SEQ ID NO:51 BIV pol amino acid sequence.

SEQ ID NO:52 Recoded BIV pol DNA sequence.

SEQ ID NO:53 Wild type BIV Pol sequence with ATG.

SEQ ID NO:54 Recoded BIV pol DNA sequence with ATG.

SEQ ID NO:55 Partial amino acid sequence of HIV protease.

SEQ ID NO:56 Partial amino acid sequence of BIV protease.

SEQ ID NO:57 Partial amino acid sequence of mutated HIV HXB2.protease.

SEQ ID NO:58 Partial amino acid sequence of mutated BIV protease.

SEQ ID NO:59 Recoded gag/pol with protease mutation.

SEQ ID NO:60 Mouse RdCVF1 cDNA.

SEQ ID NO:61 Amino acid sequence of translated mouse RdCVF1 cDNA.

SEQ ID NO:62 Human RdCVF1 cDNA.

SEQ ID NO:63 Amino acid sequence of translated human RdCVF1 cDNA.

SEQ ID NO:64 Mouse RdCVF2 cDNA.

SEQ ID NO:65 Amino acid sequence of translated mouse RdCVF2 cDNA.

SEQ ID NO:66 Human RdCVF2 cDNA.

SEQ ID NO:67 Amino acid sequence of translated human RdCVF2 cDNA.

SEQ ID NO:68 Thogoto virus envelope.

SEQ ID NO:69 Amino acid sequence of translated Thogoto virus envelope.

SEQ ID NO:70 Recoded Thogoto virus envelope.

SEQ ID NO:71 Amino acid sequence of translated recoded Thogoto virus envelope.

### DETAILED DESCRIPTION OF THE INVENTION

The practice of the invention will employ, unless otherwise indicated, conventional techniques of cell biology, molecular biology, cell culture, virology, immunology and the like which are well known by those skilled in the art. These techniques are fully disclosed in current literature and reference is made, for example, to Molecular Cloning, A Laboratory Manual, 2nd Ed., Sambrook et al. (1989); Cell Biology, A Laboratory Handbook, Celis (1994); Bahnson et al., J. of Virol. Methods, 54:131-143 (1995); Culture of Animal Cells, A Manual of Basic Techniques. Freshney (1994); Rigg et al., Virology 218:290-295.

As used herein, the singular form “a”, “an”, and “the” include plural references unless the context clearly indicates otherwise. For example, a reference to “a vector particle” would include a plurality of vector particles.

The term “construct” refers to a DNA sequence usually in the context of a plasmid, but multiple constructs can be provided on the same plasmid.

The term “gene” and “coding sequence” are used interchangeably herein and refer to an “open reading frame” that encodes a protein.

The term “defective” as used herein refers to a viral vector or nucleic acid sequence that is not functional or has a decreased functionality in comparison to wildtype with regard to biological activity, encoding or expressing its gene products or serving as a cis-acting nucleic acid sequence. To illustrate with non-limiting examples: a defective env gene sequence will not encode the ENV protein; a defective packaging signal will not facilitate the packaging of the nucleic acid molecule the defective signal is located on at the same efficiency as the native packaging signal; and a replication “defective” lentiviral particle will not be capable of replicating and producing new infectious viral particles following entry into a host cell. Nucleic acid sequences can be made defective by any means known in the art, including by the deletion of some or all of the sequence, by placing the sequence out-of-frame, or by otherwise blocking the sequence.

As used herein, the terms “deleted” or “deletion” mean either total deletion of the specified segment or the deletion of a sufficient portion of the specified segment to render the segment inoperative or nonfunctional, in accordance with standard usage. The term “replication defective” as used herein, means that the constructs that encode BIV structural proteins cannot be encapsidated or are encapsidated at negligible levels in the producer or packaging cell. The resulting lentivirus particles are replication defective inasmuch as the packaged vector does not include all of the viral structural proteins required for encapsidation, at least one of the required structural proteins being deleted therefrom, such that the packaged vector is not capable of replicating the entire viral genome.

The phrases “essential genes” or “BIV essential genes” as used herein refer to the genes which encode the proteins which are required for encapsidation (e.g., packaging) of the BIV genome to generate infectious lentiviral particles, and include gag, pol, env and rev, cis-acting elements which are required for reverse transcription of vector genomic RNA into proviral DNA and integration of the proviral DNA into a target cell genome (e.g. BIV LTR).

An “expression construct” refers to a DNA segment that comprises one or more genes or portions of a gene that are contained on the DNA segment wherein the gene or portions of genes can include a combination of promoter and enhancer regions, including all accessory regions for transcription and translation of an encoded protein or nucleic acid as known in the art, an open reading frame encoding a protein, a cis acting regulatory element and the like. In addition, such constructs can contain an origin of replication so that the entire construct can replicate in a host cell. The constructs of the present invention are provided on one or more DNA vectors. In a preferred embodiment, each construct of the present invention is provided on separate DNA molecules.

A “viral surface protein gene construct” refers to a DNA segment that encodes and expresses a viral surface protein gene.

The term “nucleic acid sequence” or “gene sequence,” as used herein, is intended to refer to a nucleic acid molecule (preferably DNA or RNA). Such nucleotide sequences can be derived from a variety of sources including genomic DNA, cDNA, synthetic DNA, proviral DNA, viral RNA, mRNA, synthetic RNA or combinations thereof. Such gene sequences can comprise genomic DNA which may or may not include naturally occurring introns. Moreover, such genomic DNA can be obtained in association with promoter sequences or poly-adenylation sequences. Genomic or cDNA may be obtained in any number of ways which are well known to a person of ordinary skill in the art. For example, genomic DNA can be extracted and purified from suitable cells by means well-known in the art. Alternatively, mRNA can be isolated from a cell and used to prepare cDNA by reverse transcription or other means.

The term “operably linked” is used to describe a linkage between a gene sequence and a promoter or other regulatory or processing sequence such that the transcription of the gene sequence is directed by an operably linked promoter sequence, the translation of the gene sequence is directed by an operably linked translational regulatory sequence, and/or the post-translational processing of the gene sequence is directed by an operably linked processing sequence. Non-limiting examples include ATG start codons, leader sequences for export of polypeptides, ribosome binding sites and the like. For example, a promoter operably linked to a gene will provide for expression of the gene in a host cell. If a gene sequence does not contain its own promoter and ATG start codon, as in the case of the BIV pol gene sequence, these accessory sequences can be provided using techniques well known in the art.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

In the context of the present invention, the term “isolated” refers to a nucleic acid molecule, polypeptide, virus, or cell that, by the hand of man, exists apart from its native environment and is therefore not a product of nature. An isolated nucleic acid molecule or polypeptide may exist in a purified form or may exist in a non-native environment such as, for example, a recombinant host cell. An isolated virus or cell may exist in a purified form, such as in a cell culture, or may exist in a non-native environment such as, for example, a recombinant or xenogeneic organism.

The term “native” refers to a gene that is present in the genome of wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The term “consisting essentially of” as used to refer to a particular nucleic acid sequence means that the particular sequence may have up to 20 additional residues on either the 5′ or 3′ end or both, wherein the additional residues do not materially affect the basic and novel characteristics of the recited sequence.

A promoter sequence of the present invention can comprise a promoter of eukaryotic or prokaryotic origin, and will be sufficient to direct the transcription of a distally located sequence (i.e. a sequence linked to the 3′ end of the promoter sequence) in a cell. The promoter region can also include control elements for the enhancement or repression of transcription. Suitable promoters are the cytomegalovirus immediate early promoter (pCMV), the Rous Sarcoma virus long terminal repeat promoter (pRSV), and the SP6, T3, or T7 promoters. Enhancer sequences upstream from the promoter or terminator sequences downstream of the coding region optionally can be included in the vectors of the present invention to facilitate expression. Vectors of the present invention also can contain additional nucleic acid sequences, such as a polyadenylation sequence, and can encode a localization sequence, or a signal sequence, sufficient to permit a cell to efficiently and effectively process the protein expressed by the nucleic acid of the vector. Examples of preferred polyadenylation sequences are the SV40 early region polyadenylation site (C. V. Hall et al., J Molec. App. Genet. 2, 101 (1983)) and the SV40 late region polyadenylation site (S. Carswell and J. C. Alwine, Mol. Cell Biol. 9, 4248 (1989)). Such additional sequences are inserted into the vector such that they are operably linked with the promoter sequence, if transcription is desired, or additionally with the initiation and processing sequence if translation and processing are desired. Alternatively, the inserted sequences can be placed at any position in the vector.

The term “packaging construct” also sometimes referred to as a “helper construct,” refers to a DNA sequence, usually present on a plasmid but which can be incorporated into a producer cell genome, which is capable of directing expression of one or more lentiviral essential genes that provide in trans proteins required to obtain lentiviral vector particles.

A “packaging signal” or “packaging signal sequence” as used in the present invention refers to the viral RNA (or DNA) necessary for efficient packaging of viral vector RNA into infectious virions.

A gene that is “recoded” refers to a gene or genes that are altered in such a manner that the polypeptide encoded by a nucleic acid remains the same as in the unaltered sequence but the nucleic acid sequence encoding the polypeptide is changed. It is well known in the art that due to degeneracy of the genetic code, there exist multiple DNA and RNA codons which can encode the same amino acid translation product. For example, in one embodiment, a DNA sequence encoding the gag and/or pol genes of BIV is “recoded” so that the nucleotide sequence is altered but the amino acid translation sequence for the GAG and POL polypeptides remain identical to the wildtype amino acid sequence. Furthermore, it is also known that different organisms have different preferences for utilization of particular codons to synthesize an amino acid.

The term “vector” refers to a recombinant replication deficient lentiviral vector obtained when RNA encoding the viral vector construct sequences is packaged into a viral vector particle. Thus, a recombinant lentiviral vector refers to both the particle and the RNA contained therein.

A “vector construct” refers to a DNA sequence, usually in the context of a plasmid that encodes the sequence which will yield an RNA that can be packaged into an infectious viral vector particle.

In general, lentiviruses share essential features of the replication cycle, including packaging of viral RNA into a viral vector particle, infection of target cells, production of a DNA proviral copy of the RNA genome, transport of the DNA to the host nucleus, integration of the proviral DNA into the target cell chromosome, transcription of viral mRNA from the integrated DNA, expression of the gag, pol and env genes, and packaging of RNA viral transcripts into mature viral particles which are released from the host cell. The long terminal repeat (LTR) of the lentiviral genome contains cis-acting sequences important for reverse transcription, viral DNA integration and transcription and adenylation and one or more of these elements may be incorporated into the constructs of the invention. Preferably, the vector constructs of the invention comprise nucleotides corresponding to a sufficient number of nucleotides of an LTR at the 5′ end to produce a functional LTR which can direct reverse transcription of vector RNA into proviral DNA and integration of the proviral DNA into target cell genome. The constructs also can include a 3′ LTR region and include a U3 region, an R region, and optionally a U5 region.

The gag gene is the most 5′ gene on lentiviral genomes and encodes structural proteins that form the mature virus particle. The gag gene is translated to yield a precursor polypeptide that subsequently is cleaved to yield three to five structural proteins.

The pol gene encodes enzymes responsible for cleavage of lentiviral polyprotein products, reverse transcription of viral RNA and integration of proviral DNA into host chromosome.

The env gene encodes the envelope proteins which comprise the viral surface proteins of BIV and retroviruses. As used in this disclosure, the env gene includes not only natural env gene sequences but also modifications to the env gene including modifications that alter target specificity of retroviruses and lentiviruses or env genes that are used to generate pseudotyped retrovirus/lentivirus (See e.g., WO 92/14829). In general, the term “envelope surface protein gene” and “env gene” are meant to have the same meaning unless otherwise specifically indicated. The env gene can be derived from any virus, including retroviruses. The env preferably allows transduction of cells of human or other species. It may be desirable to target the recombinant virus by linkage of the envelope protein with an antibody or a particular ligand for targeting to a receptor of a particular cell-type. In such an embodiment, both the antibody or ligand combined with the envelope protein will comprise the viral surface protein gene. Preferably, the ligand is a peptide sequence genetically incorporated into the ENV protein. For example, vectors can be made target-specific by inserting a glycolipid, a protein, or a peptide. Further, targeting can be accomplished by using an antigen-binding portion of an antibody or a recombinant antibody-type molecule, such as a single chain antibody, to target the lentiviral vector. Further, the vector tropism or specific targeting can be achieved by specific modification of the vector envelope protein, such as inserting a ligand(e.g. Heparin Sulfate Proteoglycan binding motif) into the envelope. Envelopes include, but are not limited to, VSV-G envelope, LCMV envelope (Beyer et al., J Virol., 1 ;76(3):1488-95), mutant VSV-G envelope or mutant LCMV envelope. In another embodiment, the ligand may be expressed on an ecotropic envelope protein, which will serve as a scaffold to display the ligand. In a preferred embodiment, the ecotropic envelope is modified for improved vector stability (PCT Application PCT/US01/29036 (WO 02/22663)) The person skilled in the art will know of, or can readily ascertain without undue experimentation, specific methods to achieve delivery of a lentiviral vector to a specific target.

The basic genomic organization of BIV is disclosed in Garvey et al., (Virology, 175:391-409, 1990) and U.S. Pat. No. 5,380,830. The proviral LTR of BIV clone 127 is 589 nucleotides in length and is composed of U3, R and U5 elements. (See U.S. Pat. No. 5,380,830). Sequences encoding BIV and plasmids containing lentiviral genomes suitable for use in preparing the vector constructs may be readily obtained given the disclosure provided herein or from depositories and databases such as the American Type Culture Collection (ATCC), for example, ATCC Accession No. 68092 and ATCC Accession No. 68093 and GENBANK.

A “minimal packaging signal” as used in the present invention refers to a packaging signal which contains all of the sequences necessary for efficient packaging of viral vector RNA while at the same time eliminating most of the nucleotides not required for efficient packaging. In this manner, it is possible to minimize homology between the packaging signal and other viral genes or nucleic acid segments present in the recombinant constructs of the lentiviral gene transfer systems. A minimal packaging signal of BIV is shown in SEQ ID NO: 39, which contains the untranslated region (between 5′ LTR and gag start codon and the first 101 nucleotides of gag coding sequence). The person skilled in the art will readily recognize that further deletion of nucleotides may be possible, while still being able to efficiently package viral vector RNA.

Additional BIV nucleic acid sequences are reported herein, including a minimal BIV RRE, a ribosomal frame-shifting site located between the gag and pol genes, and recoded gag and pol sequences which provide reduced homology between the packaging and vector constructs of the present invention.

An “RRE” or “RRE sequence” refers to a nucleic acid sequence which interacts with the rev gene product to facilitate export of viral RNAs from the nucleus of infected cells. A “minimal RRE” or “minimal RRE sequence” refers to an RRE that consists of all of the sequences necessary for efficient export of an RNA containing the RRE from a host cell nucleus while at the same time eliminating most of the nucleotides not required for efficient RNA export. In this manner, it is possible to minimize homology between the RRE and other viral genes or nucleic acid segments present in the recombinant constructs of the lentiviral gene transfer systems. The person skilled in the art will readily recognize that further deletions may be possible which still retain RRE function. A minimal RRE of BIV is described in SEQ ID NO:40.

One, two and three construct systems comprising various components of the BIV genome on different DNA segments have been described previously. See, e.g., WO 01/44458. These systems have comprised packaging constructs which utilized BIV gag and pol genes, an env construct encoding a viral surface protein gene, and vector constructs having BIV packaging signals to package a heterologous gene of interest into recombinant lentiviral virions. The packaging signal necessary for packaging of BIV RNAs described previously was reported to span the first 200 base pairs of the BIV gag gene. The three component systems described previously comprised a BIV vector construct comprising a packaging sequence and a transgene (or heterologous gene of interest), a BIV packaging construct comprising a gag and pol gene from BIV and an env construct comprising a gene encoding a viral surface protein.

The present invention provides a three component lentiviral gene transfer system comprising a (i) packaging construct which comprises BIV gag and BIV pol genes, (ii) a viral surface protein gene construct which comprises a viral surface protein gene; and (iii) a transfer vector construct comprising a heterologous gene of interest and a BIV packaging signal and a rev gene located on one of the constructs.

The present invention also provides a four component lentiviral gene transfer system comprising (i) a packaging construct which comprises BIV gag and BIV pol genes, (ii) a viral surface protein gene construct which comprises a viral surface protein gene, (iii) a transfer vector construct comprising a heterologous gene of interest and a BIV packaging signal, and (iv) a rev expression construct comprising a rev gene.

The present invention further provides a five component lentiviral gene transfer system comprising a first packaging construct which comprises a BIV gag gene, a second packaging construct which comprises a BIV pol gene, a viral surface protein gene construct which comprises a viral surface protein gene, a transfer vector construct comprising a heterologous gene of interest and a BIV packaging signal and a rev gene construct.

The transfer vector constructs of the present invention also provide the cis-acting viral sequences necessary for a functional gene transfer vector. Such sequences include the packaging sequence (ψ), reverse transcription signals, the Primer Binding Site, integration signals, and polyadenylation sequences. The transfer vector construct can also contain a cloning site for a heterologous nucleic acid sequence to be transferred to a dividing or non-dividing cell. In a preferred embodiment, the transfer vector construct having a BIV packaging signal and a heterologous gene of interest comprises in 5′ to 3′ order: a promoter operably linked to an R region, a U5 region, a UTR region, a BIV packaging signal, an RRE, a promoter operably linked to the heterologous gene of interest, a 3′ polypurine tract a U3 region, a second R region and an optional second U5 region.

The heterologous nucleic acid sequence in the transfer vector construct is operably linked to a regulatory nucleic acid sequence. As used herein, the term “heterologous gene” or “heterologous nucleic acid sequence” refers to a sequence that originates from a foreign species, or, if from the same species, it is substantially modified in its nucleotide or amino acid sequence or level of expression, e.g., from its original form. The term also encompasses an unchanged nucleic acid sequence that is not normally expressed in a cell or is expressed at a level different from the level of expression when present as a heterologous gene. Preferably, the heterologous sequence is an open reading frame operably linked to a promoter, resulting in a chimeric gene. The heterologous nucleic acid sequence is preferably under control of either the viral LTR promoter-enhancer signals or an internal promoter, and retained signals within the lentiviral LTR can still bring about efficient integration of the vector into the host cell genome.

A wide range of promoters can be utilized to express the heterologous gene of interest, including viral or mammalian promoters. Cell or tissue specific promoters can be utilized to target expression of gene sequences in specific cell populations. Suitable mammalian and viral promoters for the present invention are available and well known in the art.

Another embodiment utilizes an inducible promoter. One example of a controlled promoter system is the Tet-On TM and Tet-Off TM systems currently available from Clontech (Palo Alto, Calif.). This promoter system allows the regulated expression of the transgene controlled by tetracycline or tetracycline derivatives, such as doxycycline. This system could be used to control the expression the heterologous gene of interest in this instant invention. Other regulatable promoter systems are described in PCT/EP01/08190 (WO 02/06463) and PCT/EP00/10430 (WO 01/30843).

Another construct of the gene transfer systems of the invention is a packaging construct comprising the BIV gag and pol genes. The BIV gag and pol genes are in different reading frames and overlap each other. In one embodiment of the invention, the gene transfer system comprises two packaging constructs, one comprising the gag gene and one comprising the pol gene. When the pol and gag genes are provided on separate constructs, the protease is encoded with the pol gene.

The BIV rev gene is made up of two exons. The first is located near the 3′ end of the central region and overlaps the 5′ end of the env gene. The second rev exon is found in the 3′ end of env but in a different reading frame. The REV protein transports intron-containing viral mRNAs, including the full length RNA encoding GAG and POL and virion packaging signals to the cytoplasm, without splicing. The REV protein functions by interacting with a cis-acting sequence of the viral genome referred to as the “Rev Response Element” or RRE.

A BIV transfer vector construct of the gene transfer systems of the present invention desirably includes a 5′ sequence comprising a promoter operably linked to a DNA segment containing R, U5, and a packaging sequence, a BIV RRE sequence, a heterologous gene of interest operably linked to another promoter and a BIV 3′ LTR. In a preferred embodiment, the packaging sequence is a BIV packaging sequence. In another preferred embodiment, the BIV packaging sequence is a minimal packaging sequence or “minimal packaging signal.”

A nucleic acid segment from a BIV genome obtainable from any strain or clone of BIV can be used in the constructs of the present invention. It will be understood that for the nucleotide sequence of the BIV genome, natural variations, which do not alter the disease pathophysiology, can exist between BIV viruses. These variations may result in deletions, substitutions, insertions, inversions or additions of one or more nucleotides as long as the function of the gene or genes is not lost. The DNA sequences encoding such variants may be created by standard cloning methods. Similarly, it will be understood by the skilled artisan that nucleotide and amino acid sequences of the present invention may readily be altered without changing the function of the corresponding nucleic acid or polypeptide or departing from the scope of the invention.

In previously described BIV viral vectors, all or part of the BIV gag gene was incorporated in the DNA segment comprising the BIV packaging sequence, which is provided on the transfer vector construct. The BIV gag gene is approximately 1,431 nucleotides (Garvey et al., Virology, 175:391-409, 1990). A desired feature of a safe and effective replication deficient recombinant lentiviral vector system will minimize homology between the packaging construct and the transfer vector construct. By minimizing homology between the constructs, the incidence of homologous recombination should be reduced, thus reducing the chance of undesired rearrangement of the constructs. In preferred embodiments the minimal packaging sequence will contain the 5′ portion of the gag gene only to the extent necessary to facilitate packaging and export of the transfer vector. Preferably, the minimal BIV packaging sequence will contain between more than the first 54 bp of the gag gene and less than about the first 200 bp of the gag gene. More preferably, the minimal BIV packaging sequence will contain more than the first 75 bp, of the gag gene sequence and preferably more than the first 90 bp, but less than the first 150 bp, preferably less than the first 125 bp, of the gag gene sequence. Most preferably, the BIV packaging sequence will contain no more than the first 101 bp of the gag gene sequence. In a preferred embodiment, the ATG start codon for the gag gene fragment that is part of the packaging sequence is mutated to prevent protein synthesis of GAG polypeptides from the DNA construct containing the minimal packaging sequence or any resulting recombinants.

In one embodiment, the transfer vector construct and optionally the packaging construct encode a minimal BIV RRE. The minimal BIV RRE has the nucleotide sequence shown in SEQ ID NO:40.

In one embodiment, the transfer vector construct of the present invention will comprise a central polypurine tract (cPPT). The cPPT may be from BIV or another lentivirus, including, for example, the cPPT of HIV. In a particularly preferred embodiment, one or more of the sequences in the U3 element of the vector construct are mutated or deleted in order to diminish or eliminate entirely U3-mediated transcription of any downstream genes. This embodiment thus provides for a self-inactivating (SIN) vector construct (Yu, et al., PNAS 83(10):3194-3198 (1986)). In such an embodiment, the heterologous gene of interest is operably linked to an internal promoter and it is also preferred that the U3 element additionally contains a sequence that enhances polyadenylation. In one particular embodiment, the polyadenylation sequence of the SV40 late polyadenylation signal upstream enhancer element is utilized.

In one embodiment, the rev and RRE comprise a rev and RRE sequence from BIV, respectively. In other embodiments, the gene transfer systems of the present invention can comprise rev and RRE segments from a lentivirus other than BIV, so long as the RRE sequence and REV can complement each other to facilitate transport of viral RNAs from the host nucleus. In one such embodiment, both the REV and RRE are derived from HIV. In a preferred embodiment, both the REV and RRE are derived from BIV.

A second construct of the gene transfer systems of the present invention is the viral surface protein gene construct. In a preferred embodiment, the viral surface protein gene is an env gene. In one embodiment, the env gene encodes the Lymphocytic Choriomeningitis Virus (LCMV) envelope or a mutant LCMV envelope. A preferred LCMV envelope is one from the LCMV-GP(WE-HPI) strain (Beyer et al., J Virol., 1 ;76(3): 1488-95). In a particularly preferred embodiment, the env gene encodes the VSV-G envelope. See, e.g., Burns et al., Proc. Natl. Acad. Sci. 90:8033-8037 (1993), Yee et al., Proc. Natl. Acad. Sci. 91:9563-9568 (1994) and U.S. Pat. No. 5,817,491, issued to Yee et al. While VSV-G protein is a desirable env gene because VSV-G confers broad host range on the recombinant virus, VSV-G can be deleterious to the producer or packaging cell. Thus, when a gene such as that for VSV-G is used, it is preferred to employ an inducible promoter system so that VSV-G expression can be regulated to minimize toxicity of the producer or packaging cell when VSV-G expression is not required. For example, the tetracycline-regulatable gene expression system of Gossen & Bujard (Proc. Natl. Acad. Sci. (1992) 89:5547-5551) can be employed to provide for inducible expression of VSV-G. The tet/VP16 transactivator may be present on a first vector and the VSV-G coding sequence may be cloned downstream from a promoter controlled by tet operator sequences on another vector.

Non-limiting examples of env genes useful for practice of the present invention include the VSV-G env, MoMLV env, Gibbon Ape Leukemia Virus (e.g., GaLV) env and env genes of the Phabdoviridae (e.g., Rabies, Mokola and Lyssa), Alphaviruses (e.g., Ross River virus, Sindbis), Paramyxovirus(e.g., Sendai), Flaviviruses (e.g., Ebola, Marburg), Retroviruses (e.g., MLV, 10A1, Xeno), Arenaviruses (e.g., LCMV or LCMV Env mutant), Thogoto viruses, Baculoviruses and Parainfluenza virus. A particular preferred env is from the LCMV-GP(WE-HPI)strain (Beyer et al., J Virol., 1:76(3):1488-95 (2002)) or VSVG.

In a further aspect of the present invention there is provided a stable packaging cell line comprising the packaging construct, envelope construct and optionally a rev gene construct of the invention. Particularly preferred packaging cell lines are such cell lines, which are capable of stably expressing at least 10 ng/ml of BIV reverse transcriptase (RT) protein. Because the packaging cell line lacks the lentiviral nucleic acid coding for packaging signal and other cis-acting elements, infectious vectors cannot be produced without a vector construct. The constructs of the packaging cell line can be episomal or integrated into the cell chromosomes.

Generally, the cell lines of the invention include a variety of the separate constructs which provide all of the functions required for packaging of recombinant vectors, such as gag, pol, env and rev, as discussed above. There is no limitation on the number of constructs which are utilized so long as they can be utilized to transform and to produce the packaging cell line to yield recombinant replication-defective lentivirus particles when a vector construct is also present in the cells.

The various constructs are introduced via transfection or infection into the packaging cell line. The packaging cell line produces viral particles that contain the vector genome. Methods for transfection or infection are well known by those of skill in the art. Thus, the packaging constructs can be introduced into human cell lines, as, for example, by calcium phosphate transfection, lipofection or electroporation, generally together with a dominant selectable marker, such as neo, DHFR, Gln synthetase or ADA, followed by selection in the presence of the appropriate drug and isolation of clones.

The packaging cell is transfected with the transfer vector construct to make a producer cell. The producer cell is cultured and it produces a plurality of the recombinant replication deficient lentiviral vector particles of the invention. The vectors are used to infect desired target cells, thereby transferring the heterologous gene of interest to the target cell.

In a preferred embodiment the producer cell line of the invention is further characterized in that it is capable of producing a lentiviral vector titer of at least 10⁵ Transducing Units/ml. Suitable host cell lines can include for example 293 cells, 293T cells, COS cells, HeLa cells, Cf2TH cells and the like.

A lentiviral vector particle may be obtained from the stable producer cell line of the invention. A method for producing a lentiviral vector particle comprises the steps of transfecting a stable packaging cell of the invention with a lentiviral vector construct, isolating and propagating the producer cell in a suitable culture medium and obtaining a lentiviral vector particle preparation from the culture medium.

The invention thus further provides a stock of recombinant lentiviral vectors obtained by harvesting the supernatant of cells transfected with the gene transfer systems of the invention.

The BIV-based recombinant lentiviral vectors of the invention can be used alone or in combination to transduce virtually any host cell or cell line. A number of target cells, including cell lines and primary cells of human and non-human origin can be infected in vitro or in vivo with the recombinant vectors of the invention. The vectors of the invention are particularly useful for infecting non-dividing primary human cell such as hematopoietic cells, for example, including stem cells, erythrocytes, neutrophils, monocytes, platelets, mast cells, eosinophils, basophils and B and T lymphocytes.

In one embodiment, the invention provides a recombinant lentivirus vector capable of infecting a dividing or non-dividing cell. The recombinant lentivirus comprises a BIV GAG protein, a BIV POL protein, a viral ENV protein, a heterologous nucleic acid sequence operably linked to a regulatory nucleic acid sequence, and cis-acting LTR nucleic acid sequences necessary for packaging, reverse transcription and integration, wherein the packaging signal is from BIV. The recombinant lentivirus of the invention is capable of infecting dividing cells as well as non-dividing cells.

The recombinant lentivirus of the invention is therefore genetically modified in such a way that some of the structural, regulatory genes of the native virus have been removed and replaced instead with a nucleic acid sequence to be delivered to a target cell. After infection of a cell by the viral vector particle, the viral vector particle releases its nucleic acid into the cell, the lentiviral vector construct is reverse transcribed and then integrated into the host cell genome. The transferred lentivirus genetic material is then transcribed and translated into proteins within the host cell.

The invention provides a method of producing a recombinant lentivirus capable of infecting a dividing or non-dividing cell by transfecting a suitable host cell with a three, four or five DNA construct system as described above and recovering the recombinant virus. In general, the genes are expressed in host eukaryotic cells from which mature recombinant virus particles or vectors are recovered.

Generally, viral supernatants are harvested using standard techniques such as filtration of supernatants at an appropriate time-point. Methods of collecting virions produced by transfected cells are described, e.g., in Riggs (Virology 218:290-295). The vector preparations can subsequently be used to infect target cells in vitro or in vivo using techniques known in the art.

The gene transfer system of the present invention can be used to provide a method of nucleic acid transfer to a dividing or non-dividing cell to provide expression of a particular nucleic acid sequence. Therefore, in another embodiment, the invention provides a method for introduction and expression of a heterologous nucleic acid sequence in a non-dividing cell by infecting the non-dividing cell with the recombinant viral vector particle of the invention and expressing the heterologous nucleic acid sequence in the non-dividing cell.

A wide variety of nucleotide sequences generally referred to as transgenes can be carried as a heterologous gene of interest by a BIV based transfer vector construct of the present invention. The nucleotide sequences should be of sufficient size to allow production of virus particles or vectors. Preferably the size of the BIV based transfer vector construct is between 1 KB to 10 KB. A non-exhaustive list of such transgenes includes sequences which encode proteins, antigens, ribozymes, antisense sequences, RNAi (Clin Exp Pharmacol Physiol 30(1-2):96-102, 2003), spliceosome-mediated RNA trans-splicing (Nat Biotechnol 17(3):246, 1999; J Invest Dermatol 115(2):332, 2000), oligonucleotides and the like.

Optionally, a selectable marker gene can be present with the transgene. Marker genes are utilized to assay for the presence of the vector, and thus, to confirm infection and integration. Marker genes can also be used to select for cells that have been transduced with the vector. The presence of a selectable marker gene ensures the growth of only those host cells which contain the vector construct. Typical selection genes encode proteins that confer resistance to antibiotics and other toxic substances, e.g. histidinol, puromycin, hygromycin, neomycin, methotrexate, etc. Some of the illustrative examples herein utilize a β-galactosidase, luciferase or enhanced green fluorescence protein (eGFP) reporter or marker system.

Antisense nucleic acids are DNA or RNA molecules that are complementary to at least a portion of a specific mRNA molecule (Weintraub, Scientific American, 262:40, 1990). In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (e.g., Marcus-Sakura, Anal.Biochem., 172:289, 1988). The antisense nucleic acid can be used to block expression of a mutant protein or a dominantly active gene product, such as amyloid precursor protein that accumulates in Alzheimer's disease. Such methods are also useful for the treatment of Huntington's disease, hereditary Parkinsonism, and other diseases. Antisense nucleic acids are also useful for the inhibition of expression of proteins associated with toxicity.

Use of an oligonucleotide to stall transcription is known as the triplex strategy since the oligomer winds around double-helical DNA, forming a three-strand helix. Therefore, these triplex compounds can be designed to recognize a unique site on a chosen gene (Maher, et al., Antisense Res. and Dev., 1(3):227, 1991; Helene, C., Anticancer Drug Design, 6(6):569, 1991).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, J.Amer.Med. Assn.,260:3030, 1988). A major advantage of this approach is that, because they are sequence-specific, only mRNAs with particular sequences are inactivated.

It may also be desirable to transfer a nucleic acid sequence that expresses a product having an antiangiogenic effect. Such compounds and the genes encoding them are known and have been described previously. Angiogenesis can be suppressed by inhibitory molecules such as α-interferon, thrombospondin-1, angiostatin and endostatin. In addition, it has been found that some tryptophanyl-tRNA synthetase derived polypeptides, shorter than the ones that occur in nature, are active in inhibiting angiogenesis, especially in ocular neovascularization (Otani, et al., PNAS 99(1):178-183 (January 2002); Wakasugi, et al., PNAS 99(1):173-177 (January 2002)). Other anti-angiogenic genes which can be used with the present invention include, but are not limited to, METH-1, METH -2, TrpRS fragments, proliferin-related protein, prolactin fragment, PEDF, vasostatin, various fragments of extracellular matrix proteins and growth factor/cytokine inhibitors. Various fragments of extracellular matrix proteins include, but are not limited to, angiostatin, endostatin, kininostatin, fibrinogen-E fragment, thrombospondin, tumstatin, canstatin, and restin. Growth factor/cytokine inhibitors include, but are not limited to, VEGF/VEGFR antagonist, soluble VEGF receptors, sFlt-1, sFlk, sNRP1, angiopoietin/tie antagonist, sTie-2, chemokines (IP-10, PF4, Gro-beta, IFN-gamma (Mig), IFNα, FGF/FGFR antagonist (sFGFR), Ephrin/Eph antagonist (sEphB4 and sephrinB2), PDGF, TGFβ and IGF-1. Delivery of such peptides or proteins via the recombinant lentiviral vectors of the present invention will be useful in regulating the growth and proliferation of vascularization associated with neo-vascular eye diseases such as age related macular degeneration, ocular complications of diabetes, rubeotic glaucoma, diabetic proliferative retinopathy, diabetic proliferative retinopathy, retinopathy of prematurity, keratitis, ischemic retinopathy and the like. By delivery of antiangiogenic factors to a tumor via the recombinant lentiviral vectors of the invention, it may be possible to suppress neovascularization and its associated pathologic effects. Examples of such inhibitory peptides can be found in PCT Application Nos. PCT/US02/05185 (WO 02/067970) and PCT/US02/23868, which are incorporated by reference in their entirety.

Soluble peptides having some or all of the amino acid sequences of Eph B receptors or ephrin B ligands also are effective inhibitors of angiogenesis, as evidenced in PCT Application No. PCT/EP01/11252 (WO 02/26827), filed Sep. 28, 2001 which is incorporated by reference in its entirety. Accordingly, the present invention will be useful for delivering nucleic acids encoding and capable of expressing such peptides to animals, particularly humans. Such peptides have exhibited an anti-angiogenic and anti-tumor effect on tumor cells.

The transgene also can comprise a nucleic acid encoding a biological response modifier. Included in this category are immunopotentiating agents including nucleic acids encoding a number of the cytokines classified as “interleukins.” Also included in this category, although not necessarily working according to the same mechanisms, are interferons, and in particular gamma interferon (γ-IFN), tumor necrosis factor (TNF) and granulocyte-macrophage-colony stimulating factor (GM-CSF). It may be desirable to deliver such nucleic acids to bone marrow cells or macrophages to treat enzymatic deficiencies or immune defects. Nucleic acids encoding growth factors, toxic peptides, ligands, receptors, or other physiologically important proteins can also be introduced into specific cells. Vectors of the invention can be used for example, to modify a host immune response, such as in graft versus host disease which occurs following allogeneic bone marrow transplantation.

Host cells and animals infected with the lentiviral vectors of the present invention further can be treated with agents such as growth factors, gangliosides, antibiotics, neurotransmitters, neurohormones, toxins, neurite promoting molecules and antimetabolites and precursors of these molecules such as the precursor of dopamine, L-DOPA.

Further, there are a number of inherited neurologic diseases in which defective genes may be replaced including: lysosomal storage diseases such as those involving β-hexosaminidase or glucocerebrosidase; deficiencies in hypoxanthine phosphoribosyl transferase activity (the “Lesch-Nyhan” syndrome“); amyloid polyneuropathies (−prealbumin); Duchenne's muscular dystrophy, and retinoblastoma, for example. In another embodiment, the invention provides methods for treating diseases of the eye. These disease include, but are not limited to, Primary open-angle glaucoma (POAG), proliferative vitreoretinopathy, diseases that involve the progressive degeneration and eventual death of photoreceptors and diseases caused by ocular neovascularization. Diseases of the eye that can be treated with the methods of the present invention include e.g., wet AMD (age related macular degeneration), diabetic proliferative retinopathy, diabetic macular edema, neovascularization due to diabetic retinopathy, non-diabetic retinopathy, branch vein occlusion, central retinal vein occlusion, retinopathy in premature infants, rubeosis iridis, neovascular glaucoma, perifoveal telangiectasis, sickle cell retinopathy, Eale's disease, retinal vasculitis, Von Hippel Lindau disease, radiation retinopathy, retinal cryoinjury, retinitis pigmentosa, retinochoroidal coloboma, corneal neovascularization due to herpes simplex keratitis, corneal ulcers, keratoplasty, pterigyia, or trauma. The methods of treating the diseases of the eye, including those noted above, comprise administering to an individual a BIV transfer vector that expresses one or more genes encoding for a gene selected from the group consisting of antiangiogenic genes, Rod-derived Cone Viability Factor (RdCVF), anti-apoptotic genes, the optineurin gene and the trabecular meshwork protein gene (TIGR). The vector is preferably delivered by direct intraocular injection in to the eye. Methods of injection into the eye are well known in the art. These include, but are not limited to, injections into the anterior or posterior chamber of the eye, e.g., into the aqueous humor or vitreous humor. Alternatively, the injection can be subretinal, e.g., by injection of aliquots (e.g., 1 to 10 microliters per aliquot) of vector-containing solution behind the retina, after which the solution is absorbed and the infectious vector particles infect local cells of the ocular tissues. Such administration can comprise either a single injection, multiple injections administered on the same day, single injections administered over a period of weeks or months, or multiple injections administered over a period of weeks or months.

Rod-derived Cone Viability Factor (RdCVF) has been found to be a cone protective factor (PCT Application No. PCT/EP02/03810 (WO 02/081513).In a preferred embodiment, a BIV vector of the present invention contains at least one nucleic acid sequence encoding for an RdCVF polypeptide as encoded in SEQ ID NO:61, SEQ ID NO:63, SEQ ID NO:65 or SEQ ID NO:67. Thus, in another embodiment, the invention provides a method for demonstrating a cone protective effect either in vitro or in vivo by transferring an RdCVF gene to a cell using the BIV vectors system of the present invention. In a preferred embodiment, the BIV vector encodes for a human RdCVF1 and/or RdCVF2.

A BIV vector expressing RdCVF may be used to treat various diseases related to the eye. Thus, in another preferred embodiment, the invention provides a method for treating a human afflicted with a retinal dystrophy such as retinitus pigmentosa, age-related macular degeneration, Bardet-Biedel syndrome, Bassen-kornzweig syndrome, best disease, choroidema, gyrate atrophy, congenital amourosis, refsun syndrome, stargardt disease and Usher syndrome.

For diseases due to deficiency of a protein product, gene transfer could introduce a normal gene into the affected tissues for replacement therapy. This gene delivery technology can also be used to create transgenic animals.

In one embodiment, the BIV vectors may be used as a tool to identify gene function. Accordingly, the vectors may be used to transfer an expression cassette into cells in vitro to over-express a specific gene or diminish expression of a specific gene. By observing the phenotype associated with up or down-regulation of expression of a specific gene, it is possible to determine the function of that gene. This information has great value in determining if the gene product is a valid target for the development of pharmaceuticals.

Methods for diminishing expression of specific genes are well known to those skilled in the art and include expression of a ribozyme, antisense oligonucleotide, or RNAi directed at the mRNA of the gene whose expression is to be diminished. An alternative strategy to diminish expression would be to trans-splice a 3′ exon sequence that encodes a stop codon. A second alternative strategy would be to express a protein that functions as a dominant negative to the protein whose function is to be diminished.

In a further embodiment, the BIV vectors may be used to identify gene function in vivo. Accordingly, the vectors may be used to transfer an expression cassette into cells in vivo to over-express a specific gene or diminish expression of a specific gene. This may be accomplished by administering the vector to an animal via direct injection into a tissue or body cavity or by administering the vector directly into the circulation. Alternatively, the vector could be administered to cells in vitro and then the cells could be injected or implanted into an animal. Appropriate injection sites or implant devices are known to those skilled in the art. Identification of gene function in vivo would also have great value in determining if the gene product is a valid target for the development of pharmaceuticals.

In another embodiment, the vector may be used to screen libraries for specific functions to clone the gene for that function. In this embodiment, a library, which may be a cDNA library, would be encoded in the transfer vector construct. The transfer vector construct would then be used to generate vector and the vector applied to cells in vitro or in vivo. The cells that exhibited the desired function would be isolated. The gene of interest could be readily recovered from the BIV vector integrant. Methods of using integrating vectors for gene cloning are well known to those skilled in the art. The technology described herein has significant advantages over retroviral vector systems used for gene cloning in that the BIV vectors will efficiently transduce cells in vivo, whereas retroviral vectors will not.

In another embodiment, the BIV vectors may be used to establish a strong immune response to specific proteins. Administration of BIV vectors to animals, particularly via intravenous injection, results in efficient transduction of cells in the spleen. In this setting, expression of the encoded transgene can result in a strong immune response to the expressed protein. This technology may be used to improve the efficiency of generating antibodies, including monoclonal antibodies, for research, diagnostic, or therapeutic purposes. The technology may also be applied directly in humans for immunotherapeutic purposes.

The preferred vectors of the present invention are derived from BIV. Native BIV nucleic acid can be isolated from cells infected with the virus, and vectors prepared therefrom. For example, cDNA can be produced from BIV RNA by reverse transcriptase, using methods known in the art. Double-stranded BIV cDNA then can be produced and cloned into a cloning vector, such as a bacterial cloning vector. Any cloning vector, such as bacterial, yeast or eukaryotic vectors, known and used by those skilled in the art, can be used.

Large amounts of the nucleic acids comprising the DNA constructs of the present invention can be produced by (a) replication in a suitable host or transgenic animals or (b) chemical synthesis using techniques well known in the art. Constructs prepared for introduction into a prokaryotic or eukaryotic host can comprise a replication system recognized by the host, including the intended polynucleotide fragment encoding the desired polypeptide, and preferably also will include transcription and translational initiation regulatory sequences operably linked to the polypeptide encoding segment. These can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals also can be included where appropriate which allow the protein to cross and/or lodge in cell membranes, and thus attain its functional topology, or be secreted from the cell. Such vectors may be prepared by means of standard recombinant techniques well known in the art.

In accordance with the above, the following embodiments of the invention are contemplated and form part of the invention.

1. A recombinant lentiviral gene transfer system, comprising:
(a) (i) a packaging construct comprising a DNA segment comprising a promoter operably linked to a BIV gag gene and a BIV pol gene, or (ii) a first packaging construct comprising a DNA segment comprising a first promoter operably linked to a DNA segment comprising a BIV gag gene and a second packaging construct comprising a DNA segment comprising a second promoter operably linked to a DNA segment comprising a BIV pol gene;
(b) a viral surface protein gene construct comprising a DNA segment comprising a promoter operably linked to a viral surface protein gene;
(c) a transfer vector construct comprising a DNA segment comprising a promoter operably linked to a first R region, a U5 region, a UTR region, a BIV packaging sequence, an RRE sequence, a promoter operably linked to a heterologous gene of interest, a 3′ polypurine tract region, a U3 region, and a second R region; and
(d) (i) a rev gene located on one of the packaging, viral surface protein gene, and transfer vector constructs or (ii) a rev construct comprising a DNA segment comprising a promoter operably linked to a rev gene.

2. The gene transfer system of 1, comprising a packaging construct comprising a DNA segment comprising a promoter operably linked to a BIV gag gene and a BIV pol gene.

3. The gene transfer system of 1, comprising a first packaging construct comprising a DNA segment comprising a first promoter operably linked to a DNA segment comprising a BIV gag gene and a second packaging construct comprising a DNA segment comprising a second promoter operably linked to a DNA segment comprising a BIV pol gene.

4. The gene transfer system of 2, wherein the packaging construct further comprises an RRE sequence.

5. The gene transfer system of 3, wherein at least one of the packaging constructs further comprises an RRE sequence.

6. The gene transfer system of 1, wherein the rev gene and RRE sequence are from BIV.

7. The gene transfer system of 2, wherein the gag gene comprises a recoded nucleotide sequence.

8. The gene transfer system of 2, wherein the gag and pol genes each comprise a recoded nucleotide sequence.

9. The gene transfer system of 2, wherein the pol gene comprises a recoded nucleotide sequence.

10. The gene transfer system of 3, wherein the gag gene comprises a recoded nucleotide sequence.

11. The gene transfer system of 3, wherein the pol gene comprises a recoded nucleotide sequence.

12. The gene transfer system of 11, wherein the pol gene comprises an ATG start codon at the 5′ end.

13. The gene transfer system of 1, wherein the protease region of the pol gene is mutated in the three amino acid motif of the catalytic center of the protease and wherein the mutated protease is less toxic to host cells when compared to a non-mutated BIV protease.

14. The gene transfer system of 13, wherein wherein the protease region encodes a Thr to Ser mutation at amino acid 26 of the protease polypeptide.

15. The gene transfer system of 1, wherein the BIV packaging sequence comprises no more than the first 101 base pairs of the BIV gag gene open reading frame sequence.

16. The gene transfer system of 15, wherein the packaging sequence consists essentially of the nucleotide sequence of SEQ ID NO:39.

17. The gene transfer system of 1, wherein the transfer vector construct comprises a DNA segment comprising a promoter operably linked to a first R region, a U5 region, a UTR region, a BIV packaging sequence, an RRE sequence, a promoter operably linked to a heterologous gene of interest, a 3′ polypurine tract region, a U3 region, a second R region, and a second U5 region.

18. The gene transfer system of 2, wherein the packaging construct further comprises the rev gene.

19. The gene transfer system of 1, wherein the viral surface protein gene construct comprises an env gene.

20. The gene transfer system of 19, wherein the env gene is selected from the group consisting of VSV-G env, LCMV env, LCMV-GP(WE-HPI) env, MoMLV env, Gibbon Ape Leukemia Virus (GaLV) env, an env gene from a member of the Pbabdoviridae, an Alphavirus env gene, a Paramyxovirus env gene, a Flavivirus env gene, a Retrovirus env gene, an Arenavirus env gene, a Parainfluenza virus env gene, a Thogoto virus env gene, and a Baculovirus env gene.

21. The gene transfer system of 1, wherein the viral surface protein gene encodes VSV-G env.

22. The gene transfer system of 1, comprising a rev gene located on one of the packaging, viral surface protein gene, and transfer vector constructs.

23. The gene transfer system of 1, comprising a rev construct comprising a DNA segment comprising a promoter operably linked to a rev gene.

24. The gene transfer system of 6, wherein the rev gene does not include the native BIV rev intron.

25. The gene transfer system of 24, wherein the rev gene comprises SEQ ID NO:10.

26. The gene transfer system of 22, comprising an EF-1 promoter operably linked to the rev gene.

27. The gene transfer system of 23, wherein the promoter operably linked to the rev gene is the EF-1 promoter.

28. The gene transfer system of 6, wherein the RRE sequence consists essentially of the nucleic acid sequence of SEQ ID NO:40.

29. The gene transfer system of 1, wherein at least two of the promoters are the same.

30. The gene transfer system of 1, wherein all of the promoters are different.

31. The gene transfer system of 1, wherein at least one of the promoters is a regulatable promoter.

32. The gene transfer system of 1, which does not contain a cPPT.

33. The gene transfer system of 1, wherein the transfer vector construct further comprises a cPPT.

34. The gene transfer system of 33, wherein the cPPT is the cPPT from Human Immunodeficiency Virus.

35. The gene transfer system of 33, wherein the cPPT is a BIV cPPT.

36. The gene transfer system of 35, wherein the cPPT consists essentially of 535 base pairs corresponding to the nucleotides from base pairs 4758 to 5293 inclusive of SEQ ID NO:1.

37. The gene transfer system of 1, wherein the U3 region comprises an enhancer of polyadenylation.

38. The gene transfer system of 37, wherein the enhancer of polyadenylation consists essentially of the SV40 late polyadenylation enhancer element.

39. The gene transfer system of 1, which does not encode at least one of the vif, vpw, vpy, or tat genes of BIV.

40. The gene transfer system of 1, which does not encode the vif, vpw, vpy, tmx, and tat genes of BIV.

41. The gene transfer system of 1, wherein one or more nucleotides in the U3 region are altered or deleted such that U3 mediated transcription is diminished or abolished.

42. The gene transfer system of 1, comprising a woodchuck hepatitis virus regulatory response element operably linked to the heterologous gene of interest.

43. The gene transfer system of 1, wherein the heterologous gene of interest encodes a polypeptide selected from the group consisting of: T2-TrpRS, an Eph B receptor, an ephrin B ligand, a Fibrinogen E fragment, a soluble receptor for VEGF, angiostatin, endostatin, optineurin, trabecular meshwork protein, a Rod-derived Cone Viability Factor (RdCVF) and an anti-apoptotic gene product.

44. The gene transfer system of 1, wherein the heterologous gene of interest encodes an RdCVF polypeptide selected from the group consisting of: SEQ ID NO: 61, SEQ ID NO:63, SEQ ID NO:65 and SEQ ID NO:67.

45. A producer cell comprising the gene transfer system of any one of 1-44.

46. The producer cell of 45, wherein the gene transfer system is stably integrated into the producer cell's genome.

47. The producer cell of 45, wherein the gene transfer system is transiently transfected into the producer cell.

48. A method of producing replication-defective lentiviral vector particles, comprising:
(a) growing the producer cell of 45 in cell culture media under cell culture conditions sufficient to allow production of replication-defective lentiviral vector particles by the cell; and
(b) collecting said replication-defective lentiviral vector particles from the media.

49. A method according to 48, which further comprises adding a histone deacetylase inhibitor to the media.

50. A method according to 49, wherein the histone deacetylase inhibitor is butyric acid.

51. A replication-defective lentiviral vector particle produced according to the method of 48.

52. A method of treating or preventing a disease in an animal which has or is at risk of contracting said disease, comprising infecting one or more cells of the animal with a replication deficient recombinant lentiviral vector particle according to 51, wherein the heterologous gene of interest encodes a therapeutic product that is effective in treating or preventing said disease.

53. The method of 52, wherein the animal is a human.

54. The method of 52, wherein the one or more cells are ocular cells.

55. The method of 54, wherein the disease is selected from the group consisting of: ocular neovascularization, wet AMD (age related macular degeneration), diabetic proliferative retinopathy, non-diabetic retinopathy, diabetic macular edema, branch vein occlusion, central retinal vein occlusion, retinopathy in premature infants, rubeosis iridis, neovascular glaucoma, perifoveal telangiectasis, sickle cell retinopathy, Eale's disease, retinal vasculitis, Von Hippel Lindau disease, radiation retinopathy, retinal cryoinjury, retinitis pigmentosa, retinochoroidal coloboma, corneal neovascularization due to herpes simplex keratitis, corneal ulcers, keratoplasty, pterigyia, or traumaretinal dystrophy, pathological aging, retinitus pigmentosa, Bardet-Biedel syndrome, Bassen-kornzweig syndrome, Best disease, choroidema, gyrate atrophy, congenital amourosis, Refsun syndrome, Stargardt disease and Usher syndrome.

56. The method of 55, wherein the therapeutic product is selected from the group consisting of: T2-TrpRS, an Eph B receptor, an ephrin B ligand, a Fibrinogen E fragment, a soluble receptor for VEGF, angiostatin, endostatin, optineurin, trabecular meshwork protein, a Rod-derived Cone Viability Factor (Rdcvf) and an anti-apoptotic gene product.

57. The method of 55, wherein the therapeutic product is an Rdcvf polypeptide selected from the group consisting of: SEQ ID NO: 61, SEQ ID NO:63, SEQ ID NO:65 and SEQ ID NO:67.

58. The method of 52, wherein the disease is selected from the group consisting of: cancer, graft versus host disease associated with allogeneic bone marrow transplant, and a neurologic disease.

59. The method of 52, wherein the one or more cells are infected in vivo.

60. The method of 52, wherein the one or more cells are infected in vitro.

61. A method of transducing cells in vitro with a recombinant lentiviral vector particle, comprising contacting the cells with the recombinant lentiviral vector particle according to 51, whereby the cells are transduced.

62. A method of transducing cells in vivo with a recombinant lentiviral vector particle, comprising contacting the cells with the recombinant lentiviral vector particle according to 51, whereby the cells are transduced.

63. A method of expressing a heterologous gene of interest in a cell which comprises transducing the cell with the recombinant lentiviral vector particle according to 51, whereby the heterologous gene of interest is expressed in the cell.

64. A packaging cell, comprising:
(a) (i) a packaging construct comprising a DNA segment comprising a promoter operably linked to a BIV gag gene and a BIV pol gene, or (ii) a first packaging construct comprising a DNA segment comprising a first promoter operably linked to a DNA segment comprising a BIV gag gene and a second packaging construct comprising a DNA segment comprising a second promoter operably linked to a DNA segment comprising a BIV pol gene;
(b) a viral surface protein gene construct comprising a DNA segment comprising a promoter operably linked to a viral surface protein gene; and
(c) (i) a rev gene located on one of the packaging, viral surface protein gene, and transfer vector constructs or (ii) a rev construct comprising a DNA segment comprising a promoter operably linked to a rev gene.

65. The packaging cell of 64, comprising a packaging construct comprising a DNA segment comprising a promoter operably linked to a BIV gag gene and a BIV pol gene.

66. The packaging cell of 64, comprising a first packaging construct comprising a DNA segment comprising a first promoter operably linked to a DNA segment comprising a BIV gag gene and a second packaging construct comprising a DNA segment comprising a second promoter operably linked to a DNA segment comprising a BIV pol gene.

67. The packaging cell of 65, wherein the gag gene comprises a recoded nucleotide sequence.

68. The packaging cell of 65, wherein the gag and pol genes each comprise a recoded nucleotide sequence.

69. The packaging cell of 65, wherein the pol gene comprises a recoded nucleotide sequence.

70. The packaging cell of 66, wherein the gag gene comprises a recoded nucleotide sequence.

71. The packaging cell of 66, wherein the pol gene comprises a recoded nucleotide sequence.

72. The packaging cell of 64, wherein the protease region of the pol gene is mutated in the three amino acid motif of the catalytic center of the protease and wherein the mutated protease is less toxic to host cells when compared to a non-mutated BIV protease.

73. The packaging cell of 72, wherein wherein the protease region encodes a Thr to Ser mutation at amino acid 26 of the protease polypeptide.

74. The packaging cell of 64, wherein the viral surface protein gene construct comprises an env gene.

75. The packaging cell of 74, wherein the env gene is selected from the group consisting of VSV-G env, LCMV env, LCMV-GP(WE-HPI) env, MoMLV env, Gibbon Ape Leukemia Virus (GaLV) env, an env gene from a member of the Phabdoviridae, an Alphavirus env gene, a Paramyxovirus env gene, a Flavivirus env gene, a Retrovirus env gene, an Arenavirus env gene and a Parainfluenza virus env gene.

76. The packaging cell of 64, wherein the viral surface protein gene encodes VSV-G env.

77. The packaging cell of 64, comprising a rev gene located on one of the packaging, viral surface protein gene, and transfer vector constructs.

78. The packaging cell of 64, comprising a rev construct comprising a DNA segment comprising a promoter operably linked to a rev gene.

79. The packaging cell of 64, wherein the rev gene is from BIV but does not include the native BIV rev intron.

80. The packaging cell of 79, wherein the rev gene comprises SEQ ID NO:10.

81. The packaging cell of 77, comprising an EF-1 promoter operably linked to the rev gene.

82. The packaging cell of 78, wherein the promoter operably linked to the rev gene is the EF-1 promoter.

83. The packaging cell of 64, wherein at least two of the promoters are the same.

84. The packaging cell of 64, wherein all of the promoters are different.

85. The packaging cell of 64, wherein the cell is selected from the group consisting of a 293 cell, a 293T cell, a COS cell, a HeLa cell, and a Cf2TH cell.

86. An isolated BIV POL protein, comprising an amino acid sequence at least 90% identical to the amino acid sequence shown in SEQ ID NO:51.

87. The isolated BIV POL protein of 86, comprising SEQ ID NO:51.

88. The isolated BIV POL protein of 86, comprising a methionine at the N-terminus of said POL protein.

89. An isolated nucleic acid molecule comprising a nucleotide sequence encoding the BIV POL protein of any one of 86-88.

90. An isolated nucleic acid molecule comprising a nucleotide sequence encoding the BIV POL protein of 87, wherein said nucleotide sequence consists essentially of SEQ ID NO:50.

91. An isolated nucleic acid molecule comprising a nucleotide sequence encoding the BIV POL protein of 88, wherein said nucleotide sequence consists essentially of SEQ ID NO:53.

92. An isolated nucleic acid molecule comprising a minimal BIV packaging sequence, wherein said minimal BIV packaging sequence is at least 90% identical to the nucleotide sequence set forth in SEQ ID NO:39.

93. The isolated nucleic acid molecule of 92, wherein the minimal BIV packaging sequence consists essentially of the nucleotide sequence set forth in SEQ ID NO:39.

94. An isolated nucleic acid molecule comprising a nucleotide sequence encoding a BIV REV protein, wherein said nucleotide sequence encodes an amino acid sequence at least 90% identical to the amino acid sequence encoded by the nucleotide sequence set forth in SEQ ID NO:10.

95. The isolated nucleic acid molecule of 94, wherein the nucleotide sequence encoding the BIV REV protein encodes the same amino acid sequence encoded by the nucleotide sequence set forth in SEQ ID NO:10.

96. The isolated nucleic acid molecule of 94, wherein the nucleotide sequence is at least 90% identical to the nucleotide sequence set forth in SEQ ID NO:10.

97. The isolated nucleic acid molecule of 94, wherein the nucleotide sequence consists essentially of the nucleotide sequence set forth in SEQ ID NO:10.

98. An isolated nucleic acid molecule comprising a minimal BIV RRE sequence, wherein said minimal BIV RRE sequence is at least 90% identical to the nucleotide sequence set forth in SEQ ID NO:40.

99. The isolated nucleic acid molecule of 98, wherein the minimal BIV RRE sequence consists essentially of the nucleotide sequence set forth in SEQ ID NO:40.

The present invention is further detailed in the following Examples, which are offered by way of illustration and are not intended to limit the invention in any manner. Standard techniques well known in the art or the techniques specifically described below are utilized.

### EXAMPLES

### Example 1

SEQ ID NO:1 shows the DNA sequence of bovine immunodeficiency virus provirus.

### Example 2

### Plasmid Construction

The packaging construct was created by ligating the necessary constructs of BIV into the mammalian expression plasmid, pCI (Promega, Madison, Wis.). The major splice donor (MSD) site and the coding sequence for gag and pol was isolated as a 4485 base pair BspEI-BstUI fragment from the BIV provirus (Garvey, et al. Virology. April 1990;175(2):391-409, Genbank Accession No. NC_{—}001413and M32690). This fragment was blunt ended by Klenow treatment, and ligated to pCI linearized with EcoRI and also blunt ended by Klenow treatment to create pCIigp. Next, PCR amplification of the BIV provirus with primers RRE65′NotI (5′-AAAGCGGCCGCTCCGGTGGATTCTTGTAAAGG-3′) (SEQ ID NO:2) and RRE63′NotI (5′-AAAGCGGCCGCGGCGCCTCCAAGTATGAAACTC-3′) (SEQ ID NO:3) created the minimal RRE fragment. This 344 base pair PCR fragment was digested with NotI and ligated to pCIigp also digested with NotI and phosphatase (CIP) treated. The plasmid created is named pCIigpRRE, and was used in the four component system. Finally, a contiguous coding sequence for rev, with the two exons fused, was created by two different methods, RT-PCR and PCR SOEing, as described below.

The rev sequence used in the four component system was created by RT-PCR. In brief, 239 T cells seeded in 10-cm culture dish were transfected with 20 ug of pBH2 plasmid (Berkowitz et. al., 2001), using ProFectin Mammalian Transfection System (Promega). Forty-eight hours after transfection, the cells were harvested and total RNA was purified with Trizol Reagent (Invitrogen). The RT-PCR were performed with GeneRacer Kit (Invitrogen), following the manufacturer's instruction. 5 ug of total RNA were used to synthesize cDNAs. The rev cDNA sequence was amplified using primers: Rev15Afl3 (5′-GGACGCGTCGACTCTAGATCTAGGAATCAACTATGG-3′) (SEQ ID NO:4) and Rev23Agel2 (5′-TTTACCGGTCGCGAGCTTAGCTTACAATCTACTGAGAACC-3′) (SEQ ID NO:5). The PCR reactions were carried out under the following conditions: 94° C. for 1 min.; 25 cycles of 94° C. for 1 min, 54° C. for 1 min. and 72° C. for 1 min.; and 72° C. for additional 10 min. The 0.7 kb rev cDNA fragment was detected on a 1% agarose gel, and then subsequently cloned into pCR4-TOPO vector, following the instruction of TOPO TA Cloning Kit (Invitrogen). Two clones with rev cDNA insert were identified. The orientation of the rev inserts were determined by restriction enzyme digestion. After rev cDNA clones were confirmed by automatic sequencing, the rev gene was subcloned into pTracerA plasmid (Invitrogen) for expression in mammalian cells. The rev sequence was inserted between PmeI and NotI sites under the control of EF-1á promoter, creating pTracerARev. Both pCIigpRRE and pTracerARev were DNA sequenced to confirm the integrity of the constructs. The rev coding sequence ligated into pCIigpRRE for the three construct system was created by PCR SOEing (Splicing by Overlap Extension). The first exon of rev was amplified by PCR, from the BIV provirus, using primers Rev155868 (5′-GTTCTAGATGGCTGGCTTTTCTGG-3′) (SEQ ID NO:6) and Rev13(new)(5′-GAGAATCGTTATTGATCCATGTTTG-3′) (SEQ ID NO:7). The second exon was also amplified from the provirus, using primers Rev25(new)(5′-GGATCAATAACGATTCTCCTAGGTATGT-3′) (SEQ ID NO:8) and Rev237526(5′-TTACTAGTGGTTATTTTGTTCCCTGG-3′) (SEQ ID NO:9). The two products were mixed in equimolar amounts and amplified using primers Rev155868 and Rev237526. The final 698 base pair product was digested with XbaI and SpeI and ligated to pCIigpRRE digested with XbaI. The resulting plasmid is named pBIVminipack and contains only the CMV immediate early promoter driving the BIV gag/pol coding sequence, followed by the fused coding sequence for rev, the minimal RRE, and finally an SV40 polyadenylation signal. There still remains the MSD site upstream of the start of gag, and the splice acceptor (SA) site for rev. The packaging constructs were then subjected to DNA sequencing to confirm the integrity of the construct. The sequence of the rev gene without the intervening intron is shown in SEQ ID NO:10. In one embodiment, the invention provides a gene transfer system wherein the rev gene does not contain the native BIV intervening intron and in another embodiment the rev gene does not contain any intervening intron.

The transfer vector construct pBIVminivec was derived from pBC4MGppt, which has previously been described (Berkowitz et. al., 2001). To facilitate the cloning, the entire BIV transfer vector coding sequence was cloned into the expression plasmid pBS II KS+ (Stratagene, La Jolla, Calif.) by digesting pBC4MGppt with BspMI and ligated to pBS II KS+ previously digested with HincII as a blunt end ligation to create the plasmid pBSV4MGppt. The plasmid pBSV4MGppt was digested with BglII and EcoNI, Klenow treated and re-ligated to remove a 297 bp fragment of the gag gene to create the plasmid pBSV4MGpptΔGAG. Due to the lack of unique and convenient restriction sites immediately following the enhanced green fluorescent protein (eGFP) reporter gene, a unique PstI site was incorporated using the primers WPRES (5′-GAGCTGTACAAGTAAAGCGGCCAACCCTCCTGCAG-AAACTCCTTTGGG-3′) (SEQ ID NO:11) and WPRE3 (5′-GGAACAAAAGCTGGGTACCGGGCCCCCCC-3′) (SEQ ID NO:12) to create the plasmid pBSV4MGpptΔGAG PstI. The Woodchuck hepatitis post-transcriptional regulatory element (PRE) was then cloned into the backbone, pBSV4MGpptΔGAG PstI, which was previously digested with PstI, treated with Klenow and ligated to the PRE fragment to create the plasmid pBSV4MGpptΔGAG PRE. The plasmid pBSV4MGpptΔGAG PRE was further modified by removing all of the putative rev response element (RRE), which is about 778 bp in the original BIV transfer vector (Berkowitz et. al., 2001) and replacing it with a 312 bp fragment of the RRE which we found to be fully responsible for RRE function. First, the plasmid pBSV4MGpptΔGAG PRE was digested with Kasl and BbvCI. This region was then PCR amplified with the primers RRE1 (5′-GTTGGCGCCCAACGTGGGGCTCGAGTAAGAGAG-3′) (SEQ ID NO:13), RRE2 (5′-AGATCTGAATTCTAAGTG-ACCTATTTC-3′) (SEQ ID NO:14) RRE3 (5′-GAATTCAGATCTTATG-GGAATGAAAGACC-3′) (SEQ ID NO: 15) and RRE4 (5′-AACTGCTGAGGGCGGGACCGCATCTGG-3′) (SEQ ID NO:16). RRE1 and RRE2 amplified the upstream region of the putative RRE and primers RRE3 and RRE4 amplified the downstream region of the putative RRE. The products were then mixed in equal molar ratios and amplified with primers RRE1 and RRE4. The final product incorporated the KasI and BbvCI sites with the entire putative RRE deleted. Furthermore, there were unique EcoRI and BglII sites constructed to create junction sites between primers RRE2 and RRE3 for annealing purposes of the final product, but primarily for subsequent cloning of various regions of the RRE. This PCR strategy created the plasmid pBSV4MGpptΔGAGΔRRE PRE. The putative RRE was then PCT amplified with 7 sets of primers (Table 1) in various regions all encoding a 5′ EcoRI site and a 3′ BglII site to be cloned into the backbone, pBSV4MGpptΔGAGΔRRE PRE. Once created, each fragment was digested with EcoRI and BglII and cloned as previously described (Table 1). The vector construct containing RRE6 was named pBIVminivec.

The plasmid containing the viral surface protein gene construct used in the present examples, which express VSV-G has been described previously (Burns et al., 1993, PNAS. USA 90:8033-8037).

TABLE 1

| | Base | | Transduction |
|---|---|---|---|
| Construct | Pairs | Primer Sequence | Efficiency |
| +contol | 6783-7561 | N/A | 100% |
| pBSV4MGpptAGAG | | | |
| 1. pBSV4MGpptΔGAGRRE1 | 6783-7030 | 5′ ggcgaattcgatctaggaaaaaattttccg-3′ | 1% |
| | | SEQ ID NO:17 | |
| | | SEQ ID NO:18 | |
| | | 3′ ggaagatctccacaaacccatagctgg-5′ | |
| 2. pBSV4MGpptΔGAGRRE2 | 7005-7290 | 5′ cccgaattcaaaggtccccagc-3′ | 65% |
| | | SEQ ID NO:19 | |
| | | SEQ ID NO:20 | |
| | | 3′ ggaagatctctctatggtgtaggac-5′ | |
| 3. pBSV4MGpptΔGAGRRE3 | 7288-7561 | 5′ ccggaattcgagtttcatacttggag-3′ | 9% |
| | | SEQ ID NO:21 | |
| | | SEQ ID NO:22 | |
| | | 3′ ggaagatcttgcactaaatggtc-5′ | |
| 4. pBSV4MGpptΔGAGRRE4 | 6908-7181 | 5′ ccggaattccctaatactatgcc-3′ | 38% |
| | | SEQ ID NO:23 | |
| | | SEQ ID NO:24 | |
| | | 3′ ggaagatctcttagccgtcgtgtgc-5′ | |
| 5. pBSV4MGpptΔGAGRRE5 | 7192-7431 | 5′ ggcgaattcgggttgtgcaaaatgtg-3′ | 3% |
| | | SEQ ID NO:25 | |
| | | SEQ ID NO:26 | |
| | | 3′ cctagatctcattccaagttttgct-5′ | |
| 6. pBSV4MGpptΔGAGRRE6 | 6993-7304 | 5′ ccggaattcgtggattcttgtaaagg-3′ | 106% |
| | | SEQ ID NO:27 | |
| | | SEQ ID NO:28 | |
| | | 3′ ggaagatctctccaagtatgaaactc-5′ | |
| 7. pBSV4MGpptΔGAGRRE7 | 7048-7345 | 5′ ccagaattccaccaccatccctcc-3′ | 98% |
| | | SEQ ID NO:29 | |
| | | SEQ ID NO:30 | |
| | | 3′ ggaagatctcaaccaaagaatact-5′ | |

To further delete the remaining 212 bp gag sequence in the minimal vector, the construct pBSV4MGpptΔGAGΔRRE PRE (Designated pBvΔRRE for simplicity) was used as the template for the PCR reactions to make deletions in the gag sequence to determine the location of the packaging signal. The construct pBvΔRRE, which was described above, was used to delete 184 bp of GAG by digesting with Cla I and Hind III, treating with klenow, and then religating. This cloning strategy resulted in a construct containing 28 bp of Gag coding sequence, creating the plasmid pBv28ΔRRE. Next we created a vector construct containing 54 bp of gag sequence. First, the template pBvΔRRE was digested with KasI and EcoRI and alkaline phosphatase treated. Gag coding region was amplified using the primers NRS1 (5′AACAGTTGGCGCCCAACGTGGGGCTC-3′) (SEQ ID NO:31), NRS2 (5′ATGCATCACGTGGGGTGTCACCCTAACCTTACGAA-3′) (SEQ ID NO:32), NRS3 (5′CACGTGATGCATCGATCTAAAAGACAGATTGGC-3′) (SEQ ID NO:33), and NRS4 (5′CATAAGATCTGAATTCAATGATCTAAGTG-3′) (SEQ ID NO:34). NRS1 and NRS2 were used to amplify the 5′ region of the Gag start codon (ATG) to base pair 54 of Gag. NRS3 and NRS4 amplified 3′ of the stop codon of Gag through the BIV cPPT. The products were then mixed in equal molar ratios and amplified with primers NRS1 and NRS4. The final product incorporated the KasI and EcoRI sites, deleting the last 158 bps of Gag within the template resulting in a construct containing 54 bp of Gag coding sequence. Furthermore, there were unique Nsi I and PMII sites incorporated to create junction sites between primers NRS2 and NRS3 for annealing and screening of the final product. This PCR strategy created the plasmid pBv54ΔRRE. The same PCR strategy was implemented to create the construct pBv104ΔRRE. NRS1 and NRS4 were used as external primers and the new internal primers are NRS32 (5′ ATGCATCACGTGATTCTAATGGCCCATTGAAGATTC-3′) (SEQ ID NO:35), and NRS33 (5′ CACGTGATGCATCGATCTAAAAGACAGATTGGC-3′) (SEQ ID NO:36). NRS1 and NRS32 were used to amplify the 5′ region of the Gag start codon (ATG) to base pair 101 of Gag. NRS33 and NRS4 amplified 3′ of the stop codon of Gag through the BIV cPPT. The products were then mixed in equal molar ratios and amplified with NRS1 and NRS4. The final product incorporated the KasI and EcoRI sites, deleting the last 111 bps of Gag within the template pBvΔ104RRE. And, as described above, there were unique Nsi I and PmlI sites incorporated to create junction sites between NRS32 and NRS33 for annealing and screening of the final product. Finally, all three constructs, pBv28ΔRRE, pBv54 RRE, and pBv104ΔRRE were digested with EcoRI and Bgl II, alkaline phosphatase treated and then the minimal RRE6, as described above, was cloned in as an EcoRI and Bgl II fragment to create the plasmids pBv28, pBv54, and pBv101, respectively. All these transfer vector constructs were subjected to DNA sequencing to confirm the integrity of the construct. pBv101 was designated as pBIVfinalvecATG.

To mutate the start codon of BIV gag, we used the QuickChange strategy (Stratagene, LaJolla, Calif.). pBSIIKS+ (Stratagene, LaJolla, Calif.) was digested with NotI and HindIII, and alkaline phosphatase treated (CIP). Next, pBIVminivec was digested with NotI and HindIII. The NotI to HindIII fragment from pBIVminivec was subcloned into the pBSIIKS+ backbone. The primers used for the QuickChange reaction are:

KOATG Forward

SEQ ID NO:37

(5′-GCGTGTTTTCCCCGGGGTGAAGAGAAGGGAG-3′)
and
KOATG Reverse

SEQ ID NO:38

(5′-CTCCCTTCTCTTCACCCCGGGGAAAACACGC-3′).

This plasmid was called pBSIIKS+NHΔATG. The product was then subjected to DNA sequencing to confirm the integrity of the product. pBSIIKS+NHΔATG was then digested with NotI and HindIII and then cloned back into pBIVfinalvecATG. The final construct created was designated pBIVfinalvec and had the ATG of gag mutated. The entire pBIVfinalvec was then subjected to DNA sequencing to confirm the integrity of the construct.

### Example 3

### 3.1

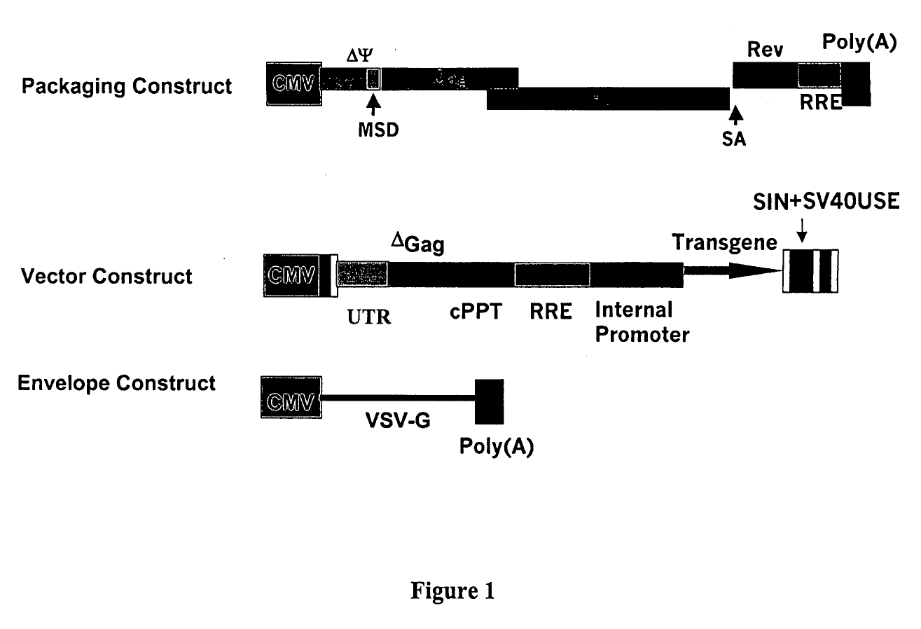

Referring now to FIG. 1, there is shown a schematic representation of the BIV three component gene transfer system containing: (i) the packaging construct, (ii) the transfer vector construct, and (iii) the viral surface protein gene construct. The plasmid construction for the packaging construct pBIVminipack and the transfer vector construct pBIVfinalvec were described in Example 2. The packaging construct, pBIVminipack, contains only the CMV immediate early promoter driving the BIV gag/pol coding sequence, followed by the fused coding sequence for rev, the minimal RRE, and finally an SV40 polyadenylation signal. There still remains the MSD site upstream of the start of gag, and the splice acceptor (SA) site for rev. The transfer vector construct, pBIVfinalvec, has a CMV promoter followed by R, U5, UTR, cPPT, RRE, an internal promoter driving the transgene, modified U3 (SIN), R, and U5. (CMV: CMV early promoter; Δφ: Packaging signal sequence deletion; MSD: Major splice donor site; SA: Splice acceptor site; rev: BIV rev; RRE: BIV Rev response element; UTR: Untranslated leader sequence; ΔGAG: The first 101 bp of BIV gag sequence; cPPT: Central polypurine tract; SIN: Self-inactivating; SV40USE: SV40 polyadenylation signal upstream enhancer element; VSV-G: Vesicular Stomatitis Virus envelope glycoprotein G).

### 3.2

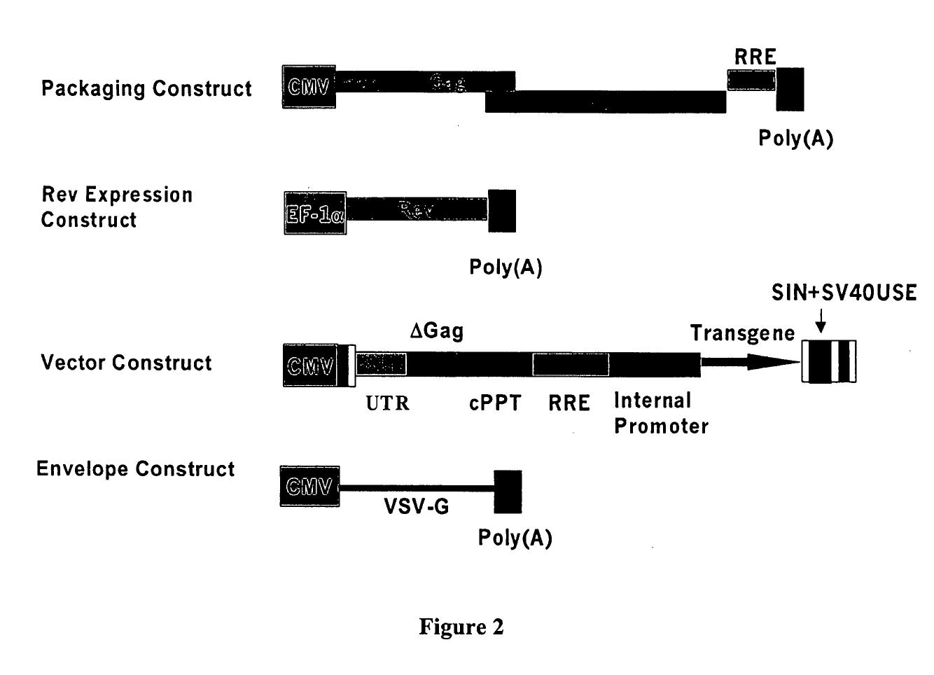

Referring now to FIG. 2, there is shown a schematic representation of the BIV four component gene transfer system which contains (i) the packaging construct without rev, (ii) the rev expression construct, (iii) the transfer vector construct, and (iv) the viral surface protein gene expression construct. The plasmid construction for the packaging construct pCIigpRRE, the rev expression construct pTracerARev, the transfer vector construct pBIVfinalvec, and the viral surface protein gene expression construct were described in Example 2. EF-1á: EF1á promoter.

### Example 4

### Identification of BIV Packaging Signal Sequences

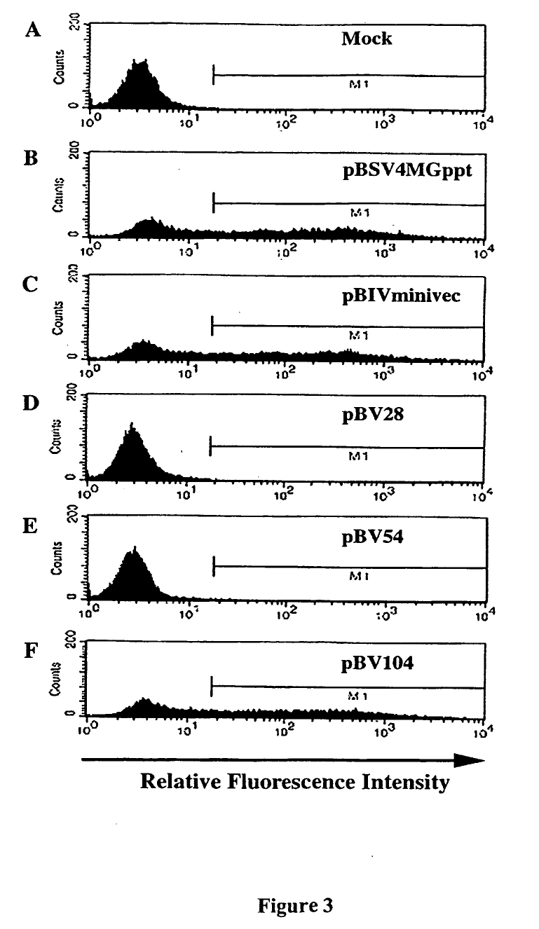

The transfer vector pBIVminivec, with a deletion in gag resulting in only 212 bp remaining in the gag, transduced 293 T cells at the same efficiency as the parental vector pBC4MGppt which had 509 bp of gag sequence. Flow cytometry analysis of eGFP expression in cells transduced with a BIV vector containing either 509 bp of gag sequence or 212 bp gag sequence was performed. eGFP expression was measured by flow cytometry analysis. The following results are given as a percentage of eGFP positive cells: Mock Transduction 0.1%; Cells transduced with vector pBSV4MGppt 85%; Cells transduced with pBIVminivec 95.5%. Further, an additional gag sequence was deleted from the 3′ end in pBIVminivec generating pBIVfinalvec which only contained 28 (pBV28), 54 (pBv54), or 101 (pBv101) bp of gag sequence, respectively. The 101 bp of the gag sequence present in pBv101 and pBIVfinalvecATG are shown in SEQ ID NO:39. The vectors produced from the construct pBv101 were fully functional and were able to transduce cells efficiently while pBv28 and pBv54 were defective (FIG. 3). Therefore, pBv101 containing 101 bp gag sequence was designated as pBIVfinalvecATG. Interestingly, we also found that even for the pBIVfinalvecATG, the RRE is still absolutely required for the vectors' biological function in terms of their ability to transduce target cells as the removal of RRE completely abolished the transduction efficiency (FIG. 3, Panel B). We conclude that the first 101 bp of BIV gag sequence starting from gag ATG contains the BIV packaging signal. This 101 bp of BIV gag sequence, together with the untranslated leader sequence located between 5′ U5 and gag start codon ATG, constitute a minimal BIV packaging signal sequence as delineated in SEQ ID NO:39.

### Example 5

### Identification of BIV Rev Response Element

The plasmid pBC4MGppt contained the putative RRE sequence in a 778 bp envelope coding region (Berkowitz et. al., 2001; PCT patent application WO 01/44458). To determine the precise location of the BIV RRE, various constructs with several independent portions of the putative RRE region were generated. These different constructs were generated to identify the minimal nucleic acid sequence necessary for nuclear export. Determination of the minimal RRE was performed in an effort to reduce the sequence homology between a vector construct having a BIV packaging signal and a BIV gag/pol expression packaging construct. In total, seven different constructs were generated that incorporated seven different regions of the putative RRE (Table 1). All seven constructs were individually transfected into 293 T cells together with the BIV packaging construct pBH2 (Berkowitz et. al., 2001; PCT patent application PCT/US00/33725 (WO 01/44458)) and VSV-G expression plasmid. Forty eight hours post transfection viral supernatant was harvested. Each of these supernatants was normalized by determining the amount of reverse transcriptase (RT) activity (Reverse Transcriptase Assay, Roche Molecular Biochemicals, Indianapolis, Ind., Cat. #1828657) and the same amounts of RT containing vector supernatant was used to transduce the same number of 293 T cells. RT is an accurate measurement of vector particles, the same amounts of RT input represents the same number of vector particle input. Of the seven constructs created, the vector generated from vector construct pBSV4MGpptΔGAGPRERRE6 transduced cells at equivalent efficiency as the parent vector produced from the construct pBSV4MGpptΔGAGPRE with the full length 778 bp putative RRE sequence (Table 1). This data shows that this construct, which contained the 312 bp sequence of SEQ ID NO:40, contains sufficient RRE sequence that is responsible for nuclear export. This 312 bp minimal RRE was used in all our BIV packaging and transfer vector constructs where the RRE is needed.

### Example 6

### 6.1. Cell Lines and Culture Conditions

293 T cells (ATCC; CRL 11268) and HeLa (ATCC; CCL-2) cells were cultured in Dulbecco's Modified Eagle Medium (DMEM; BRL Life Technology, Rockville, Md.) supplemented with 10% heat inactivated fetal bovine serum (Hyclone, Salt Lake City, Utah) 50 IU of penicillin per ml, 50 ug of streptomycin per ml, and 2 mm L-glutamate (Complete DMEM). Mouse neuronal and amoeboid stem cells (Neuro-2A) were obtained from American Type Culture Collection (Manassas, Va.). Neuro-2A cells were cultured in Minimal Essential Medium (BRL Life Technology, Rockville, Md.) supplemented with 10% heat inactivated fetal bovine serum, 50 IU of penicillin per ml, 50 ug streptomycin per ml, 1.0 mm sodium pyruvate, 2 mM L-glutamine, 1.5g/L sodium bicarbonate, and 0.1 mM non-essential amino acids. Human primary skeletal muscle cells (SkMC) (BioWhittaker, Walkersville, Md.) were cultured in SkBM basal media with the SkGM bullet kit containing 0.1% human epidermal growth factor, 1% insulin, 5% BSA, 5% fetuin, 1% gentamicin-amphotericin B, and 0.5 M dexamethasone. Cell lines were maintained at 37° C. in a humidified incubator with 5% CO₂.

### 6.2. Viral Vector Production

293 T cells were seeded at a density of 4×10⁶ into 10 cM dishes overnight. The following day the medium was aspirated and replaced with fresh complete DMEM. The 293 T cells were transfected 4 hours later using the Profection Mammalian Transfection System, calcium phosphate coprecipitation method (Promega, Madison, Wis.). Typically, 15 μg of the transfer vector construct, 15 μg of the packaging construct, and 4.5 μg of the VSV-G viral surface protein gene construct were used to transfect the seeded cells in each dish. In the case of the four construct system, 15 μg of the transfer vector construct, 15 μg of the packaging construct (pCIigpRRE), 9 μg of rev expression construct (pTracerARev), 4.5 μg of the VSV-G viral surface protein gene construct were used to transfect the seeded cells in each dish. After 24 hours, the media was aspirated and replaced with fresh complete DMEM. Viral supernatant was harvested at 48 hours post-transfection, centrifuged at 2000 RPM for 10 minutes to clear cell debris and stored as frozen in aliquots at −80° C. Five microliters of the cleared supernatant was lysed and analyzed for reverse transcriptase(RT) activity using a commercial kit (Reverse Transcriptase Assay, Roche Molecular Biochemicals, Indianapolis, Ind., Cat. #1828657). Generation of VSV-G pseudotyped murine leukemia virus (MLV) vector encoding eGFP was described previously (Berkowitz et. al., 2001).

### 6.3. Transduction

To transduce dividing cells, 2×10⁵ cells were seeded per well into 6-well dishes. After 24 hours, the medium was aspirated and typically 2 mls of viral supernatant (containing approximate 2×10⁶ transducing units of vector particles) were added to the cells. Protamine sulfate was then added to the wells at a final concentration of 8 μg/ml. Cells were then maintained at 37° C. in a humidified incubator with 5% CO₂ for 3 hours. After 3 hours, viral supernatant was aspirated and replaced with fresh medium and incubated for 48 hours at 37° C. in a humidified incubator with 5% CO₂. To transduce non-dividing cells, 1×10⁵ cells were seeded per well into 6-well dishes. After 24 hours, aphidicolin was then added to a final concentration of 4 μg/ml. Sixteen hours post treatment with aphidicolin, the medium was aspirated and typically 2 mls of viral supernatant was added to the cells in the presence of aphidicolin. Protamine sulfate was then added to the wells at a final concentration of 8 μg/ml. Cells were maintained at 37° C. in a humidified incubator with 5% CO₂ for 3 hours. Viral supernatant was then aspirated and replaced with fresh medium containing aphidocolin at a final concentration of 2 μg/ml and the cells were incubated for 48 hours at 37° C. in a humidified incubator with 5% CO₂.

### 6.4. Flow Cytometry Analysis and Titering Method

For analysis of eGFP expression, the medium was aspirated from the wells. The cells were rinsed with 2 mls of phosphate buffered saline (PBS). The PBS was then aspirated and the cells were trypsinized, washed and resuspended in PBS containing 5% heat inactivated fetal bovine serum. The cells were analyzed for eGFP expression on a FACS Calibur (Becton Dickinson Biosciences).

To determine the titer of BIV vectors encoding eGFP, Cf2Th cells in 6-well dishes (4×10⁵ cells/well) were transduced with 2 ml medium containing different dilutions of viral vectors in the presence of protamine sulfate. After 3 hours, viral supernatant was aspirated and replaced with fresh medium and cells were incubated for 48 hours at 37° C. in a humidified incubator with 5% CO₂. Cells were then recovered and assayed by flow cytometry for expression of eGFP. The vector titer was calculated in the following manner: Titer (transducing units/ml)=% positive cells×4×10⁵ cells×dilution factor.

### Example 7

### Gene Expression Mediated by Vectors Generated from BIV Three Construct Based Gene Transfer System

### 7.1. Transduction of Dividing and Non-Dividing Cells

BIV vectors encoding eGFP were generated through cotransfection of 293 T cells with the packaging and transfer vector constructs together with a VSV-G viral surface protein gene construct as described in Example 6.2. The vector supernatants were assayed for reverse transcriptase (RT) activity (Reverse Transcriptase Assay, Roche Molecular Biochemicals, Indianapolis, Ind., Cat. #1828657). Equal amounts of RT-containing nonconcentrated vector supernatants (40 ng RT equivalent vector particles in 2 ml)(except MLV based oncoretroviral vector which was not assayed for RT) were used at equal volume. MLV vector was used as a control to confirm the cell dividing or non-dividing status because MLV based oncoretroviral vectors only transduce dividing cells but can not transduce non-dividing cells. The MLV vector was then used to transduce both dividing and non-dividing (see Example 6.3) HeLa and Neuro-2A cells (Table 2). While VSV-G pseudotyped MLV efficiently transduced both dividing HeLa and Neuro-2A cells, aphidicolin treated cells (aphidicolin was used at a final concentration of 1 μg/ml)were fully resistant to the MLV mediated transduction as expected (Table 2) indicating the aphidicolin treated cells were probably not dividing under our experimental conditions. However, both non-dividing HeLa and Neuro-2A were transduced with the nonconcentrated minimized BIV vector at relatively high efficiency indicating that the vectors generated from the minimized BIV packaging and minimized transfer vector constructs are fully competent to mediate transgene expression in both dividing and non-dividing cells.

TABLE 2

Percentage of eGFP Positive Cells

| | | |
|---|---|---|
| Vector | Dividing | Nondividing |

HeLa Cells

| | | |
|---|---|---|
| Mock infected | 0.1% | 0.01% |
| MLV viral vector | 51.8% (SD = 1.99) | 0.44% (SD = 0.11) |
| BIV viral vector | 36.1% (SD = 0.47) | 39.5% (SD = 4.3) |

Neuro-2A Cells

| | | |
|---|---|---|
| Mock infected | 0.12% | 0.12% |
| MLV viral vector | 44.6% (SD = 0.72) | 0.83% (SD = 0.07) |
| BIV viral vector | 57.2% (SD = 0.56) | 30.4% (SD = 0.15) |

Human Primary Skeletal Muscle Cells

| | | |
|---|---|---|
| Mock infected | 0.03% | 0.05% |
| MLV viral vector | 25.6% (SD = 1.5) | 0.23% (SD = 0.5) |
| BIV viral vector | 55.1% (SD = 5.8) | 69.4% (SD = 3.2) |

### 7.2. Transduction of Human Primary Cells

To examine the ability of the minimized BIV vector to transduce and express in primary cells, human primary skeletal muscle cells (BioWhittaker, Walkersville, Md.) were treated with alphidicolin (aphidicolin was used at a final concentration of 1 μg/ml) or without alphidicolin. The MLV based vector transduced the dividing primary cells very well but did not score any significant transduction in non-dividing cells (Table 2). On the contrary, the nonconcentrated minimized BIV vector efficiently transduced both the dividing and non-dividing primary cells (Table 2) confirming that the significant minimization of the BIV packaging and transfer vector constructs did not affect the ability of vector to transduce the human primary non-dividing cells.

### Example 8

### Comparison of Vectors Generated from BIV Three Component and Four Component Based Gene Transfer Systems

Table 3 shows a comparison of flow cytometry analysis of eGFP expression in vectors generated from BIV three construct and four construct based gene transfer systems in transduced 293 T cells. 293 T cells were transduced with either mock (Mock herein means the cell culture medium harvested from the 293 T cells in the absence of vector transfection), vectors produced from the BIV three component system, or vectors generated from the BIV four component system (Table 3) as described in Example 6.2. Equal amounts of vector particles (40 ng RT equivalent vector particles) were used for the BIV three and four component systems as measured by RT activities. The results (Table 3) demonstrate that the BIV four component based gene transfer system produced vectors at equal quality as the vectors generated from the three component system. Moreover, the four component system is BIV REV dependent as no functional vectors were generated in the absence of the BIV rev expression construct pTracerARev (Table 3: Minus Rev).

TABLE 3

| | Percent eGFP |
|---|---|
| | Positive Cells |
| Mock Infected | 0% |
| BIV Three Component System | 15.26% |
| BIV Four Component System Minus Rev | 0% |
| BIV Four Component System Plus Rev | 14.92% |

### Example 9

### Replacement of the BIV Central Polypurine Tract (BIVcPPT) by HIV Central Polypurine Tract (HIVcPPT)

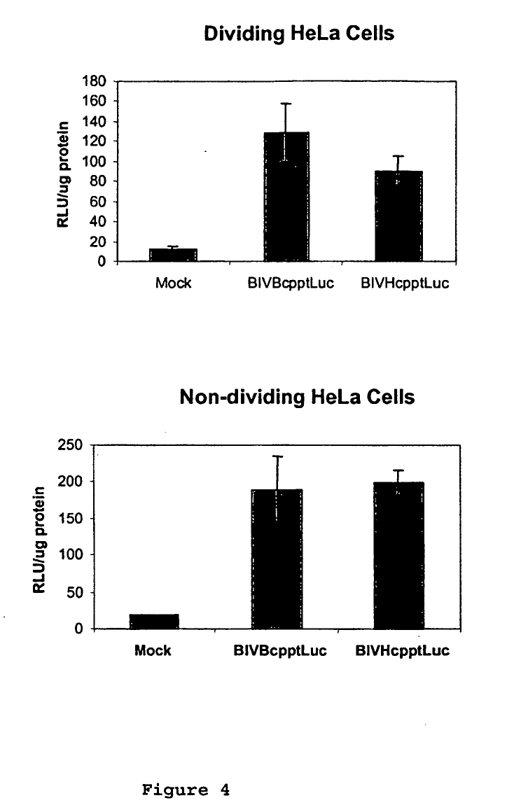

In order to minimize the overlaps between the packaging and transfer vector constructs, the cPPT in the BIV transfer vector construct was replaced with HIVcPPT. Specifically, pBIVminivec was digested with ClaI and EcoRI to remove BIVcPPT, blunt ended and ligated with a HIVcPPT as a blunt end ligation. The HIVcPPT was described by Charneau et al (Charneau et al., 1992. J. Virology, 65:2415-2421). The vectors were generated and the vector particles containing a different cPPT were normalized by RT. The same amounts of RT were used to transduce both dividing and non-dividing HeLa cells. The results suggested that the vectors with either BIVcPPT or HIVcPPT transduced both dividing and non-dividing target cells equally well, indicating that BIVcPPT can be replaced by HIVcPPT functionally (FIG. 4). The minimal cPPT of BIV consists essentially of 535 base pairs corresponding to the nucleotides from base pairs 4374 to 4909 in the pol coding region of BIV RNA genomic sequence (BIV isolate 127).

### Example 10

### Identification of Ribosomal Frameshifting Site in BIV GAG/POL Expression

For some retroviruses and lentiviruses, the gag and pol genes lie in different translational reading frames, with the 3′ end of gag overlapping the 5′ end of pol. Therefore, production of GAG-POL fusion protein would require either messenger RNA processing or translational frameshifting. The latter mechanism has been shown in the synthesis of the GAG-POL proteins of Rous sarcoma virus (RSV), avian sarcoma/leukosis virus (ASLV), mouse mammary tumor virus (MMTV), human immunodeficiency virus (HIV), simian immunodeficiency virus (SIV), and feline immunodeficiency virus (FIV). Studies on these viruses have shown that ribosomal frameshifting requires a seven-base sequence at the FRAME SHIFT site and a secondary structure element (i.e., stem-loop) immediately downstream of the site. Examples of shifty (the term “shifty” as used herein means the ribosomal translational complex moves backward one nucleotide and starts translating another protein using the same mRNA) sequences are A AAU UUA (SEQ ID NO:41; ASLV), U UUU UUA (SEQ ID NO:42; HIV-1), A AAA AAC (SEQ ID NO:43; MMTV) and G GGA AAC (SEQ ID NO:44; FIV). Thus, the general form of the shift site is the sequences X XXY YYZ, in which the triplets are the initial (or “0”) translation frame and X may be identical to Y.

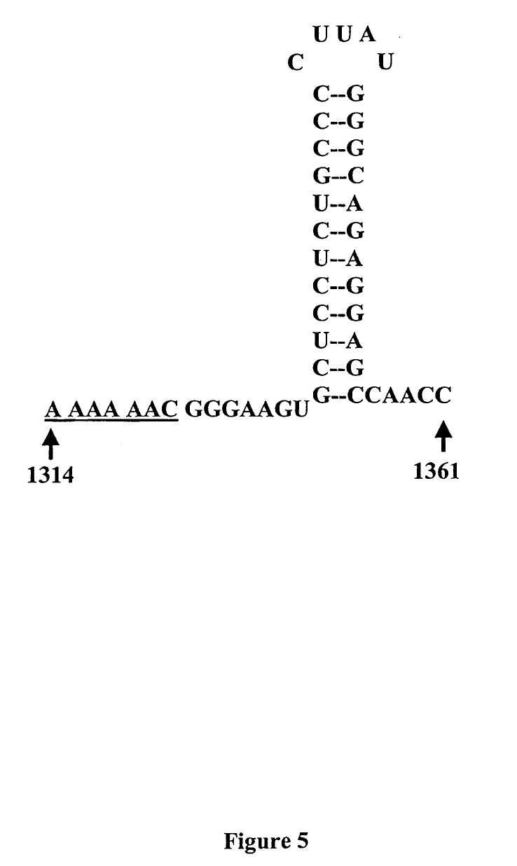

Unlike HIV or SIV, BIV has not been studied extensively. The precise location of translational frameshifting for the BIV pol gene translational start site has not been determined previously. It was proposed that the frameshifting occurs at the sequence C AAA AAT (SEQ ID NO:45, where C is at the nucleotide 1576 in the BIV viral genomic RNA strain 127 (Garvey et al, Virology, 175:391-409, 1990). However, we identified that the translational frameshifting instead takes place in the sequences A AAA AAC (SEQ ID NO: 46), corresponding to nucleotides 1629 to 1635 in the BIV viral genomic RNA and corresponding to nucleotides 2013 to 2019 of SEQ ID NO:1, the BIV provirus DNA sequence. Furthermore, we have discovered a stem-loop immediately following the frameshifting sequences as indicated by the drawing in FIG. 5. The viral sequence and structure, indicated in FIG. 5 and as shown also in SEQ ID NO:47 (DNA) and SEQ ID NO:48 (RNA), constitute the Bovine Immunodeficiency Virus Gag/Pol ribosomal frameshifting site.

### Example 11

### A BIV Packaging Construct with Recoded gag/pol Sequences

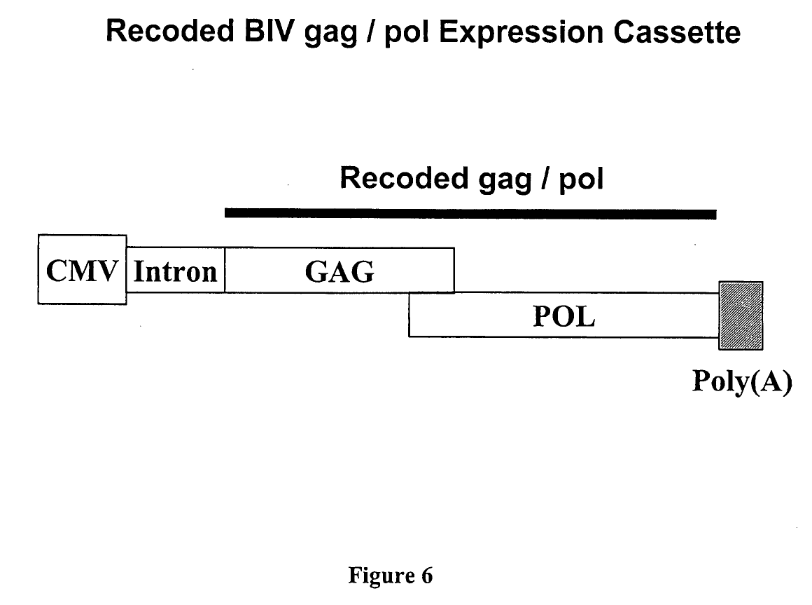

Lentiviruses such as HIV, SIV and BIV are thought to contain nucleic acid sequences in their viral RNAs which cause RNA instability, thereby preventing efficient nuclear export of viral RNAs. This is believed to be due to the fact that lentiviruses employ rare codon usage and/or RNA secondary structure which is determined by the RNA sequence. The viral RNAs containing these rare codons cannot be efficiently transported out of the nucleus without rev/RRE. To eliminate RRE from the packaging construct, to minimize or eliminate the overlaps between the packaging and transfer vector constructs and to increase the BIV gag/pol gene expression levels, we recoded the BIV gag/pol coding sequence using preferred Homo sapiens codons (SEQ ID NO:49). The recoded gag/pol coding sequence was cloned into the pCI mammalian expression vector, generating pCIigpSyn (FIG. 6). The synthetic BIV gag/pol gene was constructed using techniques known in the art and those described in PCT publication WO 01/68835. Specifically, a Xhol site and a XbaI site were incorporated into the flanking 5′ and 3′ ends of the recoded gag/pol coding region respectively when the recoded gag/pol was synthesized. The recoded gag/pol was then digested with XhoI and XbaI and cloned into pCI expression construct (Promega, Madison, Wis.) which was digested with XhoI and XbaI previously, creating pCIgpSyn. The generation of pCIigpSyn allowed us to produce BIV vectors from the four component system by cotransfecting pCIigpSyn, pTracerARev, pBIVfinalvec, and pCMVVSV-G. The BIV vectors generated from this system with recoded gag/pol were fully functional as indicated by their ability to efficiently transduce cells. Table 4 shows results from flow cytometry analysis of eGFP expression in 293 T cells. 293 T cells were transduced by BIV vectors generated from the four component system, except in the case of “Rev Minus” where viral vector production was performed in the absence of the Rev expression component. This experiment compared viral vectors produced from the wild-type BIV gag/pol expression component to the viral vector produced from the recoded BIV gag/pol expression component (Table 4).

TABLE 4

| | Percent eGFP |
|---|---|
| | Positive Cells |
| Mock Transduced | 0.02% |
| Wild-type BIV gag/pol | 85% |
| Recoded BIV gag/pol Minus Rev | 0.9% |
| Recoded BIV gag/pol Plus Rev | 50% |

Recoding of a gene or portions of a gene can be performed using techniques well known in the art. By way of non-limiting examples, Casimiro D R et al. describes a PCR-based method for gene synthesis(Structure Nov. 15, 1997;5(11):1407-12) (See also Brocca et al. “Design, total synthesis, and functional overexpression of the Candida rugosa lip 1 gene coding for a major industrial lipase” Protein Sci June 1998;7(6):1415-22; Withers-Martinez C, et al., “PCR-based gene synthesis as an efficient approach for expression of the A+T-rich malaria genome” Protein Eng December 1999;12(12):1113-20; and Stemmer et al., “Single-step assembly of a gene and entire plasmid from large numbers of oligodeoxyribonucleotides” Gene Oct. 16, 1995;164(1):49-53).)

To compare the packaging construct with recoded gag/pol to the packaging construct with the wild type gag/pol, the ability of the packaging construct to complement vector production was tested. BIV vectors were produced as described in Example 6.2. The BIV packaging construct with the recoded gag/pol is more potent than the packaging construct with wild type BIV gag/pol. BIV vector was produced either with pCIigpRRE or with pCIigpSyn at 10-fold lower plasmid input (1.5 μg vs 15 μg). The vector produced by the recoded gag/pol transduced a higher percentage of cells (47%) than those transduced with vector produced by the packaging construct containing wild type gag/pol (27%), as indicated by eGFP expression analyzed by FACS. Mock transduced cells transduced 0% of the cells. Equal volumes of vectors were used for both the recoded gag/pol and wild type gag/pol samples. The data demonstrate that the packaging construct pCIigpSyn with recoded gag/pol is more potent than the packaging construct pCIigpRRE with the wild type gag/pol. Also, the recoded gag/pol was fully functional in the absence of the RRE element. The absence of the RRE in the recoded gag/pol construct served to further minimize or eliminate homology with the transfer vector, which contains a wild-type RRE.

Having determined the ribosomal frameshifting site as in Example 10, the amino acid sequence of the BIV pol gene was determined using standard DNA codon and open reading frame analyses. The nucleotide sequence of the wildtype BIV pol gene is shown in SEQ ID NO:50. The deduced amino acid sequence of the BIV pol gene, which is based on the identification of the ribosomal frame shifting site between the gag and pol genes, is shown in SEQ ID NO:51. Since the amino acid sequence of BIV pol was determined, this facilitated recoding of the BIV pol gene. Having determined the amino acid sequence of the BIV pol gene, a person of ordinary skill in the art could modify this sequence to be recoded in order to optimize expression in a particular cell type. Using methods similar to those employed for recoding the gag/pol combination DNA fragment, the DNA of the pol gene was also recoded. The recoded BIV DNA pol gene is shown in SEQ ID NO:52.

As noted in SEQ ID NO: 51, the BIV pol gene does not code for an initial met amino acid and as noted in SEQ ID NO:52, there is not contained at the 5′ end of this gene a codon for met and initiation of protein synthesis. In order to construct BIV transfer systems wherein the gag and pol genes are provided on separate constructs, a synthetic DNA is constructed which encodes the pol gene having an additional codon at the 5′ end of the open reading frame which enables synthesis of a POL polypeptide having an initial met codon. The sequence of this synthetic DNA is shown in SEQ ID NO:53. The sequence of a synthetic DNA that has been recoded for pol and also contains an met start codon is shown in SEQ ID NO: 54.

### Example 12

### An Inhibitor of Histone Deacetylase Increases Lentiviral Vector Production and Enhances Vector Transduction Efficiency

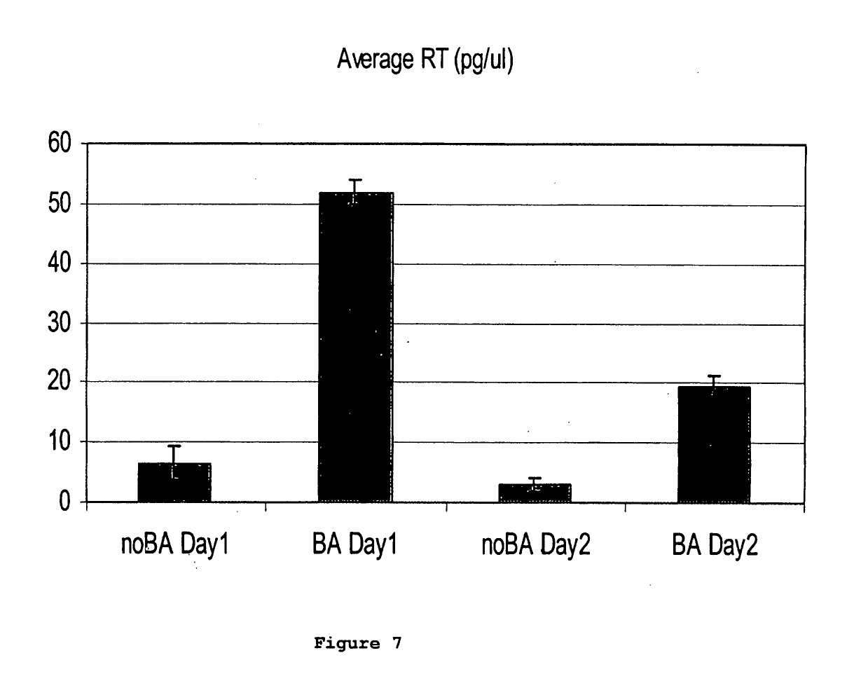

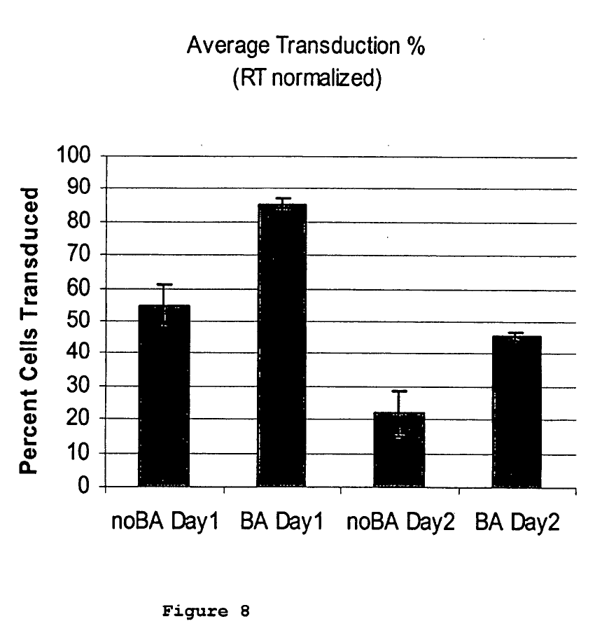

One of the technical hurdles associated with broad application for lentiviral vectors is the relatively low titer (approximately 10⁵ to 10⁷ transducing units/ml depending on various vector systems). To overcome this hurdle, the vectors can be concentrated through ultracentrifugation. It is important to increase the titer of a lentiviral vector through means that will not affect the quality of the vectors. In one embodiment of the invention, a histone deacetylase inhibitor, Butyric Acid (BA), significantly increases the production of Bovine Immunodeficiency Virus based lentiviral vectors by 5 to 10 fold, as indicated by the reverse transcriptase activity found in the medium of the vector producing cells (FIG. 7). Specifically, Butyric Acid (Sigma, St. Louis, Mo.) was added to the vector producing cells at a final concentration of 5 mM 24 hours before harvesting the vector. Reverse transcriptase activity is a measurement of viral vector particles. Furthermore, the vectors produced in the presence of butyric acid are more infectious as measured by transduction efficiency (FIG. 8). The BIV vector particles were produced as described in Example 6.2.

### Example 13

### In vivo Transduction of Retinal Pigment Epithelial Cells with BIV Vectors

Retinal pigment epithelial cells (RPE) are one of the targets in the eye for ocular gene therapy. To test the transduction efficiency of RPE with BIV vectors, BIV vectors encoding eGFP were generated from the three component system and injected into mouse eye via subretinal injection (5×10⁵ transducing units/per eye). The eye tissue was harvested, sectioned, and examined for eGFP expression at different time points ranging from one week to ten weeks after injection. The sectioned tissue was directly examined by immunofluorescence microscopy for eGFP expression or detected with immunohistochemistry staining. A significant portion of retinal pigment epithelial (RPE) cells was transduced by BIV vectors as indicated by eGFP expression. Eyes were cryosectioned at 1, 2, 8 and 10 weeks after vector injection. At one week GFP expression is seen in the RPE layer. At two weeks after vector injection eGFP expession was seen in RPE layer and was also detected in the retina, photoreceptor and glial cells. At 8 weeks after vector injection, eGFP expression was seen in both the RPE layer and retina. At 10 weeks after vector injection, eGFP expressing RPE cells were clearly seen in the fundus.

### Example 14

### BIV Vector Mediated Transgene Expression in Mouse Brain

Lentiviral vector mediated gene expression has great potential for a variety of applications for treatment of human diseases. Neuronal and ocular diseases represent two promising areas that are most suitable for lentiviral vector based gene therapy. Recombinant BIV based lentiviral vectors of the invention were tested for the ability to mediate transgene expression in mouse brain. BIV vector encoding eGFP (1×10⁶ transducing units in 2 μl) was injected into mouse substantia nigra. Seventeen days after injection, section of the mouse brain was examined for eGFP expression by immunohistochemistry staining. Cells in mouse brain were transduced at a relatively high efficiency. Some of the cells were indeed neuronal cells as indicated by co-staining of eGFP (green) and NeuN (red, neuronal cells specific marker) resulting in yellow spots.

### Example 15

### BIV Vector Mediated Transgene Expression in Rat Spleen Following Systemic Delivery

A BIV vector encoding luciferase vector (1×10⁹ transducing units in 250 □l) was injected via i.v. (tail vein) into rats. As control, TBS (Tris-buffered Saline) was also injected into a control group of rats as negative control. Fifteen days after vector injection, the rats were administered with luciferin (a substrate of luciferase) and the rats were examined with the Xenogen Image System. The rat spleen was efficiently transduced by the BIV vector and high levels of luciferase expression were observed through the image system. No significant signal could be detected under the same conditions in the negative control rats.

### Example 16

### Inhibition of Ocular Neovascularization in vivo by BIV Vector Mediated Anti-Angiogenesis Gene Expression

Many neovascularization related ocular diseases such as age-related macular degeneration (AMD), for example, have no effective therapy and represent major unmet medical needs. As shown in Example 13, recombinant BIV-based vectors of the present invention efficiently transduced mouse retinal pigment epithelial cells. A BIV vector encoding murine endostatin, an anti-angiogenesis gene (O'Reilly et al., Cell;88(2):277-85 (1997)), was administered via subretinal injection of transgenic mice (IRBP/rtTA-TRE/VEGF tgMICE) that express Vascular Endothelial Growth Factor from mouse photoreceptor cells upon induction with Doxycyclin. BIV vectors were injected into mouse right eyes while the left eyes served as control without injection of vectors. Three weeks after vector injection, 0.5 mg/ml of Doxycyclin was placed in the drinking water for the transgenic mice. Five days after introduction of Doxycyclin, results were analyzed. Doxycyclin induced VEGF expression resulting in severe neovascularization on the left eyes of the transgenic mice as shown by the fluorescein angiograms. The VEGF induced neovascularization was completely blocked by BIV vector mediated endostatin expression in the right eyes in the same animals.

### Example 17

### HIV and BIV do not Cross Package

One of the issues associated with lentiviral vector based gene therapy is safety. One could imagine that if a patient is infected with HIV, the wild type HIV could potentially serve a packaging function to generate new recombinants by mobilizing lentiviral vectors intended for therapeutic use. To address this question, cross packaging experiments were performed in which a BIV based packaging construct was employed to produce a BIV based vector (BIV/BIV) or an HIV based packaging construct was employed to generate an HIV based vector (HIV/HIV). For testing of cross packaging, we used a BIV based packaging construct to try and produce an HIV based vector packaged into vectors (BIV/HIV), or an HIV based packaging construct to try and produce a BIV based vector (HIV/BIV) packaged into vectors. The vectors were generated and equal amounts of vector particles as indicated by RT activity assay were used to transduce equal number of Cf2Th cells. Analysis of eGFP expression in Cf2Th cells transduced with HIV vectors, BIV vectors, HIV/BIV cross packaged vectors or BIV/HIV cross packaged vectors was analyzed by FACS. Both BIV vector generated from BIV/BIV and HIV vector generated from HIV/HIV transduced 31% and 21% of the cells, respectively. However, vector particles produced by either BIV/HIV or HIV/BIV pairs did not yield detectable eGFP positive cells indicating the vectors produced by these two pairs were empty or defective particles. Mock transduction was used as a negative control with 0% of the cells being eGFP positive. No difference between seen between mock, BIV/HIV or HIV/BIV infected cells. Our data indicate that HIV packaging construct cannot cross package BIV vector production and BIV packaging construct cannot cross package HIV vector. In a further analysis, the HIV packaging construct was co-transfected with the BIV vector construct and the BIV Rev construct to ensure nuclear export of the BIV vector RNA. Again no transduction of target cells was observed further indicating that HIV can not package, or at least not efficiently, BIV vectors.

### Example 18

### Mutation of BIV Protease

One of the major hurdles encountered when producing a stable lentiviral based packaging cell line is the inability to maintain high levels of expression of GAG and POL proteins. The catalytic center of HIV protease includes a three amino acid motif, Asp-Thr-Gly (Konvalinka, J. et al., J. Virol. 69:7180-7186, 1995) These three amino acids are conserved among HIV and SIV isolates documented so far (Korber B, Theiler J, Wolinsky S, Science Jun. 19, 1998 280: 5371 1868-71). Konvalinka, J. et al. mutated the Thr residue (corresponding to amino acid number 26 from the start of protease in HIV isolate HXB2) to a Ser. They found that the mutated HIV protease has a significantly reduced toxicity while preserving the protease activity. This information makes it possible to generate a stable cell line to express high levels of lentiviral Gag/Pol proteins. Expression of these proteins is absolutely necessary in order to establish a stable packaging cell line for lentiviral vectors, in particular for HIV- or BIV-based lentiviral vectors. The Asp-Thr-Gly motif is also present in BIV protease in the same location. A comparison of the first 29 Amino Acids of HIV and BIV proteases reveals that the amino acids number 25 to 29 are identical between HIV and BIV proteases, including the said Asp-Thr-Gly motif:

| | |
|---|---|
| HIV Protease (HXB2): | |
| 1-PQVTLWQRPLVTIKIGGQLKEALLDTGAD | (SEQ ID NO:55) |
| BIV Protease (127 isolate): | |
| 1-SYIRLDKQPFIKVFIGGRWVKGLVDTGAD | (SEQ ID NO:56) |
| HIV Protease mutation: | |
| 1-PQVTLWQRPLVTIKIGGQLKEALLDSGAD | (SEQ ID NO:57) |
| Corresponding BIV Protease mutation: | |
| 1-SYIRLDKQPFIKVFIGGRWVKGLVDSGAD | (SEQ ID NO:58) |

A point mutation was made in the packaging construct pCIigpSyn at the amino acid Thr of SEQ ID NO:40 wherein the Thr at amino acid 26(coded by nucleotides ACT corresponding to nucleotides from 1806 to 1808 in BIV viral genomic RNA isolate 127, Garvey et al., 1990; SEQ ID NO: 56 and SEQ ID NO:58 represent partial sequences of the BIV protease, the full sequence of which is encoded in one embodiment of the vectors of the present invention, either in mutated or recoded form) was replaced with Ser at the same position without any change in any other coding region of the packaging construct. This packaging construct with a Thr to Ser mutation was designated as pCIigpSynSer. pCIigpSynSer was compared to pCIigpSyn for the ability to support BIV vector production and the transduction efficiency achieved by the BIV vectors.

Specifically, 8×10⁶ 293 T cells in 10-CM dishes were transfected with pCligpSyn or pCIigpSynSer (1 ug), pTracerARev (10 ug), pBIVminivec (15 ug), and pCMVVSV-G (4.5 ug). Forty-eight hours after transfection, vectors were harvested from the transfected cells. HeLa cells were transduced with equal numbers of vector particles as indicated by reverse transcriptase (RT) activity. Forty-eight hours after transduction, flow cytometry analysis was performed to score eGFP positive HeLa cells. As indicated in Table 5, the vector generated by the packaging construct with the Thr to Ser mutation pCIigpSynSer, transduced HeLa cells as efficiently as the vector produced by the packaging construct pCIigpSyn. The nucleotide sequence for this mutated gag/pol gene is shown in SEQ ID NO:59.

TABLE 5

| Packaging Construct | Transduction Efficiency | Mean GFP Intensity |
|---|---|---|
| Mock | 0% | 0 |
| pCIigpSyn | 91% | 1000 |
| pCIigpSynSer | 92% | 1050 |
| Comparison of BIV vector mediated eGFP expression in HeLa cells. BIV vectors encoding GFP were generated either by the packaging construct, pCIigpSyn or pCIigpSynSer and were compared for their transduction efficiencies of HeLa cells and intensity of eGFP expression. Transduction efficiency was measured by the percentage of the positive HeLa cells. Mean | | |
| # eGFP intensity was scored by relative fluorescence intensity. Both transduction efficiency and mean eGFP intensity were analyzed by flow cytometry analysis on a FACS Calibur (Becton Dickinson Biosciences). | | |

### Example 19

### Removal of cPPT from BIV Transfer Vectors

One of the BIV transfer vector constructs has a putative cPPT (Berkowitz et al., 2001b; Matukonis et al., 2002; Molina et al., 2002). To determine if the BIV cPPT was required for transduction efficiency in vitro and in vivo, we compared vectors with or without cPPT in non-dividing cultured HeLa cells and in rat retina. A version of the vector without the putative cPPT was generated by digestion of pBIVminivec with ClaI and EcorI to remove the cPPT and the fragment was blunt ended and religated.

The vectors with or without cPPT transduced 35% and 34% of non-dividing HeLa cells respectively with equal vector particle input. Furthermore, removal of the cPPT did not significantly affect gene transfer efficiency in vivo for the following experiment. BIV vectors with or without cPPT encoding eGFP were injected into rat subretinal space (4.8×10⁵ T.U./per eye) of the right eyes with the left eyes served as controls with 5 rats per group. Two weeks post-injection, the retinal flat mount was examined for eGFP expression directly under a fluorescence microscope.

Results: The left eyes that were not injected did not display any detectable GFP expression. Whereas, the right eyes that were injected with eGFP BIV-vectors displayed substantial amounts of GFP expression. Both the cPPT containing and cPPT deleted BIV-vectors transduced similar amounts of cells in the eye (data not shown).

This demonstrates that in vitro and in vivo transduction can be achieved using a BIV vector that does not contain the putative cPPT. Also, removing the cPPT eliminates a 364 bp block of sequence similarity with the packaging construct. These modifications resulted in a system in which the transfer vector and packaging constructs share only 101 bp of sequence (packaging signal) similarity with no identity longer than 8 bps. Recoding of the gag/pol as described in Example 11 will remove the 101 bp block of homology.

### Example 20

Treatment of POAG may be accomplished by delivering to a patient a vector of the invention encoding the Optineurin gene (Rezaie et al., Science 295: 1077-1079 (2002)) and/or the trabecular meshwork protein gene (TIGR; Stone et al., Science 275:668-670). The vector would preferably be delivered by direct intraocular injection in to the eye. Methods of injection into the eye are well known in the art.

Diseases caused by the degeneration of photoreceptors include, but are not limited to, inherited retinal dystrophies (e.g., retinitis pigmentosa, age-related macular degeneration, Bardet-Biedel syndrome, Bassen-kornzweig syndrome, best disease, choroidema, gyrate atrophy, congenital amourosis, refsun syndrome, stargardt disease and Usher syndrome), retinal detachment, age-related macular degeneration and other maculopathies. Treatment of these diseases may be accomplished by delivering to a patient a vector of the invention encoding for delivering to a patient a vector of the invention encoding a Rod-derived Cone Viability Factor (RdCVF; PCT Application PCT/EP02/03810 (WO 02/081513)) or anti-apoptotic genes.

In addition, the vectors of the invention are useful for expressing optineurin, TIGR, antiogenesis genes and the like in order to treat diseases such as, e.g., choroidal neovascularization due to histoplasmosis and pathological myopia as well as choroidal neovascularization that results from angioid streaks, anterior ischemic optic neuropathy, bacterial endocarditis, Best's disease, birdshot retinochoroidopathy, choroidal hemangioma, choroidal nevi, choroidal nonperfusion, choroidal osteomas, choroidal rupture, choroideremia, chronic retinal detachment, coloboma of the retina, Drusen, endogenous Candida endophthalmitis, extrapapillary hamartomas of the retinal pigmented epithelium, fundus flavimaculatus, idiopathic, macular hole, malignant melanoma, membranproliferative glomerulonephritis (type II), metallic intraocular foreign body, morning glory disc syndrome, multiple evanescent white-dot syndrome (MEWDS), neovascularization at ora serrata, operating microscope burn, optic nerve head pits, photocoagulation, punctate inner choroidopathy, rubella, sarcoidosis, serpiginous or geographic choroiditis, subretinal fluid drainage, tilted disc syndrome, Taxoplasma retinochoroiditis, tuberculosis, or Vogt-Koyanagi-Harada syndrome, among others.

### Example 21

### Therapeutic Transfer and Expression of RdCVF Genes with BIV Vectors

Photoreceptors are a specialized subset of retinal neurons that are responsible for vision. Photoreceptors consist of rods and cones, which are the photosensitive cells of the retina. Each rod and cone elaborates a specialized cilium, referred to as an outer segment, that houses the phototransduction machinery. The rods contain a specific light-absorbing visual pigment, rhodopsin. There are three classes of cones in humans, characterized by the expression of distinct visual pigments: the blue cone, green cone and red cone pigments. Each type of visual pigment protein is tuned to absorb light maximally at different wavelengths. The rod rhodopsin mediates scotopic vision (in dim light), whereas the cone pigments are responsible for photopic vision (in bright light). The red, blue and green pigments also form the basis of color vision in humans. The visual pigments in rods and cones respond to light and generate an action potential in the output cells, the rod bipolar neurons, which is then relayed by the retinal ganglion neurons to produce a visual stimulus in the visual cortex.

In humans, a number of diseases of the retina involve the progressive degeneration and eventual death of photoreceptors, leading inexorably to blindness. Degeneration of photoreceptors, such as by inherited retinal dystrophies (e.g., retinitis pigmentosa), age-related macular degeneration, glaucoma and other maculopathies, or retinal detachment, are all characterized by the progressive atrophy and loss of function of photoreceptor outer segments. In addition, death of photoreceptors or loss of photoreceptor function results in partial deafferentation of second order retinal neurons (rod bipolar cells and horizontal cells) in patients with retinal dystrophies, thereby decreasing the overall efficiency of the propagation of the electrical signal generated by photoreceptors. Secondary glial and pigment epithelium changes secondary to photoreceptors degeneration result in vascular changes leading to ischemia and gliosis. Trophic factors that are capable of rescuing photoreceptors from cell death and/or restoring the function of dysfunctional (atrophic or dystrophic) photoreceptors may represent useful therapies for the treatment of such conditions. In addition, BIV transfer vectors expressing the RdCVF genes can be regulatably expressed in cells in order to determine the physiologic effect of over expressing or underexpressing these genes and the relationship of expression to various diseases of the eye.

The progression of these conditions points to a sequential loss of the two classes of photoreceptors: initially rods are lost as a direct result of a genetic or environmental or unknown lesion, resulting in night blindness and a reduction in visual field followed inevitably by loss of cones leading to total blindness. Thus, cones die indirectly since they do not express the primary lesion.

Rod-derived Cone Viability Factor (RdCVF) has been found to be a cone protective factor (PCT Application PCT/EP02/03810 (WO 02/081513)). Rod-derived Cone Viability Factors (RdCVFs) are expressed in eye tissue and in particular are produced in rod cells. The RdCVF gene may be expressed by other cell types in the local area of the rod cells and still provide a protective benefit. In a human afflicted with a retinal dystrophy the production of RdCVF decreases in amounts relative to expression in the corresponding tissues of humans who do not suffer from retinal dystrophy. Messenger RNA transcribed from the RdCVF genes, and protein translated from such mRNA, is present in rod tissues and/or associated with such tissues in an amount at least about half, preferably at least about five times, more preferably at least amount ten times, most preferably at least about 100 times less than the levels of mRNA and protein found in corresponding tissues found in humans who do not suffer from a retinal dystrophy. Such decreases in transcription of RdCVF mRNA is referred to herein as “decreased transcription.”

In a preferred embodiment, the BIV vector contains a nucleic acid sequence encoding for a RdCVF. SEQ ID NO:60 and SEQ ID NO:62 are the murine RdCVF 1 and RdCVF2 genes respectively and SEQ ID NO:61 and SEQ ID NO:63. encode murine RdCVF1 and RdCVF2 polypeptides respectively (Genbank Accession numbers XM_{—}134263, BC017153 and BC02191 1). SEQ ID NO:64 and SEQ ID NO:66 are the genes for human RdCVF1 and human RdCVF2, respectively and are further described in Genbank Accession numbers NM_{—}138454 and BC014127. Amino acid sequences for the human RdCVF1 and RdCVF2 polypeptides are shown in SEQ ID NO:65 and SEQ ID NO:67 respectively.

A person of ordinary skill in the art will recognize that a nucleic acid encoding a RdCVF can be modified by changing codons without changing the amino acid sequence of the final RdCVF protein. Also provided is a variant of the nucleic acid encoding a RdCVF, wherein the variant encodes a corresponding functional variant of the amino acid sequence of a RdCVF protein. A functional variant may differ in amino acid sequence by one or more substitutions, additions, deletions or truncations which may be present in any combination, but would retain the same biological function as the reference RdCVF.

“Biological function” within the meaning of this application is to be understood in a broad sense. It includes, but is not limited to, the particular functions of the RdCVF protein disclosed in this application. Thus, biological functions are not only those which a polypeptide displays in its physiological context, i.e. as part of a living organism or cell, but includes functions which it may perform in a non-physiological setting, e.g. in vitro. For example, a biological function of the RdCVF protein within the meaning of this application is the ability, for example, to demonstrate a cone protective effect either in vitro or in vivo. Assays to assess the required properties are well-known to a person of ordinary skill in the art.

Also, minor substitutions, deletions and insertions of codons can be made that do not eliminate the therapeutic effect of a RdCVF protein. Thus, while SEQ ID NO's 61, 63, 65, and 67 encode for RdCVF proteins, the invention includes a BIV vector encoding any RdCVF protein having the same or similar therapeutic effect or biological function. In a preferred embodiment, the BIV vector encodes for a human RdCVF1 and/or RdCVF2.

A BIV vector expressing RdCVF may be used to treat various diseases related to the eye. Such a vector can also be used to analyze the physiological effects of RdCVF expression in cells. The RdCVF gene or genes are inserted into the BIV vectors of the invention and the vectors are transferred to eye cells, where the RdCVF genes are expressed at levels equivalent to expression in normal eye cells.

Vectors containing heterologous genes of interest as described in the present application will have various uses including, but by no means limited to, expression of the heterologous gene of interest for gene therapy. As will readily be appreciated by a person of ordinary skill in the art, these vectors can be used to modify gene expression in order to determine the effect of such modification on cell growth, viability and function. For example, modification of gene expression can provide insight into various physiological phenomena such as, for example U.S. Pat. No. 6,465,715 (development in C. elegans); U.S. Pat. No. 6,465,246 (tumorgenesis); and U.S. Pat. NO. 6,461,807 (drug screening by modification of drug target).

### Example 22

### Thogoto Virus Envelope Glycoprotein Pseudotyped Lentiviral Vector

VSV-G has been widely used as an envelope glycoprotein to pseudotype various lentiviral vectors. However, VSV-G is toxic to cells. Thogoto virus envelope glycoprotein is tested for its ability to pseudotype BIV vectors. Thogoto virus glycoprotein coding sequence (SEQ ID NO:68) is cloned into pCI expression plasmid at XhoI site as a blunt-end ligation. The resulting construct, pCI ThogotoGP is subjected to DNA sequencing to confirm the integrity of the construct. BIV vector is then generated by co-transfecting 293 T cells with pCIgpSynSer (the packaging construct), pTracerA Rev (the Rev expression construct); pBIVfinalvec (the transfer vector construct), and pCIThogotoGP (the Thogoto virus envelope glycoprotein expression construct) by the methods described in Example 6.2. (Viral Vector Production). The BIV vectors pseudotyped with the Thogoto virus envelope are examined for titer, stability, and transduction efficiency in a variety of human and animal cells including human primary cells. The vectors are further tested for their ability to transduce retinal cells, neuronal cells as described in Example 13 and Example 14 respectively.

To enhance thogoto virus envelope glycoprotein expression efficiency in human cells, the coding sequence for the thogoto virus envelope glycoprotein is optimized (recoded). The recoded sequence is shown in SEQ ID NO:70.

A cell expressing a Thogoto virus envelope protein can be used as a packaging cell line for BIV vectors.

### Example 23

### Baculovirus Virus Envelope Pseudotyped Lentiviral Vector

Another viral envelope that my be used in the BIV vector system is the Baculovirus envelope protein. Preferably it is the GP64 derived from Autographa Californica virus. The DNA coding region of the GP64 region is cloned by techniques known to those in the art. This coding sequence is cloned into an expression construct compatible with the BIV system described above. For example, it is cloned into the pCi plasmid. This can be performed in a similar manner as described in Example 22 for the Thogoto envelope or Burns et al.(l993, PNAS. USA 90:8033-8037) for VSV-G.

A BIV viral vector containing the GP64 envelope is generated by co-transfecting 293 T cells with pCIgpSynSer (the packaging construct), pTracerA Rev (the Rev expression construct); pBIVfinalvec (the transfer vector construct), and pCIGP64 (the baculovirus envelope glycoprotein expression construct) by the methods described in Example 6.2.

This BIV viral vector with GP64 envelope is used to transduce both dividing and non-dividing primary RPE (ARPE) and HUVEC cells as generally described in Example 7. The results are shown in Table 6. Both non-dividing ARPE and HUVEC cells were transduced with the BIV vector at relatively high efficiency indicating that the vectors generated from the minimized BIV packaging and minimized transfer vector constructs are fully competent to mediate transgene expression in both dividing and non-dividing cells.

TABLE 6

Percentage of eGFP Positive Cells

| | | |
|---|---|---|
| Vector | Dividing | Nondividing |

ARPE

| | | |
|---|---|---|
| Mock infected | 0.1% (SD = 0.01) | 0.12% (SD = 0.1) |
| MLV viral vector | 88.2% (SD = 0.8) | 0.62% (SD = 0.3) |
| BIV viral vector | 93.3% (SD = 0.03) | 93% (SD = 1.1) |

HUVEC

| | | |
|---|---|---|
| Mock infected | 0.12% (SD = 0.02) | 0.11% (SD = 0.06) |
| MLV viral vector | 55.3% (SD = 1.0) | 0.6% (SD = 0.1) |
| BIV viral vector | 66.9% (SD = 1.7) | 53% (SD = 1.9) |

Also, BIV-GP64 particles were found to be stable based on their ability to endure ultracentrifugation.

The BIV-GP64 were also injected into the subretinal space of rats via subretinal injection. The finding suggest that P64 pseudotyped BIV viral vectors can be used to transduce retinal pigment epithelial cells in vivo.

A cell expressing a Baculovirus envelope protein can be used as a packaging cell line for BIV vectors.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

ATCC Accession No. 68092
ATCC Accession No. 68093
Bahnson et al., J. of Virol. Methods, 54:131-143 (1995)
Berkowitz, et al. J. of Virol., 7(7):3371-3382 (2001)
Beyer et al., J. Virol., 1:76(3):1488-95 (2002)
Burns et al., Proc. Natl. Acad. Sci., 90:8033-8037 (1993)
Carswell, S and Alwine, J. C., Mol. Cell Biol. 9,4248 (1989)
Cech, J.Amer.Med. Assn., 260:3030, (1988)
Dallinger, et al., J. Invest. Dermatol., 115(2):332 (2000)
Douglas, et al., Hum. Gene Ther., 12(4):401-413 (2001)
Freshney, Culture of Animal Cells, A Manual of Basic Techniques. (1994)
Garvey et al., Virology, 175:391-409 (1990)
Genbank Accession No. BC014127
Genbank Accession No. BC017153
Genbank Accession No. BC021911
Genbank Accession No. M32690
Genbank Accession No. NC_{—}001413
Genbank Accession No. NM_{—}138454
Genbank Accession No. XM_{—}134263
Gossen & Bujard, Proc. Natl. Acad. Sci., 89:5547-5551 (1992)
Hall et al., J Molec. App. Genet. 2, 101 (1983)
Helene, C., Anticancer Drug Design, 6(6):569, (1991).
Maher, et al., Antisense Res. and Dev., 1(3):227, (1991)
Marcus-Sakura, Anal.Biochem., 172:289, (1988)
Markowitz et al., J. Virol., 62:1120-1124; and Markowitz et al., Virology, 167:600-606, (1988)
Miyoshi, et al., J. Virol., 72:8150-8157 (1999)
O'Reilly et al., Cell, 88(2):277-85 (1997)
PCT Application No. PCT/US02/23868
Puttaraju et al., Nat. Biotechnol., 17(3):246-52 (1999)
Rigg et al., Virology, 218:290-295.
Sambrook et al., Molecular Cloning, A Laboratory Manual, 2^{nd} Ed., (1989)
U.S. Pat. No. 5,380,830.
U.S. Pat. No. 5,817,491, issued to Yee et al.
U.S. Pat. No. 6,277,633
Weintraub, Scientific American, 262:40, (1990)
WO 92/14829
WO 01/30843
WO 01/44458
WO 01/68835
WO 02/06463
WO 02/22663
WO 02/26827
WO 02/067970
WO 02/072851
WO 02/081513
Yee et al., Proc. Natl. Acad. Sci., 91:9563-9568 (1994)
Yu, et al., Proc. Natl. Acad. Sci., 83(10):3194-3198 (1986)

1. A recombinant lentiviral gene transfer system, comprising:
(a) (i) a packaging construct comprising a DNA segment comprising a promoter operably linked to a BIV gag gene and a BIV pol gene, or (ii) a first packaging construct comprising a DNA segment comprising a first promoter operably linked to a DNA segment comprising a BIV gag gene and a second packaging construct comprising a DNA segment comprising a BIV pol gene;
(b) a viral surface protein gene construct comprising a DNA segment comprising a promoter operably linked to a viral surface protein gene;
(c) a transfer vector construct comprising a DNA segment comprising a promoter operably linked to a first R region, a U5 region, a UTR region, a BIV packaging sequence, an RRE sequence, a promoter operably linked to a heterologous gene of interest, a 3′ polypurine tract region, a U3 region, and a second R region; and
(d) (i) a rev gene located on one of the packaging, viral surface protein gene, and transfer vector constructs or (ii) a rev construct comprising a DNA segment comprising a promoter operably linked to a rev gene.

2. The gene transfer system of claim 1, comprising a packaging construct comprising a DNA segment comprising a promoter operably linked to a BIV gag gene and a BIV pol gene.

3. The gene transfer system of claim 1, comprising a first packaging construct comprising a DNA segment comprising a first promoter operably linked to a DNA segment comprising a BIV gag gene and a second packaging construct comprising a DNA segment comprising a second promoter operably linked to a DNA segment comprising a BIV pol gene.

4. The gene transfer system of claim 2, wherein the packaging construct further comprises an RRE sequence.

5. The gene transfer system of claim 3, wherein at least one of the packaging constructs further comprises an RRE sequence.

6. The gene transfer system of claim 1, wherein the rev gene and RRE sequence are from BIV.

7. The gene transfer system of claim 2, wherein the gag gene comprises a recoded nucleotide sequence.

8. The gene transfer system of claim 2, wherein the gag and pol genes each comprise a recoded nucleotide sequence.

9. The gene transfer of claim 2, wherein the pol gene comprises a recoded nucleotide sequence.

10. The gene transfer system of claim 3, wherein the gag gene comprises a recoded nucleotide sequence.

11. The gene transfer system of claim 3, wherein the pol gene comprises a recoded nucleotide sequence.

12. The gene transfer system of claim 11, wherein the pol gene comprises an ATG start codon at 5′ end.

13. The gene transfer system of claim 1, wherein the protease region of the pol gene is mutated in the three amino acid motif of the catalytic center of the protease and wherein the mutated protease is less toxic to host cells when compared to a non-mutated BIV protease.

14. The gene transfer system of claim 13, wherein the protease region encodes a Thr to Ser mutation at amino acid 26 of the protease polypeptide.

15. The gene transfer system of claim 1, wherein the BIV packaging sequence comprises no more than the first 101 base pairs of the BIV gag gene open reading frame sequence.

16. The gene transfer of claim 15, wherein the packaging sequence consists essentially of the nucleotide sequence of SEQ ID NO:39.

17. The gene transfer system of claim 1, wherein the transfer vector construct comprises a DNA segment comprising a promoter operably linked to a first R region, a U5 region, a UTR region, a BIV packaging sequence, an RRE sequence, a promoter operably linked to a heterologous gene of interest, a 3′ polypurine tract region, a U3 region, a second R region, and a second U5 region.

18. The gene transfer system of claim 2, wherein the packaging construct further comprises the rev gene.

19. The gene transfer system of claim 1, wherein the viral surface protein gene construct comprises an env gene.

20. The gene transfer system of claim 19, wherein the env gene is selected from the group consisting of VSV-G env, LCMV env, LCMV-GP(WE-HPI)env, MOMLV env, Gibbon Ape Leukemia Virus (GaLV) env, an env gene from a member of the Phabdoviridae, an Alphavirus env gene, a Paramyxorivus env gene, a Flavivirus env gene, a Retrovirus env gene, an Arenavirus env gene, a Parainfluenza virus env gene, a Thogoto virus env gene, and a Baculovirus env gene.

21. The gene transfer system of claim 1, wherein the viral surface protein gene encodes VSV-G env.

22. The gene transfer system of claim 1, comprising a rev gene located on one of the packaging, viral surface protein gene, and transfer vector constructs.

23. The gene transfer system of claim 1, comprising a rev construct comprising a DNA segment comprising a promoter operably lined to a rev gene.

24. The gene transfer system of claim 6, wherein the rev gene does not include the native BIV rev intron.

25. The gene transfer system of claim 24, wherein the rev gene comprises SEQ ID NO:10.

26. The gene transfer system of claim 22, comprising an EF-1 promoter operably lined to the rev gene.

27. The gene transfer system of claim 23, wherein the promoter operably linked to the rev gene is the EF-1 promoter.

28. The gene transfer system of claim 26, wherein the RRE sequence consists essentially of the nucleic acid sequence of SEQ ID NO:40.

29. The gene transfer system of claim 1, wherein at least two of the promoters are the same.

30. The gene transfer system of claim 1, wherein all of the promoters are different.

31. The gene transfer system of claim 1, wherein at least one of the promoters is a regulatable promoter.

32. The gene transfer system of claim 1, which does not contain a cPPT.

33. The gene transfer system of claim 1, wherein the transfer vector construct further comprises a cPPT.

34. The gene transfer system of claim 33, wherein the cPPT is the cPPT from Human Immunodeficiency Virus.

35. The gene transfer system of claim 33, wherein the cPPT is a BIV cPPT.

36. The gene transfer system of claim 35, wherein the cPPT consists essentially of 535 base pairs corresponding to the nucleotides from base pairs 4758 to 5293 inclusive of SEQ ID NO:1.

37. The gene transfer system of claim 1, wherein the U3 region comprises an enhancer of polyadenylation.

38. The gene transfer system of claim 37, wherein the enhancer of polyadenylation consists essentially of the SV40 late polyadenylation enhancer element.

39. The gene transfer system of claim 1, which does not encode at least one of the vif, vpw, vpy, or tat genes of BIV.

40. The gene transfer system of claim 1, which does not encode the vif, vpw, vpy, tmx, and tat genes of BIV.

41. The gene transfer system of claim 1, wherein one or more nucleotides in the U3 region are altered or deleted such that U3 mediated transcription is diminished or abolished.

42. The gene transfer system of claim 1, comprising a woodchuck hepatitis virus regulatory response element operably linked to the heterologous gene of interest.

43. The gene transfer system of claim 1, wherein the heterologous gene of interest encodes a polypeptide selected from the group consisting of: T2-TrpRS, an Eph B receptor, an ephrin B ligand, a Fibrinogen E fragment, a soluble receptor for VEGF, angiostatin, endostain, optineurin, trabecular meshwork protein, a Rod-derived Cone Viability Factor (RdCVF) and an anti-apoptotic gene product.

44. The gene transfer system of claim 1, wherein the heterologous gene of interest encodes an RdCVF polypeptide selected from the group consisting of: SEQ ID NO: 61, SEQ ID NO:63, SEQ ID NO:65 and SEQ ID NO:67.

45. A producer cell comprising the gene transfer system of claim 1.

46. The producer cell of claim 45, wherein the gene transfer system is stably integrated into the producer cell's genome.

47. The producer cell of claim 45, wherein the gene transfer system is transiently transfected into the producer cell.

48. A method of producing replication-defective lentiviral particles, comprising:
(a) growing the producer cell of claim 45 in cell culture media under cell culture conditions sufficient to allow production of replication-defective lentiviral vector particles by the cell; and
(b) collecting said replication-defective lentiviral vector particles from the media.

49. A method according to claim 48, which further comprises adding a histone deacetylase inhibitor to the media.

50. A method according to claim 49, wherein the histone deacetylase inhibitor is butyric acid.

51. A replication-defective lentiviral particle produced according to the method of claim 48.

52. A method of treating or preventing a disease in an animal which has or is at risk of contracting said disease, comprising infecting one or more cells of the animal with a replication deficient recombinant lentiviral vector particle according to claim 51, wherein the heterologous gene of interest encodes a therapeutic product that is effective in treating or preventing said disease.

53. The method of claim 52, wherein the animal is a human.

54. The method of claim 52, wherein the one or more cells are ocular cells.

55. The method of claim 54, wherein the disease is selected from the group consisting of: ocular neovascularization, wet AMD (age related macular degeneration), diabetic proliferative retinopathy, non-diabetic retinopathy, diabetic macular edema, branch vein occlusion, central retinal vein occlusion, retinopathy in premature infants, rubeosis iridis, neovascular glaucoma, perifoveal telangiectasis, sickle cell retinopathy, Eale's disease, retinal vasculitis, Von Hippel Lindau disease, radiation retinopathy, retinal cryoinjury, retinitis pigmentosa, retinochoroidal coloboma, corneal neovascularization due to herpes simplex keratitis, corneal ulcers, keratoplasty, terigyia, or traumaretinal dystrophy, pathological aging, retinitis pigmentosa, Bardet-Biedel syndrome, Bassen-kornzweig syndrome, Best disease, choroidema, gyrate atrophy, congenital amourosis, Refsun syndrome, Stargardt disease and Usher syndrome.

56. The method of claim 55, wherein the therapeutic product is selected from the group consisting of: T2-TrpRS, an Eph B receptor, an ephrin B ligand, a Fibrinogen E fragment, a soluble receptor for VEGF, angiostatin, endostatin, optineurin, trabecular meshwork protein, a Rod-derived Cone Viability Factor (Rdcvf) and anti-apoptotic gene product.

57. The method of claim 55, wherein the therapeutic product is an Rdcvf polypeptide selected from the group consisting of: SEQ ID NO:61, SEQ ID NO:65 and SEQ ID NO:67.

58. The method of claim 52, wherein the disease is selected from the group consisting of: cancer, graft versus disease associated with allogeneic bone marrow transplant, and a neurologic disease.

59. The method of claim 52, wherein the one or more cells are infected in vivo.

60. The method of claim 52, wherein the one or more cells are infected in vitro.

61. A method of transducing cells in vitro with a recombinant lentiviral vector particle, comprising contacting the cells with the recombinant lentiviral vector particle according to claim 51, whereby the cells are transduced.

62. A method of transducing cells in vitro with a recombinant lentiviral vector particle, comprising contacting the cells with the recombinant lentiviral vector particle according to claim 51, whereby the cells are transduced.

63. A method of expressing a heterologous gene of interest in a cell which comprises transducing the cell with the recombinant lentiviral vector particle according to claim 51, whereby the heterologous gene of interest is expressed in the cell.

64. A packaging cell, comprising:
(a) (i) a packaging construct comprising a DNA segment comprising a promoter operably linked to a BIV gag gene and a BIV pol gene, or (ii) a first packaging construct comprising a DNA segment comprising a first promoter operably linked to a DNA segment comprising a BIV gag gene and a second packaging construct comprising a DNA segment comprising a second promoter operably linked to a DNA segment comprising a BIV pol gene.
(b) A viral surface protein gene construct comprising a DNA segment comprising a promoter operably linked to a viral surface protein gene; and
(c) (i) a rev gene located on one of the packaging, viral surface protein gene, and a transfer vector constructs or (ii) a rev construct comprising a DNA segment comprising a promoter operably linked to a rev gene.

65. The packaging cell of claim 64, comprising a packaging construct comprising a DNA segment comprising a promoter operably linked to a BIV gag gene and a BIV pol gene.

66. The packaging cell of claim 64, comprising a first packaging construct comprising a DNA segment comprising first promoter operably linked to a DNA segment comprising a BIV gag gene and a second packaging construct comprising a DNA segment comprising a second promoter operably linked to a DNA segment comprising a BIV pol gene.

67. The packaging cell of claim 65, wherein the gag gene comprises a recoded nucleotide sequence.

68. The packaging cell of claim 65, wherein the gag and pol genes each comprise a recoded nucleotide sequence.

69. The packaging cell of claim 65, wherein the pol gene comprises a recoded nucleotide sequence.

70. The packaging cell of claim 66, wherein the gag gene comprises a recoded nucleotide sequence.

71. The packaging cell of claim 66, wherein the pol gene comprises a recoded nucleotide sequence.

72. The packaging cell of claim 64, wherein the protease region of the pol gene is mutated in the three amino acid motif of the catalytic center of the protease and wherein the mutated protease is less toxic to host cells when compared to a non-mutated BIV protease.

73. The packaging cell of claim 72, wherein the protease region encodes a Thr to Ser mutation at amino acid 26 of the protease polypeptide.

74. The packaging cell of claim 64, wherein the viral surface protein gene construct comprises an env gene.

75. The packaging cell of claim 74, wherein the env gene is selected from the group consisting of VSV-G env, LCMV env, LCMV-GP(WE-HPI)env, MoMLV env, Gibbon Ape Leukemia Virus (GaLV) env, an env gene from a member of the Phabdoviridae, an Alphavirus env gene, a Paramyxovirus env gene, a Flavivirus env gene, a Retrovirus env gene, an Arenavirus env gene and a Parainfluenza virus env gene.

76. The packaging cell of claim 64, wherein the viral surface protein gene encodes VSV-G env.

77. The packaging cell of claim 64, comprising a rev gene located on one of the packaging, viral surface protein gene, and transfer vector constructs.

78. The packaging cell of claim 64, comprising a rev construct comprising a DNA segment comprising a promoter operably linked to a rev gene.

79. The packaging cell of claim 64, wherein the rev gene is from BIV but does not include the native BIV rev intron.

80. The packaging cell of claim 79, wherein the rev gene comprises SEQ ID NO:10.

81. The packaging cell of claim 77, comprising an EF-1 promoter operably lined to the rev gene.

82. The packaging cell of claim 78, wherein the promoter operably linked to the rev gene is the EF-1 promoter.

83. The packaging cell of claim 64, wherein at least two of the promoters are the same.

84. The packaging cell of claim 64, wherein all of the promoters are different.

85. The packaging cell of claim 86, wherein the cell is selected from the group consisting of a 293 cell, a 293 T cell, a COS cell, a HeLa cell, and a Cf2TH cell.

86. An isolated BIV POL protein, comprising an amino acid sequence at least 90% identical to the amino acid sequence shown in SEQ ID NO:51.

87. The isolated BIV POL protein of claim 86, comprising SEQ ID NO:51.

88. The isolated BIV POL protein of claim 86, comprising a methionine at the N-terminus of said POL protein.

89. An isolated nucleic acid molecule comprising a nucleotide sequence encoding the BIV POL protein of claim 86.

90. An isolated nucleic acid molecule comprising a nucleotide sequence encoding the BIV POL protein of claim 87, wherein said nucleotide sequence consists essentially of SEQ ID NO:50.

91. An isolated nucleic acid molecule comprising a nucleotide sequence encoding the BIV POL protein of claim 88, wherein said nucleotide sequence consists essentially of SEQ ID NO:53.

92. An isolated nucleic acid molecule comprising a minimal BIV packaging sequence, wherein said minimal BIV packaging sequence is at least 90% identical to the nucleotide sequence set forth in SEQ ID NO:39.

93. The isolated nucleic acid molecule of claim 92, wherein the minimal BIV packaging sequence consists essentially of the nucleotide sequence set forth in SEQ ID NO:39.

94. An isolated nucleic acid molecule comprising a nucleotide sequence encoding a BIV REV protein, wherein said nucleotide sequence encodes an amino acid sequence at least 90% identical to the amino acid sequence encoded by the nucleotide sequence set forth in SEQ ID NO:10.

95. The isolated nucleic acid molecule of claim 94, wherein the nucleotide sequence encoding the BIV REV protein encodes the same amino acid sequence encoded by the nucleotide sequence set forth in SEQ ID NO:10.

96. The isolated nucleic acid molecule of claim 94, wherein the nucleotide sequence is at least 90% identical to the nucleotide sequence set forth in SEQ ID NO:10.

97. The isolated nucleic acid molecule of claim 94, wherein the nucleotide sequence consists essentially of the nucleotide sequence set forth in SEQ ID NO:10.

98. An isolated nucleic acid molecule comprising a minimal BIV RRE sequence, wherein said minimal BIV RRE sequence is at least 90% identical to the nucleotide sequence set forth in SEQ ID NO:40.

99. The isolated nucleic acid molecule of claim 98, wherein the minimal BIV RRE sequence consists essentially of the nucleotide sequence set forth in SEQ ID NO:40.


SEQUENCE LISTING (71 sequences)

SEQ ID NO: 1 - DNA, 8960 bp, Bovine immunodeficiency virus
tgtggggcagggtgggacctcaggacaacagcagcccccggacttcccatatgtgaattggactggatccagggaacaaaataacccagaagggggattagactctggggcttggtatgaaggcctgagaggttctcagtagattgtaagtcttcggcgagactgcatgtctgcacgtagacaggaaatgtttatcttctcagctgattgtggttaggccgattactggaaactagacaacctgattcattagtggttaagattatgcataagtgctcgcaatgatgtagctgcttacgcttgcttactccgccctgaaacgcctaccttaacacgcaacacgcccacctgtaagaatatataaaccatatcttcactctgtacttcagctcgtgtagctcattagctccgagctccccaacctacagcctgagaggcactggctcggttgggtagccagcctttcgggtaataaaggcttgttggcattcggcatctacccgtgcctcctgtcttgtcttactcgagcgaacccacaactccgtcctgctgagctcacagctcgcggggcggtgaagaacacccaacagttggcgcccaacgtggggctcgagtaagagagactcggctcgagtaaaagaagacccagctcgaacgagaagactccggacaggtgagtagttgcgtgttttccccgggatgaagagaagggagttagaaaagaagcttcgtaaggttagggtgacaccccaacaggataaatattatactatagggaatcttcaatgggccattagaatgataaatctaatggggatcaaatgtgtgtgtgacgaggagtgctcggcagcagaggtagcccttatcataacccaattttcagctttagacttagaaaattctcctatcagaggtaaggaggaggtggccataaaaaatactctgaaggttttctggtccctgctggcggggtacaaaccagagagtacagaaacggccctaggatattgggaggcctttacatatagagaaagggaggccagagctgataaggaaggcgaaattaagagtatttacccttccctaacacagaacacacagaataagaagcagacatcgaatcagacaaacactcaatcattaccagctatcactactcaagatggtactcctaggtttgatcctgacctcatgaagcagcttaagatctggtcagacgccactgaaagaaatggggttgaccttcatgcagtgaatatattaggggtcattacagcaaacctagtacaggaagaaattaaactcctcttgaatagtacacccaagtggagattagatgtacaacttatagaatcaaaagtaagagagaaagaaaatgcccacagaacgtggaaacagcatcatccagaagccccaaaaacagatgaaatcatcggtaaggggcttagttctgctgaacaagccaccctgatctcagtagaatgcagagaaactttcagacagtgggtgctgcaggcagctatggaggtggcacaggcaaaacatgctaccccaggtcccatcaacattcatcagggacccaaggagccgtacacagactttataaatagattagtggcagcccttgaaggtatggcggctccagaaaccacaaaagaatacttactccaacatctatctattgatcatgccaatgaagactgccagtctattctaagacctttgggacccaacaccccaatggagaaaaaattagaagcatgtagggtagtgggatctcagaaatcaaagatgcaatttttggtagcagctatgaaagaaatggggatccaatcaccaattccagcagtcttgcctcacacaccagaagcatatgcctcccaaacctcagggcccgaggatggtaggagatgttacggatgtgggaagacaggacatttgaagaggaattgtaaacagcaaaaatgctaccattgtggcaaacctggccaccaagcaagaaactgcaggtcaaaaaacgggaagtgctcctctgccccttatgggcagaggagccaaccacagaacaattttcaccagagcaacatgagttctgtgaccccatctgcaccccctcttatattagattagacaaacagccttttataaaggtgttcatagggggaagatgggtaaaagggttagtagacactggagcagatgaggtagtgcttaagaacatacattgggataggataaaagggtatccagggacaccaattaaacaaattggggtaaatggagtaaatgtggccaaaaggaagacccacgtagagtggagatttaaggataagactgggataattgatgtcttgttctcagatactcctgtaaacctttttgggagatctcttctacgtagcatagtgacttgcttcaccctacttgttcacacagaaaaaatcgaacccctacccgtcaaggtaaggggaccagggcctaaggtaccccagtggcccttgacaaaagaaaagtatcaggctcttaaggaaattgtgaaagatcttttagcagaaggaaaaatttccgaagctgcttgggataacccatataataccccagtttttgttataaagaaaaagggaacgggaagatggaggatgctaatggattttagggaattaaataagataacagttaaaggacaagaattctctacaggcttaccttaccctccaggaattaaggaatgtgaacacttaactgcaatagatataaaagatgcctactttactatccctttacatgaggactttagaccctttacagccttctctgtagtccctgtaaatcgagaaggacctatagagaggttccagtggaatgttctaccacaaggatgggtatgtagccctgccatttatcagactaccacccagaagattatagaaaacattaaaaagagtcacccagatgtcatgttgtatcaatatatggatgatttgttgattgggtctaatagggatgatcataagcaaatagtgcaggaaatcagggataagttaggatcatatggtttcaagactccagatgaaaaggtccaggaagagagagtgaaatggatcggttttgagctcacacccaagaaatggcgttttcagcccaggcaactaaagataaaaaacccactcacagtaaatgaattacagcaattagtaggtaattgtgtttgggtacagccagaagtaaaaatccctctataccccttaaccgatctactgagggataagaccaatctccaagaaaagatacaactaacaccagaagccatcaagtgtgtagaagaattcaatctaaaactaaaagatccagaatggaaagatagaataagagaaggagcagaattagtcataaaaatacagatggttcctcggggcatagtatttgatctgttgcaagatggaaatcccatatggggaggagtaaaaggactaaattatgatcattcaaacaaaataaaaaagatacttagaactatgaatgagctgaacagaacagtggtaattatgacaggaagagaagctagtttcctgcttcctgggtcttctgaagattgggaagcggcactccagaaggaagaaagtctaacacaaatattcccagtaaagttttataggcactcctgcagatggacctccatatgtgggccagtaagagaaaatctaaccacctactatactgacggagggaagaaagggaaaacagctgcagcagtatattggtgtgaaggaaggactaagtcaaaggtatttccaggaaccaatcaacaggcggaattgaaggccatatgcatggctctcttggatggaccaccaaaaatgaatatcataacagatagtagatacgcctatgagggaatgagagaagaaccagaaacgtgggccagggaaggaatctggctggagattgccaagatattgccctttaagcagtacgtgggggtcgggtgggtgcctgcacataaagggataggaggaaatacagaggcagatgaaggagttaagaaagccttagaacagatggccccgtgtagccctcctgaggccattctattaaaaccaggagaaaaacaaaatctggagacagggatctacatgcaggggcttagaccacaaagcttcctcccaagagcagacttaccagtagccatcacaggaaccatggtagattcagagctacagctacagctacttaacataggaactgagcatataagaatccaaaaagatgaggtcttcatgacctgtttcctagaaaatatcccctcagccactgaagatcatgagagatggcatacctcaccagacattttggttaggcagttccatctccctaagagaatagctaaagagatagtagccagatgccaagaatgtaaaaggacaaccactagcccagtcagaggaacaaaccccagaggtcgattcttatggcagatggacaatactcactggaataaaacaattatttgggtagcagtagagacaaattcaggattagtggaagctcaggtgatccctgaagaaacagcactacaagtagctctctgcattttacagctaatccagagatatacagttcttcacttacatagtgacaacgggccgtgctttactgcacacaggatagaaaatctatgtaagtatctggggatcacaaaaactacgggaataccctacaacccacaatcccagggagttgtagaaagagcccacagagatctaaaagacagattggcagcttatcagggagattgtgaaaccgtagaagcagcccttagcctcgcattagtttctttaaataaaaaaagagggggaatagggggccatacaccatatgaaatatacctagaatcagaacataccaaataccaagaccaactagaacaacaattttcaaaacaaaaaattgaaaagtggtgttacgtaaggaacagaagaaaggaatggaaaggaccctacaaagtgttgtgggacggagacggggcagcagtaatagaggaagagggaaaaacagccttatatccacaccgtcatatgcgcttcatcccccccccagattcagatatccaagatgggagttcgtgaggcagacagaatacagcatgaccgcgtgcgtaagaaaagggaaattagtccttacttaccagtacgcgatctggaaaagagtctggacgatagaaacaggatttacagatccaagtctgtttatgaccccagctggaacacacaccactgaagaaataggtcacttagatctcttttggcttaggtactgttcatgtccgcatgagatgcccccgtggctagacttccttagaggcaccctcaatctacgcatttcctgtcgacgcgctcttcaagcgtcagtgttgactagcacccctagacactccctccaacgcttagctgcacttcagctgtgcactaacgcatgtctctgttggtacccgttaggacgcatcaacgacaccaccccgttgtggttgaacttttcgtctgggaaggaaccaacgatccaacaactgagtggccacccctaactcgtcgtaacattcatagattgtggcaatatgcccggaccttgggtggcgatgataatgttgccacagcccaaagaaagctttggaggaaagccaattggctggcttttctggaacacgtgcaaaggacctaggcgggactgtccacattgttgttgtcccatatgtagttggcattgtcagctttgctttttgcagaaaaatctaggaatcaactatggatcaggacctagacggcgcggaacgcggggaaaggggaggaggatccgaagaactgcttcaggaggagatcaacgaagggaggctgacagccagagaagctttacaaacatggatcaataacggtgagatccacccttgggtcctggcaggaatgctgtccatgggagtaggaatgctactaggagtatattgtcagttaccagacacactgatttggatactaatgtttcaattatgcctttattggggtttgggtgaaacatctagagaattagacaaggatagttggcagtgggtcagaagtgtatttataatagcaatattgggaactctcactatggcaggaactgctttggccgacgacgatcaaagtactttaatccccaatatcacaaaaattcctacaaaggacacggaacccggttgcacctatccgtggatattaatcctcttgattttggctttcatactgggaattctgggtataatacttgtcttgagacgcagcaactcggaggatatattggcagccagagataccatagattggtggctctcagctaatcaggaaatacctccaaagtttgctttcccaataatattaatatcttcccctctagcaggcataataggatattatgtcatggaaaggcacttagagatcttcaaaaagggatgtcaaatttgtgggagcctgagcagcatgtggggaatgcttttggaagaaattggcaggtggctcgcacgtagggaatggaatgttagtagagtaatggttatcctcttaatcagcttcagttggggaatgtatgtcaatagggtaaatgcctcagggtcacatgtagccatggtcaccagccctccagggtaccgcatagtgaatgataccagccaggcaccttggtattgcttctcctcggcaccaatcccaacgtgtagttcctctcagtggggagacaaatattttgaggagaaaataaacgagacactggtcaaacaggtgtatgaacaggccgcgaaacattcgagagccacatggattgaacctgatctattggaggaagcagtctatgagctagctctgttatcagctaatgacagtcgtcaggtggtggtagaaaatggtacagacgtatgtagctcacagaactcgagcacaaacaaaggccacccaatgacgcttctaaagttgagagggcaggtgtcagaaacttggatagggaattcctccctccagttttgtgtccagtggccatatgtcttggtaggtcttaataatagtgatagtaatattagcttcaattcgggagattggatagcaaccaattgtatgcacccaattacactaaataaaagtgcacaagatctaggaaaaaattttccgagactaacatttcttgacggacaactgtcccagttgaagaacacactgtgcggacataacacaaactgtttgaaatttggaaacaagtccttcagtacaaattccctaatactatgccaagacaaccccatcggcaacgacaccttttatagcctaagtcattccttctcaaaacaggcctctgcccggtggattcttgtaaaggtccccagctatgggtttgtggtagtaaatgacacagatacaccaccatccctccgcatccgaaagcctcgagcagtcggactagcaatattcctgcttgtgctggctatcatggccatcacatcctccttggtggcagctacaacgctcgtgaaccagcacacgacggctaaggttgtggagagggttgtgcaaaatgtgtcatatattgctcaaacccaggaccaattcacccacctgttcaggaatataaacaacagattaaatgtcctacaccatagagtttcatacttggagtatgtagaggaaatcagacaaaaacaagtattctttggttgcaaacctcatggaaggtattgccactttgactttggaccagaggaagttggatggaacaatagttggaatagcaaaacttggaatgatctacaagatgagtatgataagatagaagaaaaaatattaaaaattcgagtggactggctcaatagctccctgagtgacacacaggacacctttggcctggagacctctatttttgaccatttagtgcaattgtttgattggacttcttggaaagactggataaaaatcattatagtaatcattgtactttggcttctgataaagattctcctaggtatgttaagaagctgcgccaaggtcagccagaattaccaacatctcccggcggaggaggaggacggggacacagagccagaaagctccccggcgagaggagacccggcttctggaagtctctacgagaattggttgaacaaaataggagaaagcaagaacgacgcctatcgggtctggacagaagaatacaacagcttgaggatcttgttcgccacatgtcgctgggatctcctgacccctcaactccttcagcttccgttctttctgttaaccctcctgctcaaactcctttgggacatcttccgccacgctcctattttaaacttaaaagggtggactgtggggcagggtgggacctcaggacaacagcagcccccggacttcccatatgtgaattggactggatccagggaacaaaataacccagaagggggattagactctggggcttggtatgaaggcctgagaggttctcagtagattgtaagtcttcggcgagactgcatgtctgcacgtagacaggaaatgtttatcttctcagctgattgtggttaggccgattactggaaactagacaacctgattcattagtggttaagattatgcataagtgctcgcaatgatgtagctgcttacgcttgcttactccgccctgaaacgcctaccttaacacgcaacacgcccacctgtaagaatatataaaccatatcttcactctgtacttcagctcgtgtagctcattagctccgagctccccaacctacagcctgagaggcactggctcggttgggtagccagcctttcgggtaataaaggcttgttggcattcggcatctacccgtgcctcctgtcttgtcttactcgagcgaacccacaactccgtcctgctgagctcacagctcgcggggcggtgaagaacacccaaca

SEQ ID NO: 2 - DNA, 32 bp, Artificial Sequence
  Primer RRE65′NotI
aaagcggccgctccggtggattcttgtaaagg

SEQ ID NO: 3 - DNA, 33 bp, Artificial Sequence
  Primer RRE63′NotI
aaagcggccgcggcgcctccaagtatgaaactc

SEQ ID NO: 4 - DNA, 36 bp, Artificial Sequence
  Primer Rev15Afl3
ggacgcgtcgactctagatctaggaatcaactatgg

SEQ ID NO: 5 - DNA, 40 bp, Artificial Sequence
  Primer Rev23Agel2
tttaccggtcgcgagcttagcttacaatctactgagaacc

SEQ ID NO: 6 - DNA, 24 bp, Artificial Sequence
  Primer Rev155868
gttctagatggctggcttttctgg

SEQ ID NO: 7 - DNA, 25 bp, Artificial Sequence
  Primer Rev13
gagaatcgttattgatccatgtttg

SEQ ID NO: 8 - DNA, 28 bp, Artificial Sequence
  Primer Rev25
ggatcaataacgattctcctaggtatgt

SEQ ID NO: 9 - DNA, 26 bp, Artificial Sequence
  Primer Rev237526
ttactagtggttattttgttccctgg

SEQ ID NO: 10 - DNA, 561 bp, Bovine immunodeficiency virus
  misc_feature: Rev gene
atggatcaggacctagacggcgcggaacgcggggaaaggggaggaggatccgaagaactgcttcaggaggagatcaacgaagggaggctgacagccagagaagctttacaaacatggatcaataacgattctcctaggtatgttaagaagctgcgccaaggtcagccagaattaccaacatctcccggcggaggaggaggacggggacacagagccagaaagctccccggcgagaggagacccggcttctggaagtctctacgagaattggttgaacaaaataggagaaagcaagaacgacgcctatcgggtctggacagaagaatacaacagcttgaggatcttgttcgccacatgtcgctgggatctcctgacccctcaactccttcagcttccgttctttctgttaaccctcctgctcaaactcctttgggacatcttccgccacgctcctattttaaacttaaaagggtggactgtggggcagggtgggacctcaggacaacagcagcccccggacttcccatatgtgaattggactggatccagggaacaaaataa

SEQ ID NO: 11 - DNA, 48 bp, Artificial Sequence
  Primer WPRE5
gagctgtacaagtaaagcggccaaccctcctgcagaaactcctttggg

SEQ ID NO: 12 - DNA, 29 bp, Artificial Sequence
  Primer WPRE3
ggaacaaaagctgggtaccgggccccccc

SEQ ID NO: 13 - DNA, 33 bp, Artificial Sequence
  Primer RRE1
gttggcgcccaacgtggggctcgagtaagagag

SEQ ID NO: 14 - DNA, 27 bp, Artificial Sequence
  Primer RRE2
agatctgaattctaagtgacctatttc

SEQ ID NO: 15 - DNA, 29 bp, Artificial Sequence
  Primer RRE3
gaattcagatcttatgggaatgaaagacc

SEQ ID NO: 16 - DNA, 27 bp, Artificial Sequence
  Primer RRE4
aactgctgagggcgggaccgcatctgg

SEQ ID NO: 17 - DNA, 30 bp, artificial sequence
  PCR primer 5′GAGRRE1
ggcgaattcgatctaggaaaaaattttccg

SEQ ID NO: 18 - DNA, 27 bp, artificial sequence
  PCR primer 3′GAGRRE1
ggaagatctccacaaacccatagctgg

SEQ ID NO: 19 - DNA, 22 bp, artificial sequence
  PCR primer 5′GAGRRE2
cccgaattcaaaggtccccagc

SEQ ID NO: 20 - DNA, 25 bp, artificial sequence
  PCR primer 3′GAGRRE2
ggaagatctctctatggtgtaggac

SEQ ID NO: 21 - DNA, 26 bp, artificial sequence
  PCR primer 5′GAGRRE3
ccggaattcgagtttcatacttggag

SEQ ID NO: 22 - DNA, 23 bp, artificial sequence
  PCR primer 3′GAGRRE3
ggaagatcttgcactaaatggtc

SEQ ID NO: 23 - DNA, 23 bp, artificial sequence
  PCR primer 5′GAGRRE4
ccggaattccctaatactatgcc

SEQ ID NO: 24 - DNA, 25 bp, artificial sequence
  PCR primer 3′GAGRRE4
ggaagatctcttagccgtcgtgtgc

SEQ ID NO: 25 - DNA, 26 bp, artificial sequence
  PCR primer 5′GAGRRE5
ggcgaattcgggttgtgcaaaatgtg

SEQ ID NO: 26 - DNA, 25 bp, artificial sequence
  PCR primer 3′GAGRRE5
cctagatctcattccaagttttgct

SEQ ID NO: 27 - DNA, 26 bp, artificial sequence
  PCR primer 5′GAGRRE6
ccggaattcgtggattcttgtaaagg

SEQ ID NO: 28 - DNA, 26 bp, artificial sequence
  PCR primer 3′GAGRRE6
ggaagatctctccaagtatgaaactc

SEQ ID NO: 29 - DNA, 24 bp, artificial sequence
  PCR primer 5′GAGRRE7
ccagaattccaccaccatccctcc

SEQ ID NO: 30 - DNA, 24 bp, artificial sequence
  PCR primer 3′GAGRRE7
ggaagatctcaaccaaagaatact

SEQ ID NO: 31 - DNA, 26 bp, Artificial Sequence
  PCR primer NRS1
aacagttggcgcccaacgtggggctc

SEQ ID NO: 32 - DNA, 34 bp, Artificial Sequence
  PCR primer NRS2
atgcatcacgtgggtgtcaccctaaccttacgaa

SEQ ID NO: 33 - DNA, 33 bp, Artificial Sequence
  PCR primer NRS3
cacgtgatgcatcgatctaaaagacagattggc

SEQ ID NO: 34 - DNA, 29 bp, Artificial Sequence
  PCR primer NRS4
cataagatctgaattcaatgatctaagtg

SEQ ID NO: 35 - DNA, 36 bp, artificial sequence
  PCR primer NRS32
atgcatcacgtgattctaatggcccattgaagattc

SEQ ID NO: 36 - DNA, 33 bp, artificial sequence; identical to SEQ ID NO: 33
  PCR primer NRS33

SEQ ID NO: 37 - DNA, 31 bp, artificial sequence
  QuickChange reaction primer KOATAG Forward
gcgtgttttccccggggtgaagagaagggag

SEQ ID NO: 38 - DNA, 31 bp, artificial sequence
  QuickChange reaction primer KOATAG Reverse
ctcccttctcttcaccccggggaaaacacgc

SEQ ID NO: 39 - DNA, 210 bp, Artificial Sequence
  BIV packaging signal sequence
gttggcgcccaacgtggggctgagtaagagagactcggctcgagtaaaagaagacccagctcgaacgagaagactccggacaggtgagtagttgcgtgttttccccggggtgaagagaagggagttagaaaagaagcttcgtaaggttagggtgacaccccaacaggataaatattatactatagggaatcttcaatgggccattagaat

SEQ ID NO: 40 - DNA, 312 bp, Artificial Sequence
  312 bp of BIV env sequence contains BIV RRE sequence
gtggattcttgtaaaggtccccagctatgggtttgtggtagtaaatgacacagatacaccaccatccctccgcatccgaaagcctcgagcagtcggactagcaatattcctgcttgtgctggctatcatggccatcacatcctccttggtggcagctacaacgctcgtgaaccagcacacgacggctaaggttgtggagagggttgtgcaaaatgtgtcatatattgctcaaacccaggaccaattcacccacctgttcaggaatataaacaacagattaaatgtcctacaccatagagtttcatacttgga

SEQ ID NO: 41 - RNA, 7 nt, avian sarcoma/leukosis virus
aaauuua

SEQ ID NO: 42 - RNA, 7 nt, Human immunodeficiency virus
uuuuuua

SEQ ID NO: 43 - RNA, 7 nt, mouse mammary tumor virus
aaaaaac

SEQ ID NO: 44 - RNA, 7 nt, feline immunodeficiency virus
gggaaac

SEQ ID NO: 45 - DNA, 7 bp, Bovine Immunodeficiency virus
caaaaat

SEQ ID NO: 46 - DNA, 7 bp, Bovine Immunodeficiency virus
aaaaaac

SEQ ID NO: 47 - DNA, 48 bp, Bovine Immunodeficiency virus
aaaaaacgggaagtgctcctctgccccttatgggcagaggagccaacc

SEQ ID NO: 48 - RNA, 48 nt, Bovine immunodeficiency virus
aaaaaacgggaagugcuccucugccccuuaugggcagaggagccaacc

SEQ ID NO: 49 - DNA, 4427 bp, Artificial Sequence
  DNA sequence of recoded BIV gag/pol
atgaagcggagagagctggagaagaaactgaggaaagtgcgcgtgacacctcaacaggacaagtactataccatcggcaacctgcagtgggccatccgcatgatcaacctgatgggcatcaagtgcgtgtgcgacgaggaatgcagcgccgctgaggtcgccctgatcatcacccagtttagcgccctcgacctggagaactcccctatccgcggcaaggaagaggtggccatcaagaataccctgaaggtgttttggagcctgctggccggatacaagcctgagagcaccgagaccgccctgggatactgggaagccttcacctacagagagagggaagctagagccgacaaggagggagagatcaagagcatctaccctagcctgacccagaacacccagaacaagaaacagaccagcaatcagacaaacacccagagcctgcccgctatcaccacacaggatggcacccctcgcttcgaccccgacctgatgaagcagctgaagatctggtccgatgccacagagcgcaatggagtggacctgcatgccgtgaacatcctgggagtgatcacagccaacctggtgcaagaagagatcaagctcctgctgaatagcacacccaagtggcgcctggacgtgcagctgatcgagagcaaagtgagagagaaggagaacgcccaccgcacctggaagcagcatcaccctgaggctcccaagacagacgagatcattggaaagggactgagctccgccgagcaggctaccctgatcagcgtggagtgcagagagaccttccgccagtgggtgctgcaggctgccatggaggtcgcccaggctaagcacgccacacccggacctatcaacatccatcaaggccctaaggaaccctacaccgacttcatcaaccgcctggtggctgccctggaaggaatggccgctcccgagaccacaaaggagtacctcctgcagcacctgagcatcgaccacgccaacgaggactgtcagtccatcctgcgccctctgggacccaacacacctatggagaagaaactggaggcctgtcgcgtggtgggaagccagaagagcaagatgcagttcctggtggccgctatgaaggaaatggggatccagtctcctattccagccgtgctgcctcacacacccgaagcctacgcctcccaaacctcagggcccgaggatggtaggagatgttacggatgtgggaagacaggacatttgaagaggaattgtaaacagcaaaaatgctaccattgtggcaaacctggccaccaagcaagaaactgcaggtcaaaaaacgggaagtgctcctctgccccttatgggcagaggagccaaccacagaacaattttcaccagagcaacatgagttctgtgaccccatctgcaccccctcttatattagattagacaaacagccttttataaaggtgttcattggcggccgctgggtgaagggactggtggacacaggcgctgacgaggtggtgctgaagaacatccactgggaccgcatcaaaggctaccctggaacacccatcaagcagatcggcgtgaacggcgtgaacgtggctaagcgcaaaacacatgtggagtggagattcaaagacaagaccggcatcattgacgtcctcttcagcgacacacctgtgaacctgtttggcagaagcctgctcagatccatcgtgacctgctttaccctgctggtgcacaccgagaagatcgagccactgcctgtgaaggtgcgcggccctggacctaaggtgccacaatggcccctgaccaaggagaaataccaggccctgaaggagatcgtgaaggacctgctggccgagggaaagatcagcgaagctgcctgggacaacccttacaacacacccgtgttcgtgatcaagaagaaaggcaccggccgctggcgcatgctgatggacttccgcgagctgaataagatcaccgtgaaaggccaagagttcagcacaggactcccttatccacccggcatcaaggagtgtgagcacctgaccgccatcgacatcaaggacgcctacttcaccatccctctgcacgaggacttcagacccttcacagccttcagcgtggtcccagtgaaccgcgagggccccatcgagcgcttccagtggaacgtcctgcctcaaggctgggtgtgctcccctgccatctaccagaccacaacccagaagatcattgagaacatcaagaagagccatcccgacgtgatgctgtatcagtacatggatgacctcctgattggcagcaatcgcgatgaccacaagcagatcgtgcaggagatcagagacaagctgggcagctatggcttcaagacacccgacgagaaagtgcaggaagagcgcgtgaagtggatcggcttcgagctgacacctaagaaatggagattccagcctaggcaactgaagatcaagaacccactgaccgtgaacgaactccagcagctggtcggcaactgtgtgtgggtgcagcccgaggtgaagatccctctgtacccactgaccgatctgctccgcgacaagaccaacctgcaggaaaagatccagctgacacccgaggccatcaagtgcgtggaagagttcaacctgaagctgaaagatcccgagtggaaggacagaattcgcgaaggagccgagctggtgatcaagatccaaatggtccctcgcggcatcgtgttcgacctgctgcaagacggcaatcctatctggggaggcgtgaaaggactgaactacgaccacagcaacaagatcaagaagatcctgcgcaccatgaacgagctgaaccgcaccgtggtgatcatgaccggacgcgaagctagctttctcctgcctggatccagcgaggattgggaggccgccctgcagaaggaagagagcctgacccaaatctttcccgtgaagttctaccgccatagctgtagatggacaagcatctgtggacccgtccgcgagaacctgaccacctactataccgacggcgggaagaaaggaaagacagctgccgcagtgtactggtgtgaaggaagaactaagagcaaagtgttccctggaaccaatcaacaggctgagctgaaggcaatctgcatggctctgctggacggacctcccaagatgaacatcatcaccgacagccgctacgcttatgagggcatgagagaggaacctgagacctgggctcgcgagggcatctggctggagattgcaaagatcctgccattcaagcaatacgtcggagtgggctgggtccctgctcacaaaggcattggaggcaataccgaggctgacgaaggagtgaagaaagccctggagcaaatggcaccatgttcccctcccgaggctatcctgctcaaacctggcgagaagcaaaacctggagaccggcatctacatgcaaggcctgagacctcagagcttcctgccccgcgctgacctccctgtcgcaatcactggcaccatggtggactccgagctgcagctccaactgctgaacatcggcaccgagcacattcgcatccagaaggacgaggtgttcatgacatgcttcctggagaacatccctagcgccaccgaagaccacgagagatggcacacatccccagacatcctggtccgccagttccacctgcccaagcgcatcgccaaggagatcgtcgcccgctgccaggagtgcaagagaaccacaacctccccagtgcgcggcaccaaccctagaggacgcttcctgtggcagatggacaacacacactggaacaaaaccatcatttgggtcgcagtggagactaacagcggactggtggaggctcaggtgattcccgaagagaccgcactgcaagtggccctgtgtatcctccagctgatccaacgctacaccgtcctgcacctgcacagcgacaacggaccctgcttcacagctcaccgcatcgagaacctgtgcaagtacctgggcatcaccaagacaaccggcattccctacaatcctcagagccaaggagtcgtggaaagagcccatcgcgacctgaaggacagactggctgcctatcaaggcgactgcgagaccgtggaagctgcactgagcctcgccctggtcagcctgaacaagaagagaggaggcatcggcggacacacaccctacgagatctatctggagagcgagcacaccaagtatcaggaccaactggagcagcaattcagcaagcagaagatcgagaaatggtgctacgtccgcaacagacgcaaggagtggaagggcccttacaaggtgctgtgggatggcgacggagctgcagtgatcgaggaagagggcaagaccgctctgtatccccaccggcacatgcgcttcatcccacctcccgacagcgatatccaggacggctccagctga

SEQ ID NO: 50 - DNA, 3108 bp, Bovine immunodeficiency virus
  misc_feature: BIV Pol DNA Sequence
cgggaagtgctcctctgccccttatgggcagaggagccaaccacagaacaattttcaccagagcaacatgagttctgtgaccccatctgcaccccctcttatattagattagacaaacagccttttataaaggtgttcatagggggaagatgggtaaaagggttagtagacactggagcagatgaggtagtgcttaagaacatacattgggataggataaaagggtatccagggacaccaattaaacaaattggggtaaatggagtaaatgtggccaaaaggaagacccacgtagagtggagatttaaggataagactgggataattgatgtcttgttctcagatactcctgtaaacctttttgggagatctcttctacgtagcatagtgacttgcttcaccctacttgttcacacagaaaaaatcgaacccctacccgtcaaggtaaggggaccagggcctaaggtaccccagtggcccttgacaaaagaaaagtatcaggctcttaaggaaattgtgaaagatcttttagcagaaggaaaaatttccgaagctgcttgggataacccatataataccccagtttttgttataaagaaaaagggaacgggaagatggaggatgctaatggattttagggaattaaataagataacagttaaaggacaagaattctctacaggcttaccttaccctccaggaattaaggaatgtgaacacttaactgcaatagatataaaagatgcctactttactatccctttacatgaggactttagaccctttacagccttctctgtagtccctgtaaatcgagaaggacctatagagaggttccagtggaatgttctaccacaaggatgggtatgtagccctgccatttatcagactaccacccagaagattatagaaaacattaaaaagagtcacccagatgtcatgttgtatcaatatatggatgatttgttgattgggtctaatagggatgatcataagcaaatagtgcaggaaatcagggataagttaggatcatatggtttcaagactccagatgaaaaggtccaggaagagagagtgaaatggatcggttttgagctcacacccaagaaatggcgttttcagcccaggcaactaaagataaaaaacccactcacagtaaatgaattacagcaattagtaggtaattgtgtttgggtacagccagaagtaaaaatccctctataccccttaaccgatctactgagggataagaccaatctccaagaaaagatacaactaacaccagaagccatcaagtgtgtagaagaattcaatctaaaactaaaagatccagaatggaaagatagaataagagaaggagcagaattagtcataaaaatacagatggttcctcggggcatagtatttgatctgttgcaagatggaaatcccatatggggaggagtaaaaggactaaattatgatcattcaaacaaaataaaaaagatacttagaactatgaatgagctgaacagaacagtggtaattatgacaggaagagaagctagtttcctgcttcctgggtcttctgaagattgggaagcggcactccagaaggaagaaagtctaacacaaatattcccagtaaagttttataggcactcctgcagatggacctccatatgtgggccagtaagagaaaatctaaccacctactatactgacggagggaagaaagggaaaacagctgcagcagtatattggtgtgaaggaaggactaagtcaaaggtatttccaggaaccaatcaacaggcggaattgaaggccatatgcatggctctcttggatggaccaccaaaaatgaatatcataacagatagtagatacgcctatgagggaatgagagaagaaccagaaacgtgggccagggaaggaatctggctggagattgccaagatattgccctttaagcagtacgtgggggtcgggtgggtgcctgcacataaagggataggaggaaatacagaggcagatgaaggagttaagaaagccttagaacagatggccccgtgtagccctcctgaggccattctattaaaaccaggagaaaaacaaaatctggagacagggatctacatgcaggggcttagaccacaaagcttcctcccaagagcagacttaccagtagccatcacaggaaccatggtagattcagagctacagctacagctacttaacataggaactgagcatataagaatccaaaaagatgaggtcttcatgacctgtttcctagaaaatatcccctcagccactgaagatcatgagagatggcatacctcaccagacattttggttaggcagttccatctccctaagagaatagctaaagagatagtagccagatgccaagaatgtaaaaggacaaccactagcccagtcagaggaacaaaccccagaggtcgattcttatggcagatggacaatactcactggaataaaacaattatttgggtagcagtagagacaaattcaggattagtggaagctcaggtgatccctgaagaaacagcactacaagtagctctctgcattttacagctaatccagagatatacagttcttcacttacatagtgacaacgggccgtgctttactgcacacaggatagaaaatctatgtaagtatctggggatcacaaaaactacgggaataccctacaacccacaatcccagggagttgtagaaagagcccacagagatctaaaagacagattggcagcttatcagggagattgtgaaaccgtagaagcagcccttagcctcgcattagtttctttaaataaaaaaagagggggaatagggggccatacaccatatgaaatatacctagaatcagaacataccaaataccaagaccaactagaacaacaattttcaaaacaaaaaattgaaaagtggtgttacgtaaggaacagaagaaaggaatggaaaggaccctacaaagtgttgtgggacggagacggggcagcagtaatagaggaagagggaaaaacagccttatatccacaccgtcatatgcgcttcatcccccccccagattcagatatccaagatgggagttcgtga

SEQ ID NO: 51 - protein, 1035 aa, Bovine immunodeficiency virus
  MISC_FEATURE: BIV Pol Amino Acid Sequence
REVLLCPLWAEEPTTEQFSPEQHEFCDPICTPSYIRLDKQPFIKVFIGGRWVKGLVDTGADEVVLKNIHWDRIKGYPGTPIKQIGVNGVNVAKRKTHVEWRFKDKTGIIDVLFSDTPVNLFGRSLLRSIVTCFTLLVHTEKIEPLPVKVRGPGPKVPQWPLTKEKYQALKEIVKDLLAEGKISEAAWDNPYNTPVFVIKKKGTGRWRMLMDFRELNKITVKGQEFSTGLPYPPGIKECEHLTAIDIKDAYFTIPLHEDFRPFTAFSVVPVNREGPIERFQWNVLPQGWVCSPAIYQTTTQKIIENIKKSHPDVMLYQYMDDLLIGSNRDDHKQIVQEIRDKLGSYGFKTPDEKVQEERVKWIGFELTPKKWRFQPRQLKIKNPLTVNELQQLVGNCVWVQPEVKIPLYPLTDLLRDKTNLQEKIQLTPEAIKCVEEFNLKLKDPEWKDRIREGAELVIKIQMVPRGIVFDLLQDGNPIWGGVKGLNYDHSNKIKKILRTMNELNRTVVIMTGREASFLLPGSSEDWEAALQKEESLTQIFPVKFYRHSCRWTSICGPVRENLTTYYTDGGKKGKTAAAVYWCEGRTKSKVFPGTNQQAELKAICMALLDGPPKMNIITDSRYAYEGMREEPETWAREGIWLEIAKILPFKQYVGVGWVPAHKGIGGNTEADEGVKKALEQMAPCSPPEAILLKPGEKQNLETGIYMQGLRPQSFLPRADLPVAITGTMVDSELQLQLLNIGTEHIRIQKDEVFMTCFLENIPSATEDHERWHTSPDILVRQFHLPKRIAKEIVARCQECKRTTTSPVRGTNPRGRFLWQMDNTHWNKTIIWVAVETNSGLVEAQVIPEETALQVALCILQLIQRYTVLHLHSDNGPCFTAHRIENLCKYLGITKTTGIPYNPQSQGVVERAHRDLKDRLAAYQGDCETVEAALSLALVSLNKKRGGIGGHTPYEIYLESEHTKYQDQLEQQFSKQKIEKWCYVRNRRKEWKGPYKVLWDGDGAAVIEEEGKTALYPHRHMRFIPPPDSDIQDGSS

SEQ ID NO: 52 - DNA, 3108 bp, Artificial Sequence
  Recoded BIV Pol DNA Sequence
cgggaagtgctcctctgccccttatgggcagaggagccaaccacagaacaattttcaccagagcaacatgagttctgtgaccccatctgcaccccctcttatattagattagacaaacagccttttataaaggtgttcattggcggccgctgggtgaagggactggtggacacaggcgctgacgaggtggtgctgaagaacatccactgggaccgcatcaaaggctaccctggaacacccatcaagcagatcggcgtgaacggcgtgaacgtggctaagcgcaaaacacatgtggagtggagattcaaagacaagaccggcatcattgacgtcctcttcagcgacacacctgtgaacctgtttggcagaagcctgctcagatccatcgtgacctgctttaccctgctggtgcacaccgagaagatcgagccactgcctgtgaaggtgcgcggccctggacctaaggtgccacaatggcccctgaccaaggagaaataccaggccctgaaggagatcgtgaaggacctgctggccgagggaaagatcagcgaagctgcctgggacaacccttacaacacacccgtgttcgtgatcaagaagaaaggcaccggccgctggcgcatgctgatggacttccgcgagctgaataagatcaccgtgaaaggccaagagttcagcacaggactcccttatccacccggcatcaaggagtgtgagcacctgaccgccatcgacatcaaggacgcctacttcaccatccctctgcacgaggacttcagacccttcacagccttcagcgtggtcccagtgaaccgcgagggccccatcgagcgcttccagtggaacgtcctgcctcaaggctgggtgtgctcccctgccatctaccagaccacaacccagaagatcattgagaacatcaagaagagccatcccgacgtgatgctgtatcagtacatggatgacctcctgattggcagcaatcgcgatgaccacaagcagatcgtgcaggagatcagagacaagctgggcagctatggcttcaagacacccgacgagaaagtgcaggaagagcgcgtgaagtggatcggcttcgagctgacacctaagaaatggagattccagcctaggcaactgaagatcaagaacccactgaccgtgaacgaactccagcagctggtcggcaactgtgtgtgggtgcagcccgaggtgaagatccctctgtacccactgaccgatctgctccgcgacaagaccaacctgcaggaaaagatccagctgacacccgaggccatcaagtgcgtggaagagttcaacctgaagctgaaagatcccgagtggaaggacagaattcgcgaaggagccgagctggtgatcaagatccaaatggtccctcgcggcatcgtgttcgacctgctgcaagacggcaatcctatctggggaggcgtgaaaggactgaactacgaccacagcaacaagatcaagaagatcctgcgcaccatgaacgagctgaaccgcaccgtggtgatcatgaccggacgcgaagctagctttctcctgcctggatccagcgaggattgggaggccgccctgcagaaggaagagagcctgacccaaatctttcccgtgaagttctaccgccatagctgtagatggacaagcatctgtggacccgtccgcgagaacctgaccacctactataccgacggcgggaagaaaggaaagacagctgccgcagtgtactggtgtgaaggaagaactaagagcaaagtgttccctggaaccaatcaacaggctgagctgaaggcaatctgcatggctctgctggacggacctcccaagatgaacatcatcaccgacagccgctacgcttatgagggcatgagagaggaacctgagacctgggctcgcgagggcatctggctggagattgcaaagatcctgccattcaagcaatacgtcggagtgggctgggtccctgctcacaaaggcattggaggcaataccgaggctgacgaaggagtgaagaaagccctggagcaaatggcaccatgttcccctcccgaggctatcctgctcaaacctggcgagaagcaaaacctggagaccggcatctacatgcaaggcctgagacctcagagcttcctgccccgcgctgacctccctgtcgcaatcactggcaccatggtggactccgagctgcagctccaactgctgaacatcggcaccgagcacattcgcatccagaaggacgaggtgttcatgacatgcttcctggagaacatccctagcgccaccgaagaccacgagagatggcacacatccccagacatcctggtccgccagttccacctgcccaagcgcatcgccaaggagatcgtcgcccgctgccaggagtgcaagagaaccacaacctccccagtgcgcggcaccaaccctagaggacgcttcctgtggcagatggacaacacacactggaacaaaaccatcatttgggtcgcagtggagactaacagcggactggtggaggctcaggtgattcccgaagagaccgcactgcaagtggccctgtgtatcctccagctgatccaacgctacaccgtcctgcacctgcacagcgacaacggaccctgcttcacagctcaccgcatcgagaacctgtgcaagtacctgggcatcaccaagacaaccggcattccctacaatcctcagagccaaggagtcgtggaaagagcccatcgcgacctgaaggacagactggctgcctatcaaggcgactgcgagaccgtggaagctgcactgagcctcgccctggtcagcctgaacaagaagagaggaggcatcggcggacacacaccctacgagatctatctggagagcgagcacaccaagtatcaggaccaactggagcagcaattcagcaagcagaagatcgagaaatggtgctacgtccgcaacagacgcaaggagtggaagggcccttacaaggtgctgtgggatggcgacggagctgcagtgatcgaggaagagggcaagaccgctctgtatccccaccggcacatgcgcttcatcccacctcccgacagcgatatccaggacggctccagctga

SEQ ID NO: 53 - DNA, 3111 bp, Artificial Sequence
  Wild Type BIV Pol Sequence with ATG
atgcgggaagtgctcctctgccccttatgggcagaggagccaaccacagaacaattttcaccagagcaacatgagttctgtgaccccatctgcaccccctcttatattagattagacaaacagccttttataaaggtgttcatagggggaagatgggtaaaagggttagtagacactggagcagatgaggtagtgcttaagaacatacattgggataggataaaagggtatccagggacaccaattaaacaaattggggtaaatggagtaaatgtggccaaaaggaagacccacgtagagtggagatttaaggataagactgggataattgatgtcttgttctcagatactcctgtaaacctttttgggagatctcttctacgtagcatagtgacttgcttcaccctacttgttcacacagaaaaaatcgaacccctacccgtcaaggtaaggggaccagggcctaaggtaccccagtggcccttgacaaaagaaaagtatcaggctcttaaggaaattgtgaaagatcttttagcagaaggaaaaatttccgaagctgcttgggataacccatataataccccagtttttgttataaagaaaaagggaacgggaagatggaggatgctaatggattttagggaattaaataagataacagttaaaggacaagaattctctacaggcttaccttaccctccaggaattaaggaatgtgaacacttaactgcaatagatataaaagatgcctactttactatccctttacatgaggactttagaccctttacagccttctctgtagtccctgtaaatcgagaaggacctatagagaggttccagtggaatgttctaccacaaggatgggtatgtagccctgccatttatcagactaccacccagaagattatagaaaacattaaaaagagtcacccagatgtcatgttgtatcaatatatggatgatttgttgattgggtctaatagggatgatcataagcaaatagtgcaggaaatcagggataagttaggatcatatggtttcaagactccagatgaaaaggtccaggaagagagagtgaaatggatcggttttgagctcacacccaagaaatggcgttttcagcccaggcaactaaagataaaaaacccactcacagtaaatgaattacagcaattagtaggtaattgtgtttgggtacagccagaagtaaaaatccctctataccccttaaccgatctactgagggataagaccaatctccaagaaaagatacaactaacaccagaagccatcaagtgtgtagaagaattcaatctaaaactaaaagatccagaatggaaagatagaataagagaaggagcagaattagtcataaaaatacagatggttcctcggggcatagtatttgatctgttgcaagatggaaatcccatatggggaggagtaaaaggactaaattatgatcattcaaacaaaataaaaaagatacttagaactatgaatgagctgaacagaacagtggtaattatgacaggaagagaagctagtttcctgcttcctgggtcttctgaagattgggaagcggcactccagaaggaagaaagtctaacacaaatattcccagtaaagttttataggcactcctgcagatggacctccatatgtgggccagtaagagaaaatctaaccacctactatactgacggagggaagaaagggaaaacagctgcagcagtatattggtgtgaaggaaggactaagtcaaaggtatttccaggaaccaatcaacaggcggaattgaaggccatatgcatggctctcttggatggaccaccaaaaatgaatatcataacagatagtagatacgcctatgagggaatgagagaagaaccagaaacgtgggccagggaaggaatctggctggagattgccaagatattgccctttaagcagtacgtgggggtcgggtgggtgcctgcacataaagggataggaggaaatacagaggcagatgaaggagttaagaaagccttagaacagatggccccgtgtagccctcctgaggccattctattaaaaccaggagaaaaacaaaatctggagacagggatctacatgcaggggcttagaccacaaagcttcctcccaagagcagacttaccagtagccatcacaggaaccatggtagattcagagctacagctacagctacttaacataggaactgagcatataagaatccaaaaagatgaggtcttcatgacctgtttcctagaaaatatcccctcagccactgaagatcatgagagatggcatacctcaccagacattttggttaggcagttccatctccctaagagaatagctaaagagatagtagccagatgccaagaatgtaaaaggacaaccactagcccagtcagaggaacaaaccccagaggtcgattcttatggcagatggacaatactcactggaataaaacaattatttgggtagcagtagagacaaattcaggattagtggaagctcaggtgatccctgaagaaacagcactacaagtagctctctgcattttacagctaatccagagatatacagttcttcacttacatagtgacaacgggccgtgctttactgcacacaggatagaaaatctatgtaagtatctggggatcacaaaaactacgggaataccctacaacccacaatcccagggagttgtagaaagagcccacagagatctaaaagacagattggcagcttatcagggagattgtgaaaccgtagaagcagcccttagcctcgcattagtttctttaaataaaaaaagagggggaatagggggccatacaccatatgaaatatacctagaatcagaacataccaaataccaagaccaactagaacaacaattttcaaaacaaaaaattgaaaagtggtgttacgtaaggaacagaagaaaggaatggaaaggaccctacaaagtgttgtgggacggagacggggcagcagtaatagaggaagagggaaaaacagccttatatccacaccgtcatatgcgcttcatcccccccccagattcagatatccaagatgggagttcgtga

SEQ ID NO: 54 - DNA, 3111 bp, Artificial Sequence
  Recoded BIV Pol DNA Sequence With ATG
atgcgggaagtgctcctctgccccttatgggcagaggagccaaccacagaacaattttcaccagagcaacatgagttctgtgaccccatctgcaccccctcttatattagattagacaaacagccttttataaaggtgttcattggcggccgctgggtgaagggactggtggacacaggcgctgacgaggtggtgctgaagaacatccactgggaccgcatcaaaggctaccctggaacacccatcaagcagatcggcgtgaacggcgtgaacgtggctaagcgcaaaacacatgtggagtggagattcaaagacaagaccggcatcattgacgtcctcttcagcgacacacctgtgaacctgtttggcagaagcctgctcagatccatcgtgacctgctttaccctgctggtgcacaccgagaagatcgagccactgcctgtgaaggtgcgcggccctggacctaaggtgccacaatggcccctgaccaaggagaaataccaggccctgaaggagatcgtgaaggacctgctggccgagggaaagatcagcgaagctgcctgggacaacccttacaacacacccgtgttcgtgatcaagaagaaaggcaccggccgctggcgcatgctgatggacttccgcgagctgaataagatcaccgtgaaaggccaagagttcagcacaggactcccttatccacccggcatcaaggagtgtgagcacctgaccgccatcgacatcaaggacgcctacttcaccatccctctgcacgaggacttcagacccttcacagccttcagcgtggtcccagtgaaccgcgagggccccatcgagcgcttccagtggaacgtcctgcctcaaggctgggtgtgctcccctgccatctaccagaccacaacccagaagatcattgagaacatcaagaagagccatcccgacgtgatgctgtatcagtacatggatgacctcctgattggcagcaatcgcgatgaccacaagcagatcgtgcaggagatcagagacaagctgggcagctatggcttcaagacacccgacgagaaagtgcaggaagagcgcgtgaagtggatcggcttcgagctgacacctaagaaatggagattccagcctaggcaactgaagatcaagaacccactgaccgtgaacgaactccagcagctggtcggcaactgtgtgtgggtgcagcccgaggtgaagatccctctgtacccactgaccgatctgctccgcgacaagaccaacctgcaggaaaagatccagctgacacccgaggccatcaagtgcgtggaagagttcaacctgaagctgaaagatcccgagtggaaggacagaattcgcgaaggagccgagctggtgatcaagatccaaatggtccctcgcggcatcgtgttcgacctgctgcaagacggcaatcctatctggggaggcgtgaaaggactgaactacgaccacagcaacaagatcaagaagatcctgcgcaccatgaacgagctgaaccgcaccgtggtgatcatgaccggacgcgaagctagctttctcctgcctggatccagcgaggattgggaggccgccctgcagaaggaagagagcctgacccaaatctttcccgtgaagttctaccgccatagctgtagatggacaagcatctgtggacccgtccgcgagaacctgaccacctactataccgacggcgggaagaaaggaaagacagctgccgcagtgtactggtgtgaaggaagaactaagagcaaagtgttccctggaaccaatcaacaggctgagctgaaggcaatctgcatggctctgctggacggacctcccaagatgaacatcatcaccgacagccgctacgcttatgagggcatgagagaggaacctgagacctgggctcgcgagggcatctggctggagattgcaaagatcctgccattcaagcaatacgtcggagtgggctgggtccctgctcacaaaggcattggaggcaataccgaggctgacgaaggagtgaagaaagccctggagcaaatggcaccatgttcccctcccgaggctatcctgctcaaacctggcgagaagcaaaacctggagaccggcatctacatgcaaggcctgagacctcagagcttcctgccccgcgctgacctccctgtcgcaatcactggcaccatggtggactccgagctgcagctccaactgctgaacatcggcaccgagcacattcgcatccagaaggacgaggtgttcatgacatgcttcctggagaacatccctagcgccaccgaagaccacgagagatggcacacatccccagacatcctggtccgccagttccacctgcccaagcgcatcgccaaggagatcgtcgcccgctgccaggagtgcaagagaaccacaacctccccagtgcgcggcaccaaccctagaggacgcttcctgtggcagatggacaacacacactggaacaaaaccatcatttgggtcgcagtggagactaacagcggactggtggaggctcaggtgattcccgaagagaccgcactgcaagtggccctgtgtatcctccagctgatccaacgctacaccgtcctgcacctgcacagcgacaacggaccctgcttcacagctcaccgcatcgagaacctgtgcaagtacctgggcatcaccaagacaaccggcattccctacaatcctcagagccaaggagtcgtggaaagagcccatcgcgacctgaaggacagactggctgcctatcaaggcgactgcgagaccgtggaagctgcactgagcctcgccctggtcagcctgaacaagaagagaggaggcatcggcggacacacaccctacgagatctatctggagagcgagcacaccaagtatcaggaccaactggagcagcaattcagcaagcagaagatcgagaaatggtgctacgtccgcaacagacgcaaggagtggaagggcccttacaaggtgctgtgggatggcgacggagctgcagtgatcgaggaagagggcaagaccgctctgtatccccaccggcacatgcgcttcatcccacctcccgacagcgatatccaggacggctccagctga

SEQ ID NO: 55 - protein, 29 aa, Human immunodeficiency virus
  MISC_FEATURE: Partial Amino Acid Sequence HIV protease
PQVTLWQRPLVTIKIGGQLKEALLDTGAD

SEQ ID NO: 56 - protein, 29 aa, Bovine immunodeficiency virus
  MISC_FEATURE: Partial Amino Acid Sequence of BIV Protease
SYIRLDKQPFIKVFIGGRWVKGLVDTGAD

SEQ ID NO: 57 - protein, 29 aa, Artificial Sequence
  Partial amino acid sequence of mutated HIV HXB2 protease
PQVTLWQRPLVTIKIGGQLKEALLDSGAD

SEQ ID NO: 58 - protein, 29 aa, Artificial Sequence
  A point mutated 127 isolate
SYIRLDKQPFIKVFIGGRWVKGLVDSGAD

SEQ ID NO: 59 - DNA, 4427 bp, Artificial Sequence
  Recoded gag/pol with protease mutation
atgaagcggagagagctggagaagaaactgaggaaagtgcgcgtgacacctcaacaggacaagtactataccatcggcaacctgcagtgggccatccgcatgatcaacctgatgggcatcaagtgcgtgtgcgacgaggaatgcagcgccgctgaggtcgccctgatcatcacccagtttagcgccctcgacctggagaactcccctatccgcggcaaggaagaggtggccatcaagaataccctgaaggtgttttggagcctgctggccggatacaagcctgagagcaccgagaccgccctgggatactgggaagccttcacctacagagagagggaagctagagccgacaaggagggagagatcaagagcatctaccctagcctgacccagaacacccagaacaagaaacagaccagcaatcagacaaacacccagagcctgcccgctatcaccacacaggatggcacccctcgcttcgaccccgacctgatgaagcagctgaagatctggtccgatgccacagagcgcaatggagtggacctgcatgccgtgaacatcctgggagtgatcacagccaacctggtgcaagaagagatcaagctcctgctgaatagcacacccaagtggcgcctggacgtgcagctgatcgagagcaaagtgagagagaaggagaacgcccaccgcacctggaagcagcatcaccctgaggctcccaagacagacgagatcattggaaagggactgagctccgccgagcaggctaccctgatcagcgtggagtgcagagagaccttccgccagtgggtgctgcaggctgccatggaggtcgcccaggctaagcacgccacacccggacctatcaacatccatcaaggccctaaggaaccctacaccgacttcatcaaccgcctggtggctgccctggaaggaatggccgctcccgagaccacaaaggagtacctcctgcagcacctgagcatcgaccacgccaacgaggactgtcagtccatcctgcgccctctgggacccaacacacctatggagaagaaactggaggcctgtcgcgtggtgggaagccagaagagcaagatgcagttcctggtggccgctatgaaggaaatggggatccagtctcctattccagccgtgctgcctcacacacccgaagcctacgcctcccaaacctcagggcccgaggatggtaggagatgttacggatgtgggaagacaggacatttgaagaggaattgtaaacagcaaaaatgctaccattgtggcaaacctggccaccaagcaagaaactgcaggtcaaaaaacgggaagtgctcctctgccccttatgggcagaggagccaaccacagaacaattttcaccagagcaacatgagttctgtgaccccatctgcaccccctcttatattagattagacaaacagccttttataaaggtgttcattggcggccgctgggtgaagggactggtggactcaggcgctgacgaggtggtgctgaagaacatccactgggaccgcatcaaaggctaccctggaacacccatcaagcagatcggcgtgaacggcgtgaacgtggctaagcgcaaaacacatgtggagtggagattcaaagacaagaccggcatcattgacgtcctcttcagcgacacacctgtgaacctgtttggcagaagcctgctcagatccatcgtgacctgctttaccctgctggtgcacaccgagaagatcgagccactgcctgtgaaggtgcgcggccctggacctaaggtgccacaatggcccctgaccaaggagaaataccaggccctgaaggagatcgtgaaggacctgctggccgagggaaagatcagcgaagctgcctgggacaacccttacaacacacccgtgttcgtgatcaagaagaaaggcaccggccgctggcgcatgctgatggacttccgcgagctgaataagatcaccgtgaaaggccaagagttcagcacaggactcccttatccacccggcatcaaggagtgtgagcacctgaccgccatcgacatcaaggacgcctacttcaccatccctctgcacgaggacttcagacccttcacagccttcagcgtggtcccagtgaaccgcgagggccccatcgagcgcttccagtggaacgtcctgcctcaaggctgggtgtgctcccctgccatctaccagaccacaacccagaagatcattgagaacatcaagaagagccatcccgacgtgatgctgtatcagtacatggatgacctcctgattggcagcaatcgcgatgaccacaagcagatcgtgcaggagatcagagacaagctgggcagctatggcttcaagacacccgacgagaaagtgcaggaagagcgcgtgaagtggatcggcttcgagctgacacctaagaaatggagattccagcctaggcaactgaagatcaagaacccactgaccgtgaacgaactccagcagctggtcggcaactgtgtgtgggtgcagcccgaggtgaagatccctctgtacccactgaccgatctgctccgcgacaagaccaacctgcaggaaaagatccagctgacacccgaggccatcaagtgcgtggaagagttcaacctgaagctgaaagatcccgagtggaaggacagaattcgcgaaggagccgagctggtgatcaagatccaaatggtccctcgcggcatcgtgttcgacctgctgcaagacggcaatcctatctggggaggcgtgaaaggactgaactacgaccacagcaacaagatcaagaagatcctgcgcaccatgaacgagctgaaccgcaccgtggtgatcatgaccggacgcgaagctagctttctcctgcctggatccagcgaggattgggaggccgccctgcagaaggaagagagcctgacccaaatctttcccgtgaagttctaccgccatagctgtagatggacaagcatctgtggacccgtccgcgagaacctgaccacctactataccgacggcgggaagaaaggaaagacagctgccgcagtgtactggtgtgaaggaagaactaagagcaaagtgttccctggaaccaatcaacaggctgagctgaaggcaatctgcatggctctgctggacggacctcccaagatgaacatcatcaccgacagccgctacgcttatgagggcatgagagaggaacctgagacctgggctcgcgagggcatctggctggagattgcaaagatcctgccattcaagcaatacgtcggagtgggctgggtccctgctcacaaaggcattggaggcaataccgaggctgacgaaggagtgaagaaagccctggagcaaatggcaccatgttcccctcccgaggctatcctgctcaaacctggcgagaagcaaaacctggagaccggcatctacatgcaaggcctgagacctcagagcttcctgccccgcgctgacctccctgtcgcaatcactggcaccatggtggactccgagctgcagctccaactgctgaacatcggcaccgagcacattcgcatccagaaggacgaggtgttcatgacatgcttcctggagaacatccctagcgccaccgaagaccacgagagatggcacacatccccagacatcctggtccgccagttccacctgcccaagcgcatcgccaaggagatcgtcgcccgctgccaggagtgcaagagaaccacaacctccccagtgcgcggcaccaaccctagaggacgcttcctgtggcagatggacaacacacactggaacaaaaccatcatttgggtcgcagtggagactaacagcggactggtggaggctcaggtgattcccgaagagaccgcactgcaagtggccctgtgtatcctccagctgatccaacgctacaccgtcctgcacctgcacagcgacaacggaccctgcttcacagctcaccgcatcgagaacctgtgcaagtacctgggcatcaccaagacaaccggcattccctacaatcctcagagccaaggagtcgtggaaagagcccatcgcgacctgaaggacagactggctgcctatcaaggcgactgcgagaccgtggaagctgcactgagcctcgccctggtcagcctgaacaagaagagaggaggcatcggcggacacacaccctacgagatctatctggagagcgagcacaccaagtatcaggaccaactggagcagcaattcagcaagcagaagatcgagaaatggtgctacgtccgcaacagacgcaaggagtggaagggcccttacaaggtgctgtgggatggcgacggagctgcagtgatcgaggaagagggcaagaccgctctgtatccccaccggcacatgcgcttcatcccacctcccgacagcgatatccaggacggctccagctga

SEQ ID NO: 60 - DNA, 468 bp, mouse
  misc_feature: Mouse RdCVF1 cDNA
atcggatccctctctgggtccccagctccttgcatactgctaccatggcatctctcttctctggacgcatcttgatcaggaacaacagcgaccaggatgaagtggagacagaggcagagctgagccgtaggttagagaatcgtctggtgttgctgttcttcggcgccggcgcctgtccccagtgccaggcctttgccccagtcctcaaagacttcttcgtgcggctcactgacgagttctacgtgctgcgggcagcacagctggccctggtctatgtgtcccaggaccctacagaggagcaacaggacctcttcctcagggacatgcctgaaaaatggctcttcctgccgttccatgatgaactgaggaggtgaggccccagggaagaccagggagggcttcctggagaaggcatttccctggaggtttactgtcctggtactacttgtgcataaagaggtattcctc

SEQ ID NO: 61 - protein, 109 aa, mouse
  MISC_FEATURE: Amino acid sequence of translated mouse RdCVF1 cDNA
MASLFSGRILIRNNSDQDEVETEAELSRRLENRLVLLFFGAGACPQCQAFAPVLKDFFVRLTDEFYVLRAAQLALVYVSQDPTEEQQDLFLRDMPEKWLFLPFHDELRR

SEQ ID NO: 62 - DNA, 353 bp, human
  misc_feature: Human RdCVF1 cDNA
cccagcacccaacccaggttaccatggcctccctgttctctggccgcatcctgatccgcaacaatagcgaccaggacgagctggatacggaggctgaggtcagtcgcaggctggagaaccggctggtgctgctgttctttggtgctggggcttgtccacagtgccaggccttcgtgcccatcctcaaggacttcttcgtgcggctcacagatgagttctatgtactgcgggcggctcagctggccctggtgtacgtgtcccaggactccacggaggagcagcaggacctgttcctcaaggacatgccaaagaaatggcttttcctgccctttgaggatgatctgaggaggtga

SEQ ID NO: 63 - protein, 109 aa, human
  MISC_FEATURE: Amino acid sequence of translated human RdCVF1 cDNA
MASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 64 - DNA, 600 bp, mouse
  misc_feature: Mouse RdCVF2 cDNA
ataaaatagagggtgggagaggttgatggcgtggctctgctttttggtgcggggcacccagctgtcatcgctgctgtcgcagcttctggagtggccactgtgctctctcctcccttcggctcaaggtgagctgttccagcagaaggcggggctgagaggcgcctagtgctgcgggaggctcagtgtcatcttccagctaacaggtggccgtgcagcccagggctcgtctctccactgtgtcctcttcacgccgagctcgtggcgatggtggacgtgctgggcgggcggcgcctggtgacccgggagggcacggtggtggaggccgaggtggcgctgcagaacaaggtggtagctttgtactttgcggcgggccggtgctcgcccagccgcgacttcacgccgctgctctgcgacttctacacggagctggtgagcgaggcgcggcggcccgctcccttcgaggtggttttcgtgtcggcagacggcagtgcggaggagatgttggacttcatgcgcgagctgcacggctcctggctggcattgcccttccacgacccctaccggcagtgagtggggacccaggggtcatggggctggcgc

SEQ ID NO: 65 - protein, 101 aa, mouse
  MISC_FEATURE: Amino acid sequence of translated mouse RdCVF2 cDNA
MVDVLGGRRLVTREGTVVEAEVALQNKVVALYFAAGRCSPSRDFTPLLCDFYTELVSEARRPAPFEVVFVSADGSAEEMLDFMRELHGSWLALPFHDPYRQ

SEQ ID NO: 66 - DNA, 1472 bp, mouse
  misc_feature: Human RdCVF2 cDNA
gtgtgggcggggcgcagttgggggagggtgcagagacctgagggcttgaggttgcctggctggccccgctcccagaggcgggtgccgcgctgtcgcccaggtatctggggtctctggtgtctgagtgtctcattgtcggcgcgaacacaattgctccagccacaggcgaggcctggccaaggtgtgggcgcatctagggcaggtcttgagaggtccagcgcccggtggtgcggacagaggcggggcaccgcggcgctcgccgccgcctccccgcaggtgatcatcctcctgcaggtgtcctcgggtctcaggtggctgcgtgtctgcgccatggttgacattctgggcgagcggcacctggtgacctgtaagggcgcgacggtggaggccgaggcggcgctgcagaacaaggtggtggcactgtacttcgcggcggcccggtgcgcgccgagccgcgacttcacgccgctgctctgcgacttctatacggcgctggtggccgaggcgcggcggcccgcgcccttcgaagtggtcttcgtgtcagccgacggcagctgccaggagatgctggacttcatgcgcgagctgcatggcgcctggctggcgctgcccttccacgacccctaccggcaacggagtctcgctctgttgcccaggctggagtgcagtggcgtgatcttagctcactgcaacctttgcctcctgggttcaagtgattctctagccttagcctcctgagcatctgggactacagccattgctgtgaattacgtgagggaaagatattgaagaggagttggacactccgagagtgcagctgttctccccccgcaccatccgtgtcctgcattctgcgagtctgtgctcattaacaatgtgctgtgaccatgtgactcagcaatcctgctgctgggtatatacccgaaagaaaggaaaaggaagccagtatattgaagaggtatctgcacccccatgtttattgcagcactgttcacaacagccaagatttggaagcaacctaagtgtccatcaacagatgaatggataaagaaaacgtggtacatatacacaatggagtactcttcagccattaaaaaaatgagattctgtcatttgcaataatatagatggaaaaggaggcccttatgtgaagtgaaataagccaggcacagaaagacaaacatcacatgttctcacttatttgtgggatctaatgatcaaaacaattgaactcttggacatagagagtagaaggttggttaccagaagctggaaaggaaagtggggttgggaggaaggtgggaatggttaataggtacaaaaaaatacaaagaataaataagacctaatatttgatagcacaacagtgtgactactgtcaataatcatttaattgtacatttaaaaataactataattgcattgtttgtaacacaaaagataaatgcttgaggagaaaaaaaaaaaaaaaa

SEQ ID NO: 67 - protein, 135 aa, human
  MISC_FEATURE: Amino acid sequence of translated human RdCVF2 cDNA
MVDILGERHLVTCKGATVEAEAALQNKVVALYFAAARCAPSRDFTPLLCDFYTALVAEARRPAPFEVVFVSADGSCQEMLDFMRELHGAWLALPFHDPYRQRSLALLPRLECSGVILAHCNLCLLGSSDSLALAS

SEQ ID NO: 68 - DNA, 1539 bp, Thogoto virus
  CDS (1)..(1539)
atgttccttcagactgcactgcttctgctatccttaggggtagcagaacctgactgcaatacaaaaacagccacaggcccatatatattggacagatataaacccaagccagtcactgtatccaagaagttgtattcggccaccagatacacaacctctgcacaaaatgagctacttaccgctggttatcgcacagcctgggtggcttactgctataacggtggcctcgttgattctaatactggttgtaatgctaggctactgcattacccgcccagcagagatgagttactgctttggggatcatctcaccagtgttcatacggggacatctgccatgattgctgggggagcgattcctatgcatgcctgggacagctggaccctgccaaacattgggcgccaaggaaggagctcgtcagaagagatgctaactggaaattcgcataccatatgtgcaacatcgactggagatgcggagtcaccacctcccccgtattcttcaatttgcagtgggtaaagaatgaagtaaaggtcagcactcttctgcctaatggaagcactgtggaacactctgctggggagcctctgttctggactgagaaggacttctcatatctggtcaaagacaatttcgaaatacagagggaagaagtaaaaatcagctgcttcgtagatccagactattgggtaggagaaaggaagaccaagaaagcgttttgccaagacgggaccaacttcttcgaagtgacttcacaccaattttgtcatcaatatgcatgttacaacttctctaaggacgaactgctggaagctgtctacaaagaaagagctcacgagaaaagcaaggacctgccctttggtaataaaagctggactgtggtgacagcctccatagatgacctccacgcactcagtgcagcacaggcatttgagctggaagggctcagagcatcttttgctgaactggattcacgatttaggcagctgtcagaaattttggacacagtgatatccagtatcgccaagatcgacgagagacttatcggcagactgatcaaagcacccgtttctagcagattcatctcagaggacaagtttctgctacatcagtgcgtggacagtgtggccaacaacaccaactgcgtgggagacagtgcatatgtggacggcaggtggacccatgttggggacaaccatccttgcacaaccgtggtagatgaaccaataggcattgacatctataacttcagtgccctctggtacccttctgccgccgaagtcgattttaggggaactgtccagtcagaagatggttggtcttttgtggtcaaatctaaagacgcgctcatccagaccatgatgtacaccaaaaacggcggtaaagggacttctctcacggacctcttggactacccttctggttggcttaaggggcagctggggggcttgctatatggcaatatcggtgtgtacttgttaattgctttcgcttttgtgctattgatcagactaattaagagtgctggattatgctaa

SEQ ID NO: 69 - protein, 512 aa, Thogoto virus
MFLQTALLLLSLGVAEPDCNTKTATGPYILDRYKPKPVTVSKKLYSATRYTTSAQNELLTAGYRTAWVAYCYNGGLVDSNTGCNARLLHYPPSRDELLLWGSSHQCSYGDICHDCWGSDSYACLGQLDPAKHWAPRKELVRRDANWKFAYHMCNIDWRCGVTTSPVFFNLQWVKNEVKVSTLLPNGSTVEHSAGEPLFWTEKDFSYLVKDNFEIQREEVKISCFVDPDYWVGERKTKKAFCQDGTNFFEVTSHQFCHQYACYNFSKDELLEAVYKERAHEKSKDLPFGNKSWTVVTASIDDLHALSAAQAFELEGLRASFAELDSRFRQLSEILDTVISSIAKIDERLIGRLIKAPVSSRFISEDKFLLHQCVDSVANNTNCVGDSAYVDGRWTHVGDNHPCTTVVDEPIGIDIYNFSALWYPSAAEVDFRGTVQSEDGWSFVVKSKDALIQTMMYTKNGGKGTSLTDLLDYPSGWLKGQLGGLLYGNIGVYLLIAFAFVLLIRLIKSAGLC

SEQ ID NO: 70 - DNA, 1567 bp, Thogoto virus
  CDS (19)..(1557): Recoded thogoto virus envelope
ccgctcgagcgggccaccatgttcctgcagacagctctcctgctcctgtccctgggagtggccgaacctgactgcaacaccaagaccgccacaggcccctacattctggaccggtacaagcccaagcccgtgaccgtgagcaagaagctgtactccgccaccagatacaccaccagcgcccagaacgagctgctgacagctggctaccggaccgcttgggtggcctactgctacaacggcggactggtggacagcaacacaggatgcaacgccagactgctgcactatcctccttcccgggacgaactgctcctgtggggctccagccatcagtgtagctacggcgacatctgtcacgactgctggggctccgatagctacgcctgcctgggccagctggatcctgccaagcactgggctcctcggaaggaactggtgagacgggacgccaactggaagttcgcctaccacatgtgcaacatcgactggcggtgcggcgtgaccacaagccctgtcttcttcaacctgcagtgggtcaagaacgaagtgaaggtgagcaccctgctgcccaatggaagcaccgtggagcacagcgctggcgagcccctgttctggaccgagaaggacttcagctacctggtgaaagacaacttcgagatccagcgggaggaggtgaagatcagctgcttcgtggatcctgactactgggtgggcgagcggaagaccaagaaagccttctgtcaggacggcaccaacttcttcgaagtgaccagccaccagttctgccaccagtacgcctgctacaacttctccaaggacgagctgctggaagctgtgtacaaggagcgggcccacgagaagagcaaggacctgcccttcggcaacaagtcctggaccgtggtgaccgcctccatcgacgacctgcatgctctgagcgccgctcaggccttcgaactggagggactgcgggctagctttgctgaactggactccagattccggcagctgagcgagatcctggacaccgtgatcagcagcatcgccaagatcgacgagagactgatcggcagactcatcaaagctcctgtgagcagccggttcatcagcgaggacaagttcctgctgcaccagtgcgtggacagcgtggccaacaacaccaactgtgtgggagacagcgcctacgtggacggccggtggacacatgtgggcgacaatcatccctgcaccacagtggtggacgagcccatcggcatcgatatctacaacttctccgctctgtggtatccttccgccgctgaagtggacttccggggcacagtgcagtccgaggacggctggagcttcgtggtgaagagcaaggacgccctgatccagaccatgatgtacaccaagaacggaggcaagggcacaagcctgaccgacctgctggactaccctagcggatggctgaagggacaactgggcggactgctgtacggcaacatcggagtgtacctgctgatcgccttcgccttcgtgctgctgatcagactgatcaagagcgccggcctgtgctgagctctagagc

SEQ ID NO: 71 - protein, 512 aa, Thogoto virus; identical to SEQ ID NO: 69